(19) United States
(12) Patent Application Publication
(10) Pub. No.: US 2018/0140703 A1
(43) Pub. Date: May 24, 2018

(54) TUMOR RADIOSENSITIZATION WITH ANTIBODY CONJUGATES

(71) Applicant: The Regents of the University California, Oakland, CA (US)

(72) Inventors: Sunil J. Advani, Encinitas, CA (US); Stephen Adams, Poway, CA (US); Roger Y. Tsien, Eugene, OR (US)

(21) Appl. No.: 15/817,017

(22) Filed: Nov. 17, 2017

Related U.S. Application Data
(60) Provisional application No. 62/424,317, filed on Nov. 18, 2016.

(51) Int. Cl. A61K 41/00; C07K 16/28; C07K 16/32; A61K 47/68

(52) U.S. Cl. CPC A61K 41/0038; A61K 47/6803; C07K 16/32; C07K 16/2863

(57) ABSTRACT

Disclosed herein, the invention pertains to methods and compositions that find use in radiosensitization of tumors and tumor samples based on the ability of a tumor sample to cleave a MTS molecule of the present invention. The MTS molecules of the present invention have a formula as disclosed herein and wherein A is a peptide with a sequence comprising 5 to 9 consecutive acidic amino acids, wherein the amino acids are selected from: aspartates and glutamates; B is a peptide with a sequence comprising 5 to 20 consecutive basic amino acids; X and Y are linkers; P is an optional pre-targeting moiety; M is an optional macromolecular carrier; and T is a radiosensitization agent for delivery to a target, including for example a therapeutic compound, wherein T is not an auristatin, including MMAE, or a derivative thereof.

### CROSS-REFERENCE TO RELATED APPLICATION

The present application claims the benefit of U.S. Provisional Application Ser. No. 62/424,317, filed Nov. 18, 2016, the content of which is hereby expressly incorporated herein by reference in its entirety for all purposes.

### STATEMENT OF FEDERALLY-SPONSORED RESEARCH

This invention was made with government support under CA205765, awarded by National Cancer Institute (NCI). The government has certain rights in this invention.

### BACKGROUND OF THE INVENTION

### Introduction

Cell membranes delimit the outer boundaries of cells, and regulate transport into and out of the cell interior. Made primarily of lipids and proteins, cell membranes provide a hydrophilic surface enclosing a hydrophobic interior across which materials must pass before entering a cell. Although many small, lipophilic compounds are able to cross cell membranes passively, most compounds, particles and materials must rely on active mechanisms in order to gain entry into a living cell.

### Transmembrane Transport

Regulation of transport into and out of a cell is vital for its continued viability. For example, cell membranes contain ion channels, pumps, and exchangers capable of facilitating the transmembrane passage of many important substances. However, transmembrane transport is selective: in addition to facilitating the entry of desired substances into a cell, and facilitating the exit of others, a major role of a cell membrane is to prevent uncontrolled entry of substances into the cell interior. This barrier function of the cell membrane makes difficult the delivery of markers, drugs, nucleic acids, and other exogenous material into cells.

Over the last decade, peptide sequences that can readily enter a cell have been identified. For example, the Tat protein of the human immunodeficiency virus 1 (HIV-1) is able to enter cells from the extracellular environment (e.g., Fawell et al. P.N.A.S. 91:664-668 (1994)). Such uptake is reviewed in, for example, Richard et al., J. Biol. Chem. 278(1):585-590 (2003).

Such molecules that are readily taken into cells may also be used to carry other molecules into cells along with them. Molecules that are capable of facilitating transport of substances into cells have been termed “membrane translocation signals” (MTS) as described in Tung et al., Advanced Drug Delivery Reviews 55:281-294 (2003). The most important MTS are rich in amino acids such as arginine with positively charged side chains. Molecules transported into cell by such cationic peptides may be termed “cargo” and may be reversibly or irreversibly linked to the cationic peptides. An example of a reversible linkage is found in Zhang et al., P.N.A.S. 95:9184-9189 (1994)).

MTS molecules are discussed in, for example, Wender et al., P.N.A.S. 97:1300313008 (2000); Hallbrink et al., Biochim. Biophys. Acta 1515:101-109 (2001); Derossi et al., Trends in Cell Biology 8:84-87 (1998); Rothbard et al., 1 Med. Chem. 45:3612-3618 (2002); Rothbard et al., Nature Medicine 6(11):1253-1247 (2000); Wadia et al., Curr. Opinion Biotech. 13:52-56 (2002); Futaki et al.; Bioconj. Chem. 12:1005-1011 (2001); Rothbard et al., U.S. Pat. No. 6,306,993; Frankel et al., U.S. Pat. No. 6,316,003; Rothbard et al., U.S. Pat. No. 6,495,663; Monahan et al., U.S. Pat. No. 6,630,351 and Jiang et al., WO 2005/042034.

### Cancer Surgery

In cancer surgery, positive margins, defined as tumor cells present at the cut edge of the surgical specimen, have been associated with increased local recurrence and a poor prognosis (Hague R., et al., BMC Ear Nose Throat Disord. 16:2 (2006)). As in most solid tumors, salvage surgery (i.e., re-excision of the positive margin) or adjuvant chemotherapy and/or radiation not only cause extra trauma and expense but also often fail to remediate the poor outcome (Hague R., et al., BMC Ear Nose Throat Disord. 16:2 (2006); Singletary S. Am. J. Surg. 184:383-393 (2002); Meric F., et al., Cancer 97:926-933 (2003); Snij der R., et al., Annals of Thoracic Surg. 65 (1998); Nagtegaal I D, Quirke P., J. Clin. On. 26:303-312 (2008); Dotan Z, et al., J. Urol. 178:2308-2312 (2007); and Wieder J. A., J. Urol. 160:299-315 (1998)).

The reason for this observation is likely multifactorial and related in part to the difficulty in identifying the residual cancer during repeat surgery. Therefore, development of more sensitive imaging and diagnostic assays for more accurate detection of positive surgical margins during the primary operation would be one of the most effective means to minimize patient suffering and expense and to improve survival.

As the field of molecularly targeting fluorescent markers for early cancer detection and intraoperative margin evaluation progresses and more enzymatically activatable probes (Jiang T., et al. P.N.A.S. USA. 101:17867-17872 (2004); Aguilera T. A., et al., Integr. Biol. 1:371-381 (2009); Olson E. S., et al., Integr Biol (Camb). 1:382-393 (2009); Olson E. S., et al., PNAS USA. 107:4311-4316 (2010); Nguyen Q. T., PNAS USA. 107:4317-4322 (2010); Blum G., et al., Nat Chem Biol. 3:668-677 (2007); Gounaris E., et al., PLoS One. 3:e2916 (2008); Bremer C., et al., Invest Radiol. 40:321-327 (2005)) are becoming available for clinical use, methods such as those described herein personalized would be useful in a variety of treatment, diagnostic, prognostic applications.

### Radiosensitization

Activatable cell penetrating peptides (ACPPs; also referred to herein as MTS molecules) are peptide based molecules in which a polycationic sequence, typically comprising 8-12 arginines, is connected via an enzyme cleavable linker to a polyanionic sequence, typically comprising a matching number of glutamates. The present application provides disclosure of a new subclass of ACPP (MTS) that accommodates pretargeting agent/ligand and prodrug attached by a linker cleavable after endocytosis. Herein is demonstrated that pre-targeted ACPP produces tumor specific radiosensitization. Such radiosensitizers can include antibody conjugates, inluding those that are currently approved for clinical use.

Conjugating a maytansinoid, such as but not limited to mertansine, to antibodies and/or the ACPPs/MTS molecules of the present invention, tumor specific radiosensitization and tumor regression at much lower dose than what is used for maytansinoid chemotherapy or maytansinoid containing peptide based targeted chemotherapy alone can be achieved.

Overall, there remains a need in the art for additional treatment, specifically methods for radiosensitization or to decrease toxicity and improve efficacy. The present invention meets these needs and provides methods for treatment, diagnosis, prognosis and characterization of tumors which can find use in a variety of personalized medicine applications.

All patents and publications, both supra and infra, are hereby incorporated by reference in their entirety.

### BRIEF DESCRIPTION OF THE DRAWINGS

FIG. 1: Anti-ErbB antibody MMAE conjugates bind in a receptor-dependent manner. (a) CAL-27 (EGFR+) cells exposed to 2 nM of Cy5-labelled C-MMAE for 30 min then incubated in drug-free media. Cells were fixed at indicated times and imaged for Cy5 fluorescence (red). Nuclei stained with DAPI (blue). Scale bar, 10 gm. Representative images of three independent experiments. (b) A panel of EGFR (CAL27, A549, CALU3) and HER2 (CALU3, OE19, BT474) expressing cells from diverse tumour histologies were exposed to 2 nM Cy5-labelled C-MMAE or T-MMAE for 2 h and Cy5 fluorescence (red) imaged. Nuclei stained with DAPI (blue). Scale bar, 10 gm. Representative images of three independent experiments. (c) Cell surface binding of Cy5-labelled C-MMAE or T-MMAE. CAL-27 and OE19 cells incubated on ice with increasing concentrations of C-MMAE or T-MMAE. of Flow cytometry assessment of Cy5 signal. Representative data of two independent experiments. (d) Phase contrast microscopy of CAL-27 cells treated with 2 nM MMAE, cetuximab, or C-MMAE overnight. Representative images of three independent experiments. Scale bar, 50 gm. (e) Cell cycle profile of OE19 cells treated with MMAE, ErbB antibodies (cetuximab or trastuzumab) or ADC (C-MMAE or T-MMAE) overnight, stained with propidium iodide and analysed by flow cytometry. Data representative of two independent experiments.

FIG. 2: Antibody conjugated MMAE selectively radiosensitizes tumour cells. (a) CAL-27 (EGRF+) and OE19 (HER2+) cells were exposed to a dose range of cisplatin, paclitaxel or MMAE for 72-96 h. Cell viability normalized to vehicle treated cells and plotted as mean fractional survival±s.d. of three replicates. Data representative of three independent experiments. (b) CAL-27 and OE19 tumour cells were exposed to dose range of MMAE, ErbB antibody (cetuximab or trastuzumab) or ADC (C-MMAE or T-MMAE) for 72 h. Cell viability plotted as mean fractional survival s.d. of three replicates. Data representative of three independent experiments. (c) Clonogenic cell survival (SF2) of CAL-27 cells treated with MMAE, cetuximab or C-MMAE overnight followed by 0 or 2 Gy. Cell viability normalized to non-irradiated cells for each drug condition and plotted as mean fractional survival±s.d. of six replicates. Data representative of two independent experiments. (d) CAL-27 and LN229 cells treated with cetuximab or C-MMAE overnight, irradiated with 6 Gy, and comet tail length measured by neutral comet assay. Data normalized to vehicle treated, non-irradiated cells and plotted as mean relative comet tail length±s.e.m. of >50 cells per group. Data representative of two independent experiments. (e) CAL-27 and LN229 cells exposed to 2 nM Cy5-labelled C-MMAE for 2 h and Cy5 fluorescence (red) imaged. Nuclei stained with DAPI (blue). Scale bar, 10 μm. (f) CALU3 (EGFR+, HER2+) and OE19 cells treated with free MMAE, ErbB antibodies (cetuximab or trastuzumab) or ADC (C-MMAE or T-MMAE) overnight, irradiated with 6 Gy, and comet tail length measured using neutral comet assay. Comet tail length normalized to vehicle treated, non-irradiated cells and plotted as mean relative comet tail length s.e.m. of >50 cells per group. Data representative of two independent experiments. All statistical significances were calculated using one-way ANOVA with Tukey's multiple comparisons test. *P<0.05, **P<0.01, ***P<0.001. ****P<0.0001.

FIG. 3: Clinical grade anti-tubulin ADC T-DM1 radiosensitizes HER2+ tumours. (a) Clonogenic cell survival (SF2) of cells treated with 5 nM mertansine overnight followed by 2 Gy. Cell viability normalized to non-irradiated cells for each drug condition and plotted as mean fractional survival±s.d. of five replicates. Data representative of two independent experiments. (b) IC₅₀ of mertansine and T-DM1 in cells based on HER2 expression. Cells treated with a dose range of drug for 72-96 h and viability measured, IC₅₀ calculated and plotted as mean±s.d. of three independent experiments. (c) Cytotoxicity of clinically used radiosensitizers. OE19 cells were exposed to a dose range of drugs, viability normalized to vehicle treated cells and plotted as mean fractional survival±s.d. of three replicates. Data representative of three independent experiments (d) Cell cycle profile of OE19 and HCT116 cells treated with T-DM1 overnight, stained with propidium iodide and analysed by flow cytometry. Data representative of two independent experiments. (e) Neutral comet assay of HER2+ and HER2− cells treated with 20 nM mertansine, trastuzumab or T-DM1 overnight and irradiated with 2 Gy. Comet tail length was normalized to vehicle treated, non-irradiated cells and plotted as relative comet tail length s.e.m. of >50 cells per group. Data representative of two independent experiments. (f) Representative images of comet tails and quantification of relative comet tail length of OE19 cells treated with 0-5 nM of T-DM1 and irradiated. Comet tail length was normalized to vehicle treated, non-irradiated cells and plotted as relative comet tail length s.e.m. of >50 cells per group. Scale bar, 50 μm. All statistical significances were calculated using one-way ANOVA with Tukey's multiple comparisons test. *P<0.05, **P<0.01, ****P<0.0001.

FIG. 4: Antibody conjugated MMAE selectively targets tumours and increases efficiency of tumour regression in combination with IR. (a) OE19 tumour xenografts grown in the flank and bilateral thighs (location indicated by three red arrows) of mice and 0.5 nmol of Cy5-labelled C-MMAE or T-MMAE (B4 and B3.9 mgkg-1, respectively) intravenously injected. Tumours imaged 48 h later for Cy5 fluorescence (upper left). Yellow arrowheads indicate gut auto-fluorescence. Tumour xenografts were harvested and stained for pHistone H3 (bottom left). From T-MMAE injected mice, tumours were H&E stained and imaged for Cy5 fluorescence (right). Scale bar, 50 μm. Representative imaging of two mice per group. (b) Mice were injected with Cy5-labelled T-MMAE, blood collected, Cy5 fluorescence measured and plotted as mean±s.d. of four mice. (c) EGFR+ tumour xenografts grown in the bilateral thighs of athymic nude mice (left), 0.5 nmoles of Cy5-labelled C-MMAE (B4 mgkg-1) injected, 24 h later right tumour bearing thigh irradiated with 3 Gy and mice imaged 24 h post IR for Cy5 fluorescence. Non-irradiated, mice bearing unilateral CAL-27 tumour xenografts (right), 0.5 nmol of Cy5-labelled C-MMAE (B4 mgkg-1) injected and mice imaged at 48 and 72 h for Cy5 fluorescence. Representative imaging of three mice per group. (d) EGFR+ tumour-bearing mice were injected with cetuximab or C-MMAE, harvested 24 h later and stained for pS10 histone H3. Scale bar, 50 μm. Representative imaging of three mice per group. (e) CAL-27 tumour-bearing mice were injected with 2 nmol of C-MMAE or T-MMAE (B15.9 and B15.4 mg kg-1, respectively) and harvested. Tumour drug concentrations quantitated LC-MS/MS and plotted as mean±s.e.m. of four tumours. Statistical significance was calculated using one-way ANOVA with Tukey's multiple comparisons test. (f,g) Mice bearing CAL-27 tumours injected on day 0 with indicated drugs on day 0 and 3 Gy given on days 1, 2. Tumours were measured and plotted as mean tumour volume±s.e.m. of >8 tumours per group. Statistical significances were calculated using two-way ANOVA with Tukey's multiple comparisons test. **P<0.01, ****P<0.0001.

FIG. 5: T-DM1 combined with IR results in long-term HER2+ tumour control. (a) OE19 cells were exposed to trastuzumab, T-MMAE or T-DM1 for 96 h. Cell viability normalized to vehicle-treated cells and plotted as mean fractional survival±s.d. of three replicates. Data representative of three independent experiments. (b) Mice bearing OE19 tumour xenografts were i.v. injected on day 0 with 1 nmol trastuzumab, T-DM1 or T-MMAE (−7.3, −7.4 and −7.7 mg kg′, respectively). Tumours were measured twice a week and plotted as mean tumour volume±s.e.m. of 10 tumours per group. Statistical significances were calculated using two-way ANOVA with Tukey's multiple comparisons test. (c) Tumour from OE19 tumours harvested 48 h after trastuzumab or T-MMAE and stained for cleaved caspase 3 or p-Histone H3 by IHC. Data were normalized to untreated, control tumours and plotted as mean±s.e.m. of >3 replicates. Statistical significance was calculated using one-way ANOVA with Tukey's multiple comparisons test. Representative images of pHistone H3 shown. Scale bar, 75 gm. (d) Mice bearing OE19 or NCI N87 (HER2+) or HCT116 (HER2−) tumour xenografts were i.v. injected on day 0 with 0.25 nmol trastuzumab or T-DM1 (−1.8 and −1.9 mg kg′, respectively). IR-treated mice were given 2.5 Gy on days 1, 2 and 3. Tumours were measured twice a week and plotted as mean tumour volume±s.e.m. of >4 tumours per group. Statistical significances were calculated using two-way ANOVA with Tukey's multiple comparisons test. (e) Survival curve of HER2+ treated tumour xenografts of >2 mice per group. Statistical significances were calculated using log-rank (Mantel-Cox) test. *P<0.05, **P<0.01, ****P<0.0001.

FIG. 6: T-MMAF (Trastuzumab conjugated MMAF) and P-MMAF (Pertuzumab conjugated MMAF) selectively bind to HER2 expressing tumor cells. Cy5 labeled T-MMAF or P-MMAF exposed to HER2+(OE19 and NCI N87) or HER2− (HCT116) cells. Cy5 signal localized to HER2+ cells but not HER2− cells by direct confocal microscopy (Above) or flow cytometry (Right).

FIG. 7: T-MMAF (Trastuzumab conjugated MMAF) and P-MMAF (Pertuzumab conjugated MMAF) selectively kill HER2 expressing tumor cells. MMAF has decreased potency compared to MMAE across tumor cell lines. MMAF potency can be increased and cytotoxicity restricted by antibody conjugation. Cell survival measured by Alamar Blue assay.

FIG. 8: T-MMAF (Trastuzumab conjugated MMAF) and P-MMAF (Pertuzumab conjugated MMAF) radiosensitize HER2+ tumor cells. Neutral comet assay to measure DNA double strand breaks following 2 Gy of ionizing radiation (IR). IR increased comet tail length by 2.8 fold comapred to non-irradiated cells. Free MMAE but not MMAF further increased IR induced DNA damage. P-MMAF and T-MMAF also increased further increased IR induced DNA double strand breaks compared to vehicle, free MMAF or antibody alone (pertuzumab or trastuzumab). *p<0.05, **p<0.01, ****p<0.0001.

FIG. 9: TrastMMAF (Trastuzumab conjugated MMAF) and PertMMAF (Pertuzumab conjugated MMAF) preferentially deliver radiosensitizing MMAF to tumor cells and not surrounding normal muscle tissue. OE19 tumors (HER2+) were subcutaneously established in the flanks of nude mice. TrastMMAF or PertMMAF was injected intravenously. Tumor and surrounding muscle tissue were harvested on days 1, 2 and 7. MMAF drug concentration was measured using HPLC techniques. Pertuzumab conjugation resulted in increased radiosensitizer (MMAF) delivery compared to trastuzumab conjugation.

FIG. 10: P-MMAE (Pertuzumab conjugated MMAE) and P-MMAF (Pertuzumab conjugated MMAF) combined with ionizing radiation (IR) improves tumor control. OE19 tumors (HER2+) were subcutaneously established in the flanks of nude mice. Mice were intravenously injected with pertuzumab, P-MMAE or P-MMAF on day 0. IR was delivered on days 1 and 2. Tumors were measured directly with digital calipers. All Control, IR alone, Pertuzumab and Pertuz+IR treated mice has their tumors grow and were sacrificed due to tumor burden. In contrast, antibody conjugates of MMAE and MMAF resulted in long term tumor control that was further increased in combination with IR P-MMAE had more single agent efficacy compared to P-MMAF. P-MMAE with IR had increased mouse survival compared to P-MMAE. P-MMAF with IR had increased mouse survival compared to P-MMAF.

### SUMMARY OF THE INVENTION

In some embodiments, the present invention provides a method for inducing radiosensitization comprising administering to a subject in need thereof a molecule comprising an antibody conjugated to a radiosensitizing agent T, wherein said radiosensitizing agent is not an auristatin, including MMAE.

In some embodiments, the present invention provides method for inducing radiosensitization comprising administering to a subject in need thereof a molecule comprising the formula:

A-T

wherein the molecule comprises:

an antibody A;

a radiosensitizing agent T, wherein said radiosensitizing agent is not an auristatin, including MMAE,

wherein said subject is radiosensitized.

In some embodiments, T is a maytansinoid. In some embodiments, T is mertansine.

In some embodiments, the antibody is an anti-ErbB antibody. In some embodiments, the antibody is an anti-HER2 antibody. In some embodiments, the antibody is an anti-EGFR antibody.

In some embodiments, antibody is selected from the group consisting of cetuximab, trastuzumab, and pertruzumab.

In some embodiments, the molecule comprises the formula:

A-Y-T

wherein the molecule comprises:

a cleavable linker Y.

In some embodiments, Y is selected from the group consisting of Val-Cit-(p-amido)benzyloxycarbonyl and maleimidocaproyl-valine-citrulline-paminobenzyloxycarbonyl (MC-VC-PABC).

In some embodiments, the molecule is administered prior to administration of a radiation therapy.

In some embodiments, the molecule is administered concurrently with administration of a radiation therapy.

In some embodiments, the cleavable linker is cleaved in vivo in the presence of a tumor in the subject.

In some embodiments, the present invention provides a composition for inducing radiosensitization in a subject, wherein said composition comprises a molecule comprising the formula:

A-T

wherein the molecule comprises:

an antibody A;

a radiosensitizing agent T, wherein said radiosensitizing agent is not an auristatin, including MMAE.

In some embodiments, T is a maytansinoid. In some embodiments, T is mertansine.

In some embodiments, antibody is an anti-ErbB antibody. In some embodiments, the antibody is an anti-HER2 antibody. In some embodiments, the antibody is an anti-EGFR antibody.

In some embodiments, the antibody is selected from the group consisting of cetuximab, trastuzumab, and pertruzumab.

In some embodiments, the molecule comprises the formula:

A-Y-T

wherein the molecule comprises:

a cleavable linker Y.

In some embodiments, Y is selected from the group consisting of Val-Cit-(p-amido)benzyloxycarbonyl and maleimidocaproyl-valine-citrulline-paminobenzyloxycarbonyl (MC-VC-PABC).

In some embodiments, the composition comprises a pharmaceutically acceptable excipient.

In some embodiments, the molecule is administered prior to administration of a radiation therapy.

In some embodiments, the molecule is administered concurrently with administration of a radiation therapy.

In some embodiments, the cleavable linker is cleaved in vivo in the presence of a tumor in the subject.

In some embodiments, the present invention provides a composition for inducing tumor cell death in a subject, wherein said composition comprises a molecule comprising the formula:

A-T

wherein the molecule comprises:

an antibody A;

a radiosensitizing agent T, wherein said radiosensitizing agent is not an auristatin, including MMAE.

In some embodiments, T is a maytansinoid. In some embodiments, T is mertansine.

In some embodiments, antibody is an anti-ErbB antibody. In some embodiments, the antibody is an anti-HER2 antibody. In some embodiments, the antibody is an anti-EGFR antibody.

In some embodiments, the antibody is selected from the group consisting of cetuximab, trastuzumab, and pertruzumab.

In some embodiments, the molecule comprises the formula:

A-Y-T

wherein the molecule comprises:

a cleavable linker Y.

In some embodiments, Y is selected from the group consisting of Val-Cit-(p-amido)benzyloxycarbonyl and maleimidocaproyl-valine-citrulline-paminobenzyloxycarbonyl (MC-VC-PABC).

In some embodiments, the composition comprises a pharmaceutically acceptable excipient.

In some embodiments, the molecule is administered prior to administration of a radiation therapy.

In some embodiments, the molecule is administered concurrently with administration of a radiation therapy.

In some embodiments, the cleavable linker is cleaved in vivo in the presence of a tumor in the subject.

### BRIEF DESCRIPTION OF THE DRAWINGS

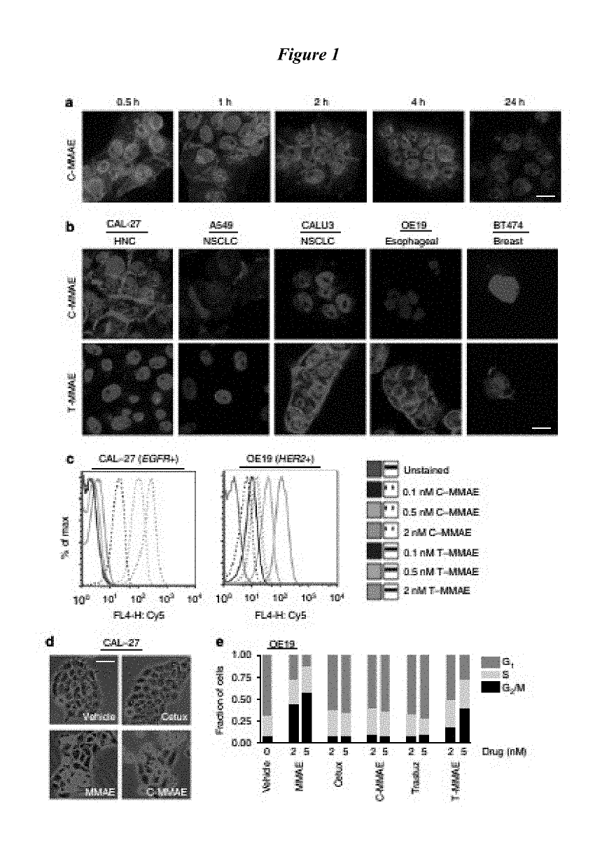

FIG. 1: Anti-ErbB antibody MMAE conjugates bind in a receptor dependent manner. a) CAL-27 (EGFR+) cells exposed to 2 nM of Cy5 labeled C-MMAE for 30 minutes then incubated in drug free media. Cells were fixed at indicated times and imaged for Cy5 fluorescence (red). Nuclei stained with DAPI (blue). Scale bar, 10 μM. Representative images of 3 independent experiments. b) A panel of EGFR (CAL27, A549, CALU3) and HER2 (CALU3, OE19, BT474) expressing cells from diverse tumor histologies were exposed to 2 nM Cy5 labeled C-MMAE or T-MMAE for 2 hours and Cy5 fluorescence (red) imaged. Nuclei stained with DAPI (blue). Scale bar, 10 μM. Representative images of 3 independent experiments. c) Cell surface binding of Cy5 labeled C-MMAE or T-MMAE. CAL-27 and OE19 cells incubated on ice with increasing concentrations of C-MMAE or T-MMAE. of Flow cytometry assessment of Cy5 signal. Representative data of 2 independent experiments. d) Phase contrast microscopy of CAL-27 cells treated with 2 nM MMAE, cetuximab, or C-MMAE overnight. Representative images of 3 independent experiments. Scale bar, 50 μM. e) Cell cycle profile of OE19 cells treated with MMAE, ErbB antibodies (cetuximab or trastuzumab) or ADC (C-MMAE or T-MMAE) overnight, stained with propidium iodide and analyzed by flow cytometry. Data representative of 2 independent experiments.

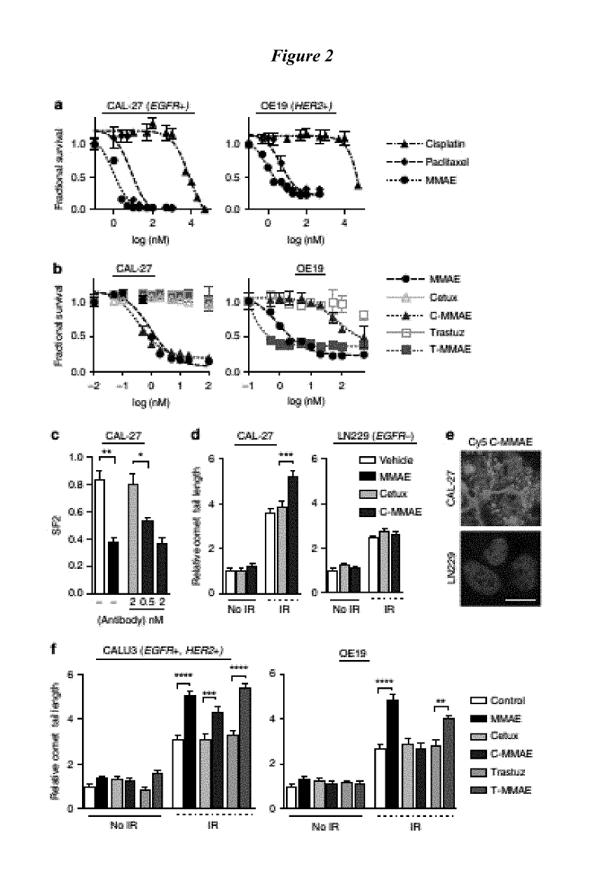

FIG. 2: Antibody conjugated MMAE selectively radiosensitizes tumor cells. a) CAL-27 (EGRF+) and OE19 (HER2+) cells were exposed to a dose range of cisplatin, paclitaxel or MMAE for 72-96 hours. Cell viability normalized to vehicle treated cells and plotted as mean fractional survival±SD of 3 replicates. Data representative of 3 independent experiments. b) CAL-27 and OE19 tumor cells were exposed to dose range of MMAE, ErbB antibody (cetuximab or trastuzumab) or ADC (C-MMAE or T-MMAE) for 72 hours. Cell viability plotted as mean fractional survival±SD of 3 replicates. Data representative of 3 independent experiments. c) Clonogenic cell survival (SF2) of CAL-27 cells treated with MMAE, cetuximab or C-MMAE overnight followed by 0 or 2 Gy. Cell viability normalized to non-irradiated cells for each drug condition and plotted as mean fractional survival±SD of 6 replicates. Data representative of 2 independent experiments. d) CAL-27 and LN229 cells treated with cetuximab or C-MMAE overnight, irradiated with 6 Gy, and comet tail length measured by neutral comet assay. Data normalized to vehicle treated, non-irradiated cells and plotted as mean relative comet tail length±SEM of >50 cells per group. Data representative of 2 independent experiments. e) CAL-27 and LN229 cells exposed to 2 nM Cy5 labeled C-MMAE for 2 hours and Cy5 fluorescence (red) imaged. Nuclei stained with DAPI (blue). Scale bar, 10 μM. f) CALU3 (EGFR+, HER2+) and OE19 cells treated with free MMAE, ErbB antibodies (cetuximab or trastuzumab) or ADC (C-MMAE or T-MMAE) overnight, irradiated with 6 Gy, and comet tail length measured using neutral comet assay. Comet tail length normalized to vehicle treated, non-irradiated cells and plotted as mean relative comet tail length±SEM of >50 cells per group. Data representative of 2 independent experiments. All statistical significances were calculated using one-way ANOVA with Tukey's multiple comparisons test. *P<0.05, **P<0.01, ***P<0.001. ****P<0.0001.

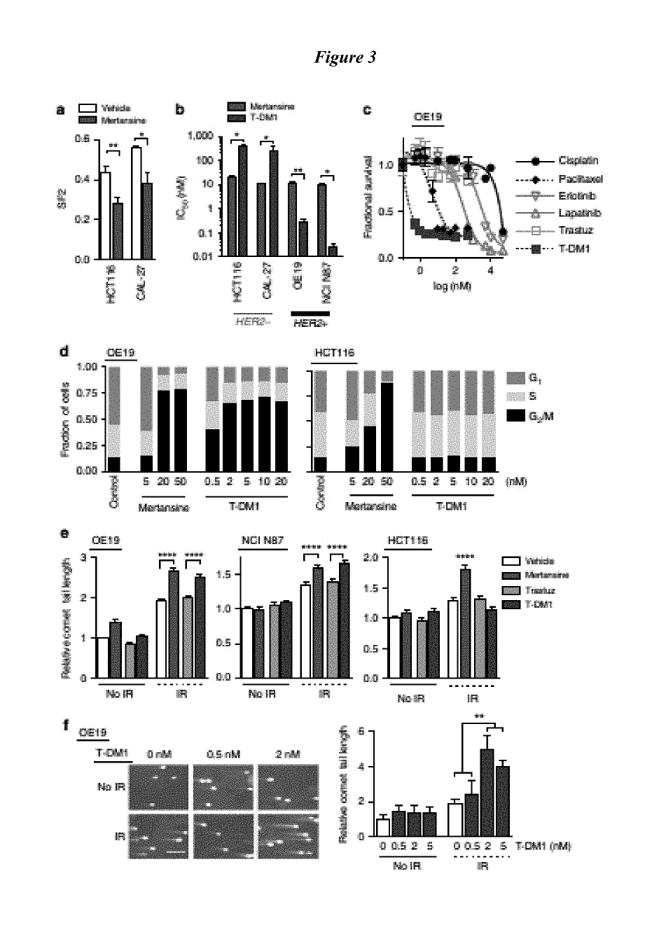

FIG. 3: Clinical grade anti-tubulin ADC T-DM1 radiosensitizes HER2+ tumors. a) Clonogenic cell survival (SF2) of cells treated with 5 nM mertansine overnight followed by 2 Gy. Cell viability normalized to non-irradiated cells for each drug condition and plotted as mean fractional survival±SD of 5 replicates. Data representative of 2 independent experiments. b) IC₅₀ of mertansine and T-DM1 in cells based on HER2 expression. Cells treated with a dose range of drug for 72-96 hr and viability measured, IC₅₀ calculated and plotted as mean±SD of 3 independent experiments. c) Cytotoxicity of clinically used radiosensitizers. OE19 cells were exposed to a dose range of drugs, viability normalized to vehicle treated cells and plotted as mean fractional survival±SD of 3 replicates. Data representative of 3 independent experiments d) Cell cycle profile of OE19 and HCT116 cells treated with T-DM1 overnight, stained with propidium iodide and analyzed by flow cytometry. Data representative of 2 independent experiments. e) Neutral comet assay of HER2+ and HER2− cells treated with 20 nM mertansine, trastuzumab or T-DM1 overnight and irradiated with 2 Gy. Comet tail length was normalized to vehicle treated, non-irradiated cells and plotted as relative comet tail length±SEM of >50 cells per group. Data representative of 2 independent experiments. f) Representative images of comet tails and quantification of relative comet tail length of OE19 cells treated with 0-5 nM of T-DM1 and irradiated. Comet tail length was normalized to vehicle treated, non-irradiated cells and plotted as relative comet tail length±SEM of >50 cells per group. Scale bar, 50 μM. All statistical significances were calculated using one-way ANOVA with Tukey's multiple comparisons test. *P<0.05, **P<0.01, **** P<0.0001.

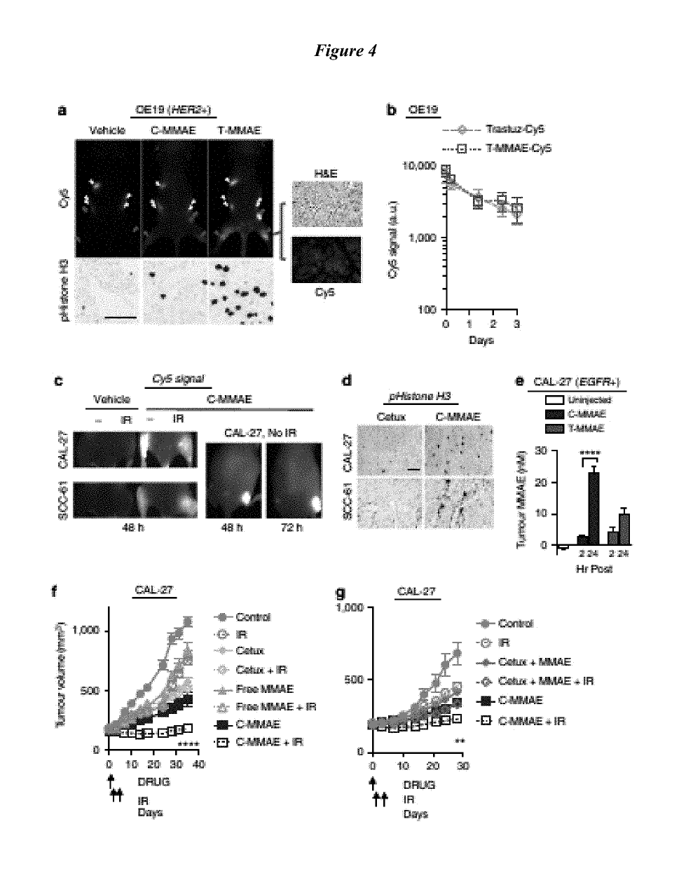

FIG. 4: Antibody conjugated MMAE selectively targets tumors and increases efficiency of tumor regression in combination with IR. a) OE19 tumor xenografts grown in the flank and bilateral thighs (location indicated by 3 red arrows) of mice and 0.5 nmoles of Cy5 labeled C-MMAE or T-MMAE (˜4 and ˜3.9 mg/kg respectively) intravenously injected. Tumors imaged 48 hours later for Cy5 fluorescence (upper left). Yellow arrowheads indicate gut auto-fluorescence. Tumor xenografts were harvested and stained for pHistone H3 (bottom left). From T-MMAE injected mice, tumors were H&E stained and imaged for Cy5 fluorescence (right). Scale bar, 50 μM. Representative imaging of 2 mice per group. b) Mice were injected with Cy5 labeled T-MMAE, blood collected, Cy5 fluorescence measured and plotted as mean±SD of 4 mice. c) EGFR+ tumor xenografts grown in the bilateral thighs of athymic nude mice (left), 0.5 nmoles of Cy5 labeled C-MMAE (˜4 mg/kg) injected, 24 hours later right tumor bearing thigh irradiated with 3 Gy and mice imaged 24 hours post IR for Cy5 fluorescence. Non-irradiated, mice bearing unilateral CAL-27 tumor xenografts (right), 0.5 nmoles of Cy5 labeled C-MMAE (˜4 mg/kg) injected and mice imaged at 48 and 72 hours for Cy5 fluorescence. Representative imaging of 3 mice per group. d) EGFR+ tumor bearing mice were injected with cetuximab or C-MMAE, harvested 24 hours later and stained for pS10 histone H3. Scale bar, 50 μM. Representative imaging of 3 mice per group. e) CAL-27 tumor bearing mice were injected with 2 nmole of C-MMAE or T-MMAE (˜15.9 and ˜15.4 mg/kg respectively) and harvested. Tumor drug concentrations quantitated LC-MS/MS and plotted as mean±SEM of 4 tumors. Statistical significance was calculated using one-way ANOVA with Tukey's multiple comparisons test. f, g) Mice bearing CAL-27 tumors injected on day 0 with indicated drugs on day 0 and 3 Gy given on days 1, 2. Tumors were measured and plotted as mean tumor volume±SEM of >8 tumors per group. Statistical significances were calculated using two-way ANOVA with Tukey's multiple comparisons test. **P<0.01, **** P<0.0001.

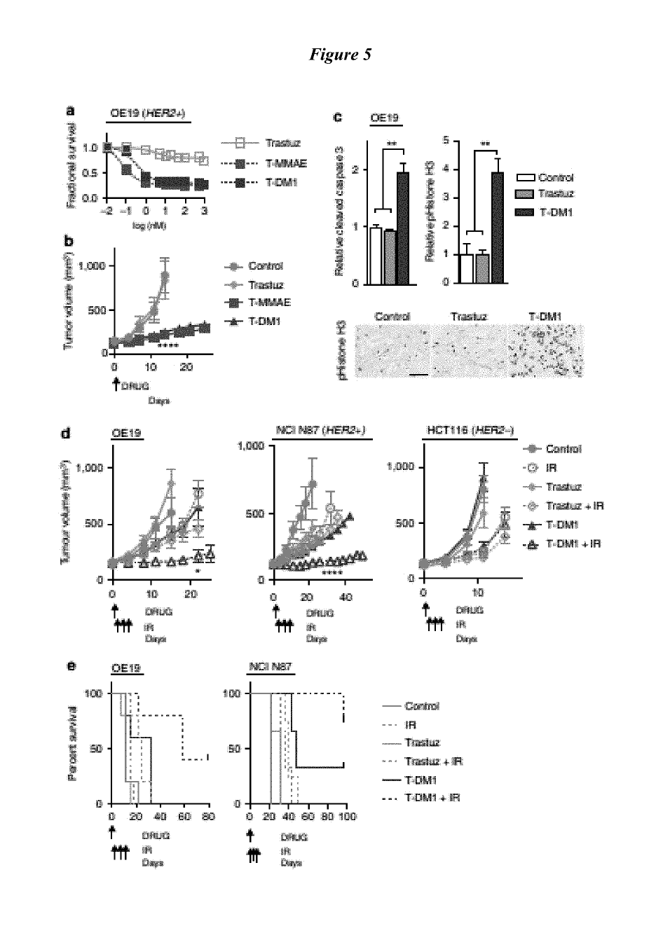

FIG. 5: T-DM1 combined with IR results in long term HER2+ tumor control. a) OE19 cells were exposed to trastuzumab, T-MMAE or T-DM1 for 96 hours. Cell viability normalized to vehicle treated cells and plotted as mean fractional survival±SD of 3 replicates. Data representative of 3 independent experiments. b) Mice bearing OE19 tumor xenografts were IV injected on day 0 with 1 nmole trastuzumab, T-DM1 or T-MMAE (˜7.3, ˜7.4 and ˜7.7 mg/kg respectively). Tumors were measured twice a week and plotted as mean tumor volume±SEM of 10 tumors per group. Statistical significances were calculated using two-way ANOVA with Tukey's multiple comparisons test. c) Tumor from OE19 tumors harvested 48 hours after trastuzumab or T-MMAE and stained for cleaved caspase 3 or p-Histone H3 by IHC. Data were normalized to untreated, control tumors and plotted as mean±SEM of >3 replicates. Statistical significance was calculated using one-way ANOVA with Tukey's multiple comparisons test. Representative images of pHistone H3 shown. Scale bar, 75 d) Mice bearing OE19 or NCI N87 (HER2+) or HCT116 (HER2-) tumor xenografts were IV injected on day 0 with 0.25 nmoles trastuzumab or T-DM1 (˜1.8 and ˜1.9 mg/kg respectively). IR treated mice were given 2.5 Gy on days 1, 2 and 3. Tumors were measured twice a week and plotted as mean tumor volume±SEM of >4 tumors per group. Statistical significances were calculated using two-way ANOVA with Tukey's multiple comparisons test. e) Survival curve of HER2+ treated tumor xenografts of >2 mice per group. Statistical significances were calculated using Log-rank (Mantel-Cox) test. *P<0.05, **P<0.01, ****P<0.0001

### DETAILED DESCRIPTION OF THE INVENTION

### Introduction

Activatable cell penetrating peptides (ACPPs) are peptide based molecules in which a polycationic sequence, typically comprising 8-12 arginines, is connected via an enzyme cleavable linker to a polyanionic sequence, typically comprising a matching number of glutamates. ACPP is described in greater detail in US 2012/0134922 as well as PCT/US2014/013687, herein incorporated by reference in their entireties.

The ACPP described herein includes a first cleavable linker X that separates the acidic peptide domain from the basic peptide domain. The second cleavable linker Y is a cleavable linker linked to a compound, such as a therapeutic agent, that is preferably an intracellularly cleavable linker. The position of the second cleavable linker can be positioned anywhere along the basic peptide domain, the interface between the first cleavable marker and the basic peptide domain, or on the first cleavable linker. If the second cleavable linker is positioned on the first cleavable linker, it should be positioned such that following cleavage of the first cleavable linker, the second cleavable linker and its associated cargo remains associated with the basic peptide domain to allow delivery of the cargo into the cell, and does not interfere with the cleavage of the first cleavable linker.

Disclosed is a new sub class of ACPP that accommodates 1) pretargeting agent/ligand, 2) prodrug attached by a linker cleavable after endocytosis, and 3) contrast or imaging agent. This allows ACPP to be used as a molecular dual targeted theranostic agent. The pre-targeted ACPP embodiments described herein synergistically enhance tumor contrast, reduce tumor growth, and enhance the overall survival rate in a patient in need thereof.

The present invention is also based in part on the discovery that ex vivo cleavage of ratiometric MTSs (ACPPs) by tumor extract correlates with in-vivo MTS (ACPP) fluorescence uptake and increased emission ratio in cancer, particularly carcinoma. In some embodiments, measuring the ability of individual tumors to cleave MTSs (ACPPs) and assessing the percentage of enzymatically positive tumors in a clinical population provides valuable data in that the ex vivo cleavage data can be correlated with MTS (ACPP) performance in vivo. In some embodiments, the ex vivo cleavage assay may be further developed into a personalized screening assay to determine eligibility to use MTSs (ACPPs) during a given patient procedure such as for example surgery. In some embodiments, the present invention provides methods for assessing the distribution of human surgical specimens with respect to their ability to cleave the MTSs (ACPPs) and the correlation of the MTS with clinical grade and outcome. Methods and compositions useful in such methods are provided herein.

### Certain Definitions

The following terms have the meanings ascribed to them unless specified otherwise.

The terms cell penetrating peptide (CPP), activatable cell penetrating peptide (ACPP), membrane translocating sequence (MTS) and protein transduction domain are used interchangeably. As used herein, the terms mean a peptide (polypeptide or protein) sequence that is able to translocate across the plasma membrane of a cell. In some embodiments, a CPP facilitates the translocation of an extracellular molecule across the plasma membrane of a cell. In some embodiments, the CPP translocates across the plasma membrane by direct penetration of the plasma membrane, endocytosis-mediated entry, or the formation of a transitory structure. In some embodiments the MTS is not transported across the membrane of a cell, but is employed in an ex vivo assay or application.

As used herein, the term “aptamer” refers to a DNA or RNA molecule that has been selected from random pools based on their ability to bind other molecules with high affinity specificity based on non-Watson and Crick interactions with the target molecule (see, e.g., Cox and Ellington, Bioorg. Med. Chem. 9:2525-2531 (2001); Lee et al., Nuc. Acids Res. 32:D95-D100 (2004)). In some embodiments, the aptamer binds nucleic acids, proteins, small organic compounds, vitamins, inorganic compounds, cells, and even entire organisms.

The terms “polypeptide,” “peptide” and “protein” and derivatives thereof as used herein, are used interchangeably herein to refer to a polymer of amino acid residues. The terms apply to naturally occurring amino acid polymers as well as amino acid polymers in which one or more amino acid residues is a non-naturally occurring amino acid (e.g., an amino acid analog). The terms encompass amino acid chains of any length, including full length proteins (i.e., antigens), wherein the amino acid residues are linked by covalent peptide bonds. As used herein, the terms “peptide” refers to a polymer of amino acid residues typically ranging in length from 2 to about 50 residues. In certain embodiments the peptide ranges in length from about 2, 3, 4, 5, 7, 9, 10, or 11 residues to about 50, 45, 40, 45, 30, 25, 20, or 15 residues. In certain embodiments the peptide ranges in length from about 8, 9, 10, 11, or 12 residues to about 15, 20 or 25 residues. Where an amino acid sequence is provided herein, L-, D-, or beta amino acid versions of the sequence are also contemplated as well as retro, inversion, and retro-inversion isoforms. Peptides also include amino acid polymers in which one or more amino acid residues is an artificial chemical analogue of a corresponding naturally occurring amino acid, as well as to naturally occurring amino acid polymers. In addition, the term applies to amino acids joined by a peptide linkage or by other modified linkages (e.g., where the peptide bond is replaced by an α-ester, a β-ester, a thioamide, phosphonamide, carbamate, hydroxylate, and the like (see, e.g., Spatola, Chem. Biochem. Amino Acids and Proteins 7: 267-357 (1983)), where the amide is replaced with a saturated amine (see, e.g., Skiles et al., U.S. Pat. No. 4,496,542, which is incorporated herein by reference, and Kaltenbronn et al., (1990) Pp. 969-970 in Proc. 11th American Peptide Symposium, ESCOM Science Publishers, The Netherlands, and the like)).

The term “amino acid” and derivatives thereof as used herein, refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. Amino acids may be either D amino acids or L amino acids. In peptide sequences throughout the specification, lower case letters indicate the D isomer of the amino acid (conversely, upper case letters indicate the L isomer of the amino acid).

Amino acids may be referred to herein by either their commonly known three letter symbols or by the one-letter symbols recommended by the IUPAC-IUB Biochemical Nomenclature Commission.

Nucleotides, likewise, may be referred to by their commonly accepted single-letter codes.

One of skill will recognize that individual substitutions, deletions or additions to a peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

As used herein, a “linker” or “spacer” is any molecule capable of binding (e.g., covalently) portions an MTS molecule as disclosed herein together. Linkers include, but are not limited to, straight or branched chain carbon linkers, heterocyclic carbon linkers, peptide linkers, polyether linkers and short hydrophilic molecules. Exemplary linkers can include but are not limited to NH—CH₂—CH₂—O—CH₂—CO— and 5-amino-3-oxopentanoyl. For example, poly(ethylene glycol) linkers are available from Quanta Biodesign, Powell, Ohio These linkers optionally have amide linkages, sulfhydryl linkages, or heterofunctional linkages.

As used herein, the term “label” refers to any molecule that facilitates the visualization and/or detection of a MTS molecule disclosed herein. In some embodiments, the label is a fluorescent moiety.

The term “carrier” or “macromolecular carrier” means an inert molecule that increases (a) plasma half-life and (b) solubility. In some embodiments, a carrier increases plasma half-life and solubility by reducing glomerular filtration. In some embodiments, a carrier increases tumor uptake due to enhanced permeability and retention (EPR) of tumor vasculature. Exemplary macromolecular carriers include but are not limited to dendrimers, dextrans, PEG polymers, albumins, or lipid-coated perfluorocarbon droplets.

The term “thrombin” means an enzyme (EC 3.4.21.5) that cleaves fibrinogen molecules into fibrin monomers. Thrombin, acting through its G-protein coupled receptor PAR-I, is a key player in a wide range of vascular and extravascular disease processes throughout the body, including cancer, cardiovascular diseases, acute kidney injury, and stroke. In certain instances, thrombin activity increases over the course of atherosclerotic plaque development. In some embodiments, thrombin activity is a biomarker for atherosclerotic plaque development.

The term “reactive oxygen species” or “ROS” includes peroxide compounds or compounds with peroxide activity. Examples include but are not limited to hydrogen peroxide. Hydrogen peroxide is represented by the formula H₂O₂. Hydrogen peroxide is commonly found endogenously in living organisms. H₂O₂ plays an active role in the regulation of various physiological processes; however, its overabundance results in oxidative stress that can lead to extensive cellular damage. Indeed, high levels of H₂O₂ have been implicated in many pathological conditions including inflammation, diabetes, cardiovascular diseases, neurodegenerative disorders and cancer.

The terms “individual,” “patient,” or “subject” are used interchangeably. As used herein, they mean any mammal (i.e., species of any orders, families, and genus within the taxonomic classification animalia: chordata: vertebrata: mammalia). In some embodiments, the mammal is a human. None of the terms require or are limited to situation characterized by the supervision (e.g., constant or intermittent) of a health care worker (e.g., a doctor, a registered nurse, a nurse practitioner, a physician's assistant, an orderly, or a hospice worker).

As used herein, the term “medical professional” means any health care worker. By way of non-limiting example, the health care worker may be a doctor, a registered nurse, a nurse practitioner, a physician's assistant, an orderly, or a hospice worker.

The terms “administer,” “administering”, “administration,” and derivatives thereof as used herein, refer to the methods that may be used to enable delivery of agents or compositions to the desired site of biological action These methods include, but are not limited to parenteral injection (e.g., intravenous, subcutaneous, intraperitoneal, intramuscular, intravascular, intrathecal, intravitreal, infusion, or local) Administration techniques that are optionally employed with the agents and methods described herein, include e.g., as discussed in Goodman and Gilman, The Pharmacological Basis of Therapeutics, current edition, Pergamon, and Remington's, Pharmaceutical Sciences (current edition), Mack Publishing Co, Easton, Pa.

The terms “treatment”, “treating”, “treat”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development or progression; and (c) relieving the disease, i.e., causing regression of the disease and/or relieving one or more disease symptoms. “Treatment” is also meant to encompass delivery of an MTS or other moleucle in order to provide for a pharmacologic effect, even in the absence of a disease or condition.

The term “effective amount” or “therapeutically effective amount” refers to that amount of a compound or combination of compounds as described herein (e.g., MTS molecules) that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, or the manner of administration. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

The term “in vivo” refers to an event that takes place in a subject's body.

The term “in vitro” refers to an event that takes places outside of a subject's body.

The term “pharmaceutically acceptable” and derivatives thereof as used herein, refers to a material that does not abrogate the biological activity or properties of the agents described herein, and is relatively nontoxic (i.e., the toxicity of the material significantly outweighs the benefit of the material) In some instances, a pharmaceutically acceptable material may be administered to an individual without causing significant undesirable biological effects or significantly interacting in a deleterious manner with any of the components of the composition in which it is contained.

The terms “co-administration,” “co-administering,” “administered in combination with,” “administering in combination with,” “simultaneous,” and “concurrent,” as used herein, encompass administration of two or more active pharmaceutical ingredients to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present are included.

The term “surgery” and derivatives thereof as used herein, refers to any methods for that may be used to manipulate, change, or cause an effect by a physical intervention These methods include, but are not limited to open surgery, endoscopic surgery, laparoscopic surgery, minimally invasive surgery, and robotic surgery.

The terms “neoplasm” or “neoplasia” and derivatives thereof as used herein, include any non-normal or non-standard cellular growth. Neoplasms can include tumors and cancers of any variety of stages, from benign to metastatic. Neoplasms can be primary or metastatic growths and can occur anywhere in a subject. Neoplasms can include neoplasms of the lung, skin, lymph, brain, nerves, muscle, breast, prostate, testis, pancreases, liver, kidneys, stomach, muscle, gastrointestinal, bone and blood. Neoplasms can be solid and non-solid tumors. In some embodiments, the neoplasm (e.g., tumor) expresses HER2. In some embodiments, the neoplasm (e.g., tumor) expresses ErbB. In some embodiments, the neoplasm (e.g., tumor), expresses EGFR.

The terms “sample” or “samples” and derivatives thereof as used herein, include any samples obtained from a subject with can be employed with the methods described herein. Samples can include but are not limited to urine, blood, lymph, tears, mucus, saliva, biopsy or other sample tissue samples. Sample can be frozen, refrigerated, previously frozen, and/or stored for minutes, hours, days, weeks, months, years. Sampling techniques, handling and storage are well known and any such techniques for obtaining samples for use with the present invention are contemplated.

The following symbols, where used, are used with the indicated meanings Fl=fluorescein, aca=ahx=X=ammohexanoyl linker (—HN—(CH₂)₅CO-)aminohexanoyl, C=L-cysteine, E=L-glutamate, R=L-arginine, D=L-aspartate, K=L-lysine, A=L-alanine, r=D-arginine, c=D-cysteine, e=D-glutamate, P=L-proline, L=L-leucine, G=glycine, V=valine, I=isoleucine, M=methionine, F=phenylalanine, Y=tyrosine, W=tryptophan, H=histidine, Q=glutamine, N=asparagine, S=serine, T=threonine, o is 5-amino-3-oxapentanoyl linker, and C(me) is S-methylcysteine.

The term “monoclonal antibody” as used herein refers to an antibody from a population of substantially homogeneous antibodies, i.e., the individual antibodies comprising the population are substantially similar and bind the same epitope(s), except for possible variants that may arise during production of the monoclonal antibody, such variants generally being present in minor amounts. Such monoclonal antibody typically includes an antibody comprising a variable region that binds a target, wherein the antibody was obtained by a process that includes the selection of the antibody from a plurality of antibodies. For example, the selection process can be the selection of a unique clone from a plurality of clones, such as a pool of hybridoma clones, phage clones or recombinant DNA clones. It should be understood that the selected antibody can be further altered, for example, to improve affinity for the target, to humanize the antibody, to improve its production in cell culture, to reduce its immunogenicity in vivo, to create a multispecific antibody, etc., and that an antibody comprising the altered variable region sequence is also a monoclonal antibody of this invention. In addition to their specificity, the monoclonal antibody preparations are advantageous in that they are typically uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including the hybridoma method (e.g., Kohler et al., Nature, 256:495 (1975); Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681, (Elsevier, N.Y., 1981), recombinant DNA methods (see, e.g., U.S. Pat. No. 4,816,567), phage display technologies (see, e.g., Clackson et al., Nature, 352:624-628 (1991); Marks et al., J. Mol. Biol., 222:581-597 (1991); Sidhu et al., J. Mol. Biol. 338(2):299-310 (2004); Lee et al., J. Mol. Biol. 340(5):1073-1093 (2004); Fellouse, Proc. Nat. Acad. Sci. USA 101(34):12467-12472 (2004); and Lee et al. J. Immunol. Methods 284(1-2):119-132 (2004) and technologies for producing human or human-like antibodies from animals that have parts or all of the human immunoglobulin loci or genes encoding human immunoglobulin sequences (see, e.g., WO98/24893, WO/9634096, WO/9633735, and WO/9110741, Jakobovits et al., Proc. Natl. Acad. Sci. USA, 90:2551 (1993); Jakobovits et al., Nature, 362:255-258 (1993); Bruggemann et al., Year in Immuno., 7:33 (1993); U.S. Pat. Nos. 5,545,806, 5,569,825, 5,591,669 (all of GenPharm); U.S. Pat. No. 5,545,807; WO 97/17852, U.S. Pat. Nos. 5,545,807; 5,545,806; 5,569,825; 5,625,126; 5,633,425; and 5,661,016, and Marks et al., Bio/Technology, 10: 779-783 (1992); Lonberg et al., Nature, 368: 856-859 (1994); Morrison, Nature, 368: 812-813 (1994); Fishwild et al., Nature Biotechnology, 14: 845-851 (1996); Neuberger, Nature Biotechnology, 14: 826 (1996); and Lonberg and Huszar, Intem. Rev. Immunol., 13: 65-93 (1995). In some embodiments, the antibody as described herein is a monoclonal antibody.

An “intact” antibody is one which comprises an antigen-binding site as well as a CL and at least heavy chain constant domains, C_{H1}, C_{H2}, and C_{H3}. The constant domains can be native sequence constant domains (e.g., human native sequence constant domains) or amino acid sequence variant thereof.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (see U.S. Pat. No. 5,641,870, Example 2; Zapata et al., Protein Eng. 8(10): 1057-1062 (1995)); single-chain antibody molecules; and multispecific antibodies formed from antibody fragments. The expression “linear antibodies” generally refers to the antibodies described in Zapata et al., Protein Eng., 8(10):1057-1062 (1995). Briefly, these antibodies comprise a pair of tandem Fd segments (V_{H}-C_{H1}-V_{H}-C_{H1}) which, together with complementary light chain polypeptides, form a pair of antigen binding regions. Linear antibodies can be bispecific or monospecific.

The terms “EGFR” and “anti-EGFR antibody” and variants thereof, refer generally to human epidermal growth factor receptor (also known as HER-1 or Erb-B1, and referred to herein as “EGFR”) is a 170 kDa transmembrane receptor encoded by the c-erbB proto-oncogene, and exhibits intrinsic tyrosine kinase activity (Modjtahedi, H., et al., Br. J. Cancer 73 (1996) 228-235; Herbst, R. S., and Shin, D. M., Cancer 94 (2002) 1593-1611) and antibodies to that receptor. SwissProt database entry P00533 provides the sequence of EGFR. There are also isoforms and variants of EGFR (e.g., alternative RNA transcripts, truncated versions, polymorphisms, etc.) including but not limited to those identified by Swissprot database entry numbers P00533-1, P00533-2, P00533-3, and P00533-4. EGFR is known to bind ligands including epidermal growth factor (EGF), transforming growth factor-α (TGF-α), amphiregulin, heparin-binding EGF (hb-EGF), betacellulin, and epiregulin (Herbst, R. S., and Shin, D. M., Cancer 94 (2002) 1593-1611; Mendelsohn, J., and Baselga, J., Oncogene 19 (2000) 6550-6565). EGFR regulates numerous cellular processes via tyrosine-kinase mediated signal transduction pathways, including, but not limited to, activation of signal transduction pathways that control cell proliferation, differentiation, cell survival, apoptosis, angiogenesis, mitogenesis, and metastasis (Atalay, G., et al., Ann. Oncology 14 (2003) 1346-1363; Tsao, A. S., and Herbst, R. S., Signal 4 (2003) 4-9; Herbst, R. S., and Shin, D. M., Cancer 94 (2002) 1593-1611; Modjtahedi, H., et al., Br. J. Cancer 73 (1996) 228-235).

The term “HER2” (also “ErbB2)”, and “anti-HER2 antibody” (also anti-ErbB2) and variants thereof, refer generally refers to 185 kDa growth factor receptor also referred to as neu and c-erbB-2 (Slamon, et al., Science 235 (1987) 177-182; Swiss-Prot P04626) whose function is related to neoplastic transformation in human breast cancer cells and antibodies to the receptor. The HER receptor will generally comprise an extracellular domain, which may bind an HER ligand; transmembrane domain, a conserved intracellular tyrosine kinase domain, and a carboxyl-terminal signaling domain harboring several tyrosine residues which can be phosphorylated. The extracellular domain of HER2 comprises four domains, Domain I (amino acid residues from about 1-195), Domain II (amino acid residues from about 196-320), Domain III (amino acid residues from about 321 488), and Domain IV (amino acid residues from about 489-632) (residue numbering without signal peptide). See Garrett, et al., Mol. Cell. 11 (2003) 495-505, Cho; et al., Nature 421 (2003) 756-760, Franklin, et al., Cancer Cell 5 (2004) 317-328, or Plowman. et al., Proc. Natl Acad. Sci. 90 (1.993) 1746-1750 and WO 2006/007398.

The term “trastuzumab”, sold as Herceptin®, as used herein refers to a recombinant humanized anti-HER2 monoclonal antibody used for the treatment of HER2 over-expressed/HER2 gene amplified metastatic breast cancer. Trastuzumab binds specifically to the same epitope of HER2 as the murine anti-HER2 antibody 4D5 described in Hudziak, et al., Mol. Cell. Biol. 9 (1989) 1165-1.172. Trastuzumab is a recombinant humanized version of the murine anti-HER2 antibody 4D5, referred to as rhuMAb 4D5 or trastuzumab) and has been clinically active in patients with HER2-over expressing metastatic breast cancers that had received extensive prior anticancer therapy. (Baselga, et al, J. Clin. Oncol. 14 (19%) 737-744). Trastuzumab and its method of preparation are described in U.S. Pat. No. 5,821,337.

The term “pertuzumab”, sold as Omnitarg®, as used herein refers to a recombinant humanized anti-HER2 monoclonal antibody used for the treatment of HER2 positive cancers. Pertuzumab hinds specifically to the 2C4 epitope, a different epitope on the extracellular domain of HER2 as trastuzumab. Pertuzurnab is the first in a new class of HER dimerisation inhibitors (HDIs). Through its binding to the HER2 extracellular domain, pertuzumab blocks ligand-activated heterodimerisation of HER2 with other HER family members. thereby inhibiting downstream signalling pathways and cellular processes associated with tumour growth and progression (Franklin, M. C., et al. Cancer Cell 5 (2004) 317-328 and Friess, T. et al Clin Cancer Res 11 (2005) 5300-5309). Pertuzumab is a recombinant humanized version of the murine anti-HER2 antibody 2C4 (referred to as rhuMAb 2C4 or pertuzumab) and it is described together with the respective method of preparation. in WO 01/00245 and WO 2006/007398.

The term “cetuximab”, sold as Erbitux™, as used herein refers to a recombinant, human/mouse chimeric monoclonal antibody that binds specifically to the extracellular domain of the human (EGFR). Cetuximab is composed of the Fb regions of a murine anti-EGFR antibody with human IgG1 heavy and kappa light chain constant regions and has an approximate molecular weight of 152 kDa. Cetuximab is produced in mammalian cell culture (murine myeloma). Erbitux is approved for the treatment of patients with metastatic colorectal cancer and whose tumor expresses EGFR. Cetuximab is described together with the respective method of preparation in, for example, U.S. Pat. No. 6,217,866.

### MTS Peptides

In some embodiments, a generic structure for peptides having features of the invention are selected from the following:

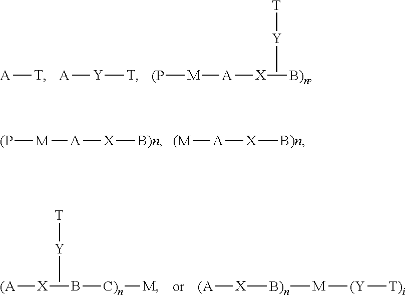

where P is an optional pre-targeting moiety; M is an optional macromolecular carrier; T is a compound for delivery to a target, including for example a therapeutic compound (also referred to herein as a “warhead”); peptide portion B includes between about 5 to about 20 basic amino acids; X is a cleavable linker portion, in some embodiments cleavable under physiological conditions; Y is a cleavable linker portion, in some embodiments cleavable under physiological conditions; and wherein peptide portion A includes between about 2 to about 20 acidic amino acids. In some embodiments, A is an antibody. In some embodiments, A is an anti-ErbB antibody In some embodiments, A is an anti-HER2 antibody. In some embodiments, the antibody is an anti-EGFR antibody. In some embodiments, A is an antibody selected from the group consisting of cetuximab and trastuzumab. In some embodiments of molecules having features of the invention, n and i are independently selected integers between 1 and 20. In some embodiments of molecules having features of the invention, n is an integer between 1 and 20. In some embodiments of molecules having features of the invention, n is an integer between 1 and 15. In some embodiments of molecules having features of the invention, n is an integer between 1 and 10. In some embodiments of molecules having features of the invention, n is an integer between 1 and 5. In some embodiments of molecules having features of the invention, n is an integer between 1 and 4. In some embodiments of molecules having features of the invention, n is an integer between 1 and 3. In some embodiments of molecules having features of the invention, n is an integer between 1 and 2. In some embodiments of molecules having features of the invention, n is 1. In some embodiments of molecules having features of the invention, n is an integer of 1, 2, 3, 4, 5, 6, 7, 8, 9, 10, 11, 12, 13, 14, 15, 16, 17, 18, 19, or 20. In some embodiments of molecules having features of the invention, i is an integer between 1 and 20. In some embodiments of molecules having features of the invention, i is an integer between 1 and 10. In some embodiments of molecules having features of the invention, i is an integer between 1 and 5. In some embodiments of molecules having features of the invention, i is an integer between 1 and 4. In some embodiments of molecules having features of the invention, i is an integer between 1 and 3. In some embodiments of molecules having features of the invention, i is an integer between 1 and 2. In some embodiments of molecules having features of the invention, i is 1. In some embodiments of molecules having features of the invention, i is an integer of 1, 2, 3, 4, 5, 6, 7, 8, 9, 10, 11, 12, 13, 14, 15, 16, 17, 18, 19, or 20. In some embodiments, the T-Y pair is attached to either end of B.

In some embodiments of molecules having features of the invention, peptide portion B includes between about 5 to about 20, or between about 9 to about 16 basic amino acids, and may be a series of basic amino acids (e.g., arginines, histidines, lysines, or other basic amino acids). In some embodiments of molecules having features of the invention, peptide portion A includes between about 2 to about 20, or between about 5 to about 20 acidic amino acids, and may be series of acidic amino acids (e.g., glutamates and aspartates or other acidic amino acids). A schematic representation of a MTS molecule having features of the invention comprising a basic portion B, a linker portion X, and an acidic portion A is presented in FIG. 1A of WO 2005/042034. In embodiments, MTS molecules having features of the invention may be cyclic molecules, as schematically illustrated in FIG. 1B of WO 2005/04203. Thus, MTS molecules having features of the invention may be linear molecules, cyclic molecules, or may be linear molecules including a cyclic portion.

In some embodiments, A is a peptide with a sequence comprising 5 to 9 consecutive acidic amino acids, wherein the amino acids are selected from: aspartates and glutamates; B is a peptide with a sequence comprising 5 to 20 consecutive basic amino acids; and X is a linker that is cleavable by a protease.

In some embodiments, A is a peptide with a sequence comprising 5 to 9 consecutive acidic amino acids, wherein the amino acids are selected from: aspartates and glutamates; B is a peptide with a sequence comprising 5 to 20 consecutive basic amino acids; X is a linker that is cleavable by thrombin; and M is a macromolecular carrier.

In some embodiments, A is an antibody. In some embodiments, A is an anti-ErbB antibody. In some embodiments, A is an anti-HER2 antibody. In some embodiments, the antibody is an anti-EGFR antibody. In some embodiments, A is an antibody selected from the group consisting of cetuximab, trastuzumab, and pertruzumab. In some embodiments, A is cetuximab. In some embodiments, A is trastuzumab. In some embodiments, A is pertruzumab.

Regulation of transport into and out of a cell is important for its continued viability. For example, cell membranes contain ion channels, pumps, and exchangers capable of facilitating the transmembrane passage of many important substances. However, transmembrane transport is selective in addition to facilitating the entry of desired substances into a cell, and facilitating the exit of others, a major role of a cell membrane is to prevent uncontrolled entry of substances into the cell interior. This barrier function of the cell membrane makes difficult the delivery of markers, drugs, nucleic acids, and other exogenous material into cells.

As discussed above, molecules including a multiple basic amino acids, such as a series of basic amino acids, are often taken up by cells. However, the present inventors have discovered that molecules having structures including a basic portion B, a linker portion X and/or Y and an acidic portion A are not taken up by cells. An acidic portion A may include amino acids that are not acidic. Acidic portion A may comprise other moieties, such as negatively charged moieties. In some embodiments acidic portion A does not include an amino acid. In embodiments of MTS molecules having features of the present disclosure, an acidic portion A may be a negatively charged portion, in some embodiments having about 2 to about 20 negative charges at physiological pH. A basic portion B may include amino acids that are not basic. Basic portion B may comprise other moieties, such as positively charged moieties. In some embodiments basic portion B does not include an amino acid. In embodiments of MTS molecules having features of the invention, a basic portion B may be a positively charged portion, having between about 5 and about 20 positive charges at physiological pH. Including an acidic portion A is effective to inhibit or prevent the uptake of a portion B into cells. Such a block of uptake that would otherwise be effected by the basic amino acids of portion B may be termed a “veto” of the uptake by the acidic portion A. The present inventors have made the further surprising discovery that cleavage of linker X, allowing the separation of portion A from portion B is effective to allow the uptake of portion B into cells.

In a some embodiments, MTS molecules of the present disclosure include the following:

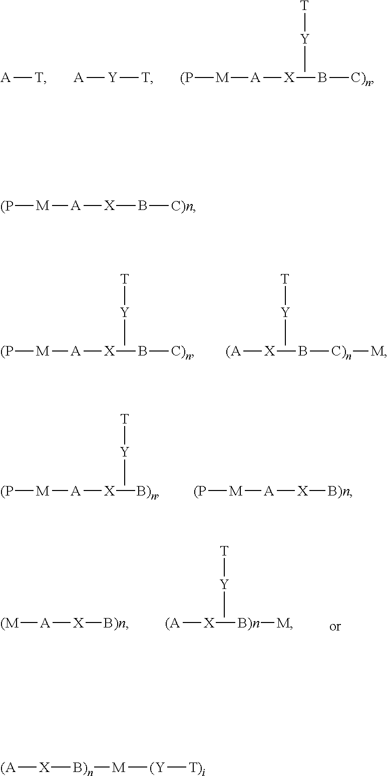

wherein P is an optional pre-targeting moiety; M is an optional macromolecular carrier; T is a compound for delivery to a target, including for example a therapeutic compound (also referred to herein as a “warhead”); A is peptide portion with a sequence comprising about 2 to about 20 acidic amino acids, which when linked with portion B is effective to inhibit or prevent cellular uptake of portion B; B is a peptide portion of about 5 to about 20 basic amino acid residues, which is suitable for cellular uptake; X is a first cleavable linker of about 2 to about 100 atoms, or about 3 to about 30 atoms, joining A with B; Y is a second cleavable linker of about 2 to about 100 atoms, or about 3 to about 30 atoms, joining A-X-B with T; T is a compound for delivery to a target, including for example a therapeutic compound (also referred to herein as a “warhead”); and n is an integer between 1 and 20; and C is a detectable cargo moiety. In some embodiments of molecules having features of the invention, n and i are independently selected integers between 1 and 20. In some embodiments of molecules having features of the invention, n is an integer between 1 and 20. In some embodiments of molecules having features of the invention, n is an integer between 1 and 15. In some embodiments of molecules having features of the invention, n is an integer between 1 and 10. In some embodiments of molecules having features of the invention, n is an integer between 1 and 5. In some embodiments of molecules having features of the invention, n is an integer between 1 and 4. In some embodiments of molecules having features of the invention, n is an integer between 1 and 3. In some embodiments of molecules having features of the invention, n is an integer between 1 and 2. In some embodiments of molecules having features of the invention, n is 1. In some embodiments of molecules having features of the invention, n is an integer of 1, 2, 3, 4, 5, 6, 7, 8, 9, 10, 11, 12, 13, 14, 15, 16, 17, 18, 19, or 20. In some embodiments of molecules having features of the invention, i is an integer between 1 and 20. In some embodiments of molecules having features of the invention, i is an integer between 1 and 10. In some embodiments of molecules having features of the invention, i is an integer between 1 and 5. In some embodiments of molecules having features of the invention, i is an integer between 1 and 4. In some embodiments of molecules having features of the invention, i is an integer between 1 and 3. In some embodiments of molecules having features of the invention, i is an integer between 1 and 2. In some embodiments of molecules having features of the invention, i is 1. In some embodiments of molecules having features of the invention, i is an integer of 1, 2, 3, 4, 5, 6, 7, 8, 9, 10, 11, 12, 13, 14, 15, 16, 17, 18, 19, or 20. An acidic portion A may include amino acids that are not acidic. In some embodiments, A-X-B is joined to T with linker Y. Acidic portion A may comprise other moieties, such as negatively charged moieties. In some embodiments, cleavage of linker X leads to B and T remaining joined together. In some embodiments, B and T remained joined together until cleavage of linker Y occurs. In embodiments of MTS molecules having features of the invention, an acidic portion A may be a negatively charged portion, having about 2 to about 20 negative charges at physiological pH that does not include an amino acid. A basic portion B may include amino acids that are not basic. Basic portion B may comprise other moieties, such as positively charged moieties. In some embodiments basic portion B does not include an amino acid. In embodiments of MTS molecules having features of the invention, a basic portion B may be a positively charged portion, having between about 5 and about 20 positive charges at physiological pH. In some embodiments, the amount of negative charge in portion A is approximately the same as the amount of positive charge in portion B. In some embodiments, the T-Y pair is attached to either end of B.

In some embodiments, A is an antibody. In some embodiments, A is an anti-ErbB antibody. In some embodiments, A is an anti-HER2 antibody. In some embodiments, the antibody is an anti-EGFR antibody. In some embodiments, A is an antibody selected from the group consisting of cetuximab, trastuzumab, and pertruzumab. In some embodiments, A is cetuximab. In some embodiments, A is trastuzumab. In some embodiments, A is pertruzumab.

A cargo moiety C (also referred to as a detectable moiety) and or compound T include, for example, a variety of detectable agents, including for example any detectable moiety for detection in an ex vivo assay, a contrast agent for diagnostic imaging, or a chemotherapeutic drug or radiation-sensitizer for therapy. B may be, for example, a peptide portion having between about 5 to about 20 basic amino acids, such as a series of basic amino acids (arginines are can be employed, as well as histidines, lysines or other basic amino acids). In some embodiments, X and/or Y is a cleavable linker that is cleavable under physiological conditions. A may be a peptide portion having between about 2 to about 20 about 2 to about 20 acidic amino acids, such as a series of acidic amino acids. In some embodiments of molecules having features of the invention, glutamates and aspartates are employed as acidic amino acids for peptide portion A.

A pre-targeting moiety P can include a variety of peptides for use in targeting the MTS molecules of the invention to a particular tissue, cell type and/or cell. A pre-targeting moiety P includes any moiety capable of binding a moiety on a target cell or in target cell location, including for example peptide moieties, antibodies, aptamers, chemical ligands, small molecule ligands, peptides, nucleotides/nucleic acids, peptide nucleic acids, locked nucleic acids and small molecule moieties as well as any derivatives thereof. In some embodiments, P includes any moiety capable of binding to cell and/or tissue involved in inflammation, diabetes, cardiovascular diseases, neurodegenerative disorders and cancer. In some embodiments, the receptor for which P is a legend can include any receptor differentially expressed on a neoplastic cell as compared to a non-neoplastic cell, including breast, prostate, liver, colon, lung, pancreas, stomach, brain, liver, kidney, bladder, blood or any other receptor known by those of skill in the art to be differentially expressed in cancer versus normal cells. In some embodiments, the receptor to which P is a ligand includes a receptor selected from but not limited to G-protein coupled receptors (GPCRs), androgen receptors (ARs), estrogen receptors (ERs), leptin receptors (LRs), growth hormone receptors (GHRs), transforming growth factor receptors (TGFs; including for example but not limited to TGF431, TGF432 and TGFβ3), epidermal growth factor receptors (EGFRs), HER2/neu receptors, breast cancer associated receptors (including for example but not limited to estrogen receptors), ErbB receptors, ErbB2 receptors, epidermal growth factor receptors (EGFRs), insulin like growth factor receptors (ILGFRs), HGF/Met receptors, tyrosine kinase receptors, pattern recognition receptors (PRRs), Toll-like receptors (TLRs) pathogen-associated molecular patterns (PAMP), killer activated and killer inhibitor receptors (KARs and KIRs), complement receptors, Fc receptors, B-cell receptors, T-cell receptors, cytokine receptors, RAGE, BTLA, protease activate receptors (PARs), nuclear receptors (including for example but not limited to PPARs), mineralocorticoid receptors, platelet ADP receptors, APJ receptor, muscarinic receptors (including for example but not limited to muscarinic acetylcholine receptor M2, M3 muscarinic receptor), glucorticoid receptors, adrenergic receptors, scavenger receptors, calcium sensing receptor (CaR), angiotension II receptor, bile acid receptors, corticosteroid receptors, Protease-activated receptors (PARs), interleukin receptors (including for example, but not limited to interleukin 1 receptors), AMPA receptors, insulin receptors, glucose receptors, cannabinoid receptors, chemokine receptors, N-methyl-D-aspartate (NDMA) receptors, adenosine receptors, peripheral benzodiazepine receptors, sigma-1 receptor, Trk receptors (including for example but not limited to TrkB receptor), nuclear hormone receptors, nicotinic receptors, nicotinic acetylcholine receptors (including for example but not limited to α4β2 and IgG receptors) and integrins. In some embodiments, P is a ligand for capable of binding to αvβ3 or αvβ5. In some embodiments, P is a ligand capable of binding to αvβ3. In some embodiments, P is a cyclic ligand. In some embodiments, P is cyclic RGD (cRGD).

The present inventors have made the surprising discovery that including an acidic portion A is also effective to inhibit or prevent the uptake into cells of molecules combining a portion B and a portion C and/or compound T. The present inventors have made the further discovery that cleavage of linker X and/or Y, allowing the separation of portion A from portion B is effective to allow the uptake of portions B and C into cells. Thus, delivery of cargo C can be controlled and enhanced by molecules having features of the invention.

For example, when peptide portion A contains about 5 to about 9 consecutive glutamates or aspartates, and X and/or Y is a flexible linker of about 2 to about 100, or about 6 to about 30 atoms in length, the normal ability of a peptide portion B (e.g., a sequence of nine consecutive arginine residues) to cause uptake into cells is blocked. Cleavage of linker X allows the separation of portion A from portion B and portion C and/or compound T, alleviating the veto by portion A. Thus, when separated from A, the normal ability of portion B to affect the uptake of cargo C into cells is regained. Such cellular uptake typically occurs near the location of the cleavage event. Thus, design of cleavable linker X and/or Y such that either is cleaved at or near a target cell is effective to direct uptake of cargo C and/or therapeutic moiety T into target cells. Extracellular cleavage of X and/or Y allows separation of A from the rest of the molecule to allow uptake into cells.

A MTS molecule having features of the invention may be of any length. In embodiments of MTS molecules having features of the invention, a MTS molecule may be about 7 to about 40 amino acids in length, not including the length of a linker X and/or Y, a cargo portion C and/or a moiety T. In other embodiments, particularly where multiple non-acidic (in portion A) or non-basic (in portion B) amino acids are included in one or both of portions A and B, portions A and B of a MTS molecule may together be about 50, or about 60, or about 70 amino acids in length. A cyclic portion of an MTS may include about 12 to about 60 amino acids, not including the length of a linker X and a cargo portion C. For example, a linear MTS molecule having features of the invention may have a basic portion B having between about 5 to about 20 basic amino acids (in some embodiments between about 9 to about 16 basic amino acids) and an acidic portion A having between about 2 to about 20 acidic amino acids (e.g., between about 5 to about 20, between about 5 to about 9 acidic amino acids). In some embodiments, a MTS molecule having features of the invention may have a basic portion B having between about 9 to about 16 basic amino acids and between about 5 to about 9 acidic amino acids.

Portions A and B may include either L-amino acids or D-amino acids. In embodiments of the invention, D-amino acids are employed for the A and B portions in order to minimize immunogenicity and nonspecific cleavage by background peptidases or proteases. Cellular uptake of oligo-D-arginine sequences is known to be as good or better than that of oligo-L-arginines. The generic structures of the molecules described above can be effective where A is at the amino terminus and where A is at the carboxy terminus, i.e. either orientation of the peptide bonds is permissible. However, in embodiments where X and/or Y is a peptide cleavable by a protease, it may be beneficial to join the C-terminus of X and/or Y to the N-terminus of B, so that the new amino terminus created by cleavage of X and/or Y contributes an additional positive charge that adds to the positive charges already present in B.

In some embodiments, a MTS molecule disclosed herein has the formula according to one of the following:

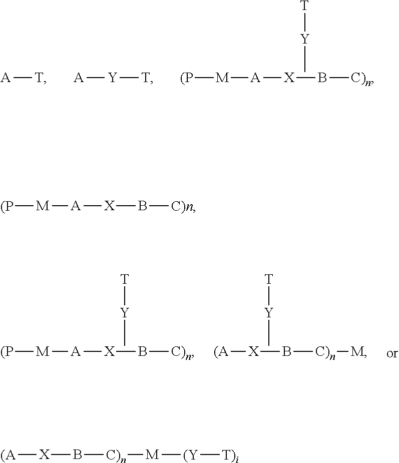

wherein C is a cargo moiety; T is a therapeutic moiety; A is a peptide with a sequence comprising 5 to 9 consecutive acidic amino acids, wherein the amino acids are selected from: aspartates and glutamates; B is a peptide with a sequence comprising 5 to 20 consecutive basic amino acids; and X and/or Y is a linker that is cleavable by thrombin. In some embodiments, the acid amino acids are consecutive. In some embodiments, the acid amino acids are not consecutive. In some embodiments of molecules having features of the invention, n and i are independently selected integers between 1 and 20. In some embodiments of molecules having features of the invention, n is an integer between 1 and 20. In some embodiments of molecules having features of the invention, n is an integer between 1 and 15. In some embodiments of molecules having features of the invention, n is an integer between 1 and 10. In some embodiments of molecules having features of the invention, n is an integer between 1 and 5. In some embodiments of molecules having features of the invention, n is an integer between 1 and 4. In some embodiments of molecules having features of the invention, n is an integer between 1 and 3. In some embodiments of molecules having features of the invention, n is an integer between 1 and 2. In some embodiments of molecules having features of the invention, n is 1. In some embodiments of molecules having features of the invention, n is an integer of 1, 2, 3, 4, 5, 6, 7, 8, 9, 10, 11, 12, 13, 14, 15, 16, 17, 18, 19, or 20. In some embodiments of molecules having features of the invention, i is an integer between 1 and 20. In some embodiments of molecules having features of the invention, i is an integer between 1 and 10. In some embodiments of molecules having features of the invention, i is an integer between 1 and 5. In some embodiments of molecules having features of the invention, i is an integer between 1 and 4. In some embodiments of molecules having features of the invention, i is an integer between 1 and 3. In some embodiments of molecules having features of the invention, i is an integer between 1 and 2. In some embodiments of molecules having features of the invention, i is 1. In some embodiments of molecules having features of the invention, i is an integer of 1, 2, 3, 4, 5, 6, 7, 8, 9, 10, 11, 12, 13, 14, 15, 16, 17, 18, 19, or 20. In some embodiments, the T-Y pair is attached to either end of B.

In some embodiments, A is an antibody. In some embodiments, A is an anti-ErbB antibody. In some embodiments, A is an anti-HER2 antibody. In some embodiments, the antibody is an anti-EGFR antibody. In some embodiments, A is an antibody selected from the group consisting of cetuximab, trastuzumab, and pertruzumab. In some embodiments, A is cetuximab. In some embodiments, A is trastuzumab. In some embodiments, A is pertruzumab.

In some embodiments, a MTS molecule disclosed herein has the formula according to one of the following:

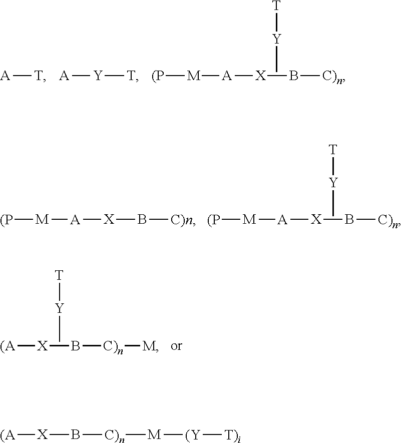

wherein C is a cargo moiety; T is a therapeutic moiety; A is a peptide with a sequence comprising 5 to 9 consecutive acidic amino acids, wherein the amino acids are selected from: aspartates and glutamates; B is a peptide with a sequence comprising 5 to 20 consecutive basic amino acids; X and/or Y is a linker that is cleavable by thrombin; M is a macromolecular carrier; and n is an integer between 1 and 20. In some embodiments of molecules having features of the invention, n and i are independently selected integers between 1 and 20. In some embodiments of molecules having features of the invention, n is an integer between 1 and 20. In some embodiments of molecules having features of the invention, n is an integer between 1 and 15. In some embodiments of molecules having features of the invention, n is an integer between 1 and 10. In some embodiments of molecules having features of the invention, n is an integer between 1 and 5. In some embodiments of molecules having features of the invention, n is an integer between 1 and 4. In some embodiments of molecules having features of the invention, n is an integer between 1 and 3. In some embodiments of molecules having features of the invention, n is an integer between 1 and 2. In some embodiments of molecules having features of the invention, n is 1. In some embodiments of molecules having features of the invention, n is an integer of 1, 2, 3, 4, 5, 6, 7, 8, 9, 10, 11, 12, 13, 14, 15, 16, 17, 18, 19, or 20. In some embodiments of molecules having features of the invention, i is an integer between 1 and 20. In some embodiments of molecules having features of the invention, i is an integer between 1 and 10. In some embodiments of molecules having features of the invention, i is an integer between 1 and 5. In some embodiments of molecules having features of the invention, i is an integer between 1 and 4. In some embodiments of molecules having features of the invention, i is an integer between 1 and 3. In some embodiments of molecules having features of the invention, i is an integer between 1 and 2. In some embodiments of molecules having features of the invention, i is 1. In some embodiments of molecules having features of the invention, i is an integer of 1, 2, 3, 4, 5, 6, 7, 8, 9, 10, 11, 12, 13, 14, 15, 16, 17, 18, 19, or 20.

In some embodiments of molecules having features of the invention, peptide portion A includes between about 2 to about 20, or between about 5 to about 20 acidic amino acids, and may be series of acidic amino acids (e.g., glutamates and aspartates or other acidic amino acids). In some embodiments, A has a sequence comprising 5 to 9 consecutive glutamates. In some embodiments, portion A comprises 8 consecutive glutamates (i.e., EEEEEEEE or eeeeeeee).

An acidic portion A may include amino acids that are not acidic. Acidic portion A may comprise other moieties, such as negatively charged moieties. In embodiments of a MTS molecule disclosed herein, an acidic portion A may be a negatively charged portion, in some embodiments having about 2 to about 20 negative charges at physiological pH that does not include an amino acid. In some embodiments, the amount of negative charge in portion A is approximately the same as the amount of positive charge in portion B.

In some embodiments, A is an antibody. In some embodiments, A is an anti-ErbB antibody. In some embodiments, A is an anti-HER2 antibody. In some embodiments, the antibody is an anti-EGFR antibody. In some embodiments, A is an antibody selected from the group consisting of cetuximab, trastuzumab, and pertruzumab. In some embodiments, A is cetuximab. In some embodiments, A is trastuzumab. In some embodiments, A is pertruzumab.

Portion A is either L-amino acids or D-amino acids. In embodiments of the invention, D-amino acids are can be employed in order to minimize immunogenicity and nonspecific cleavage by background peptidases or proteases. Cellular uptake of oligo-D-arginine sequences is known to be as good as or better than that of oligo-L-arginines.

It will be understood that portion A may include non-standard amino acids, such as, for example, hydroxylysine, desmosine, isodesmosine, or other non-standard amino acids. Portion A may include modified amino acids, including post-translationally modified amino acids such as, for example, methylated amino acids (e.g., methyl histidine, methylated forms of lysine, etc.), acetylated amino acids, amidated amino acids, formylated amino acids, hydroxylated amino acids, phosphorylated amino acids, or other modified amino acids. Portion A may also include peptide mimetic moieties, including portions linked by non-peptide bonds and amino acids linked by or to non-amino acid portions.

The generic structures according to one of the following:

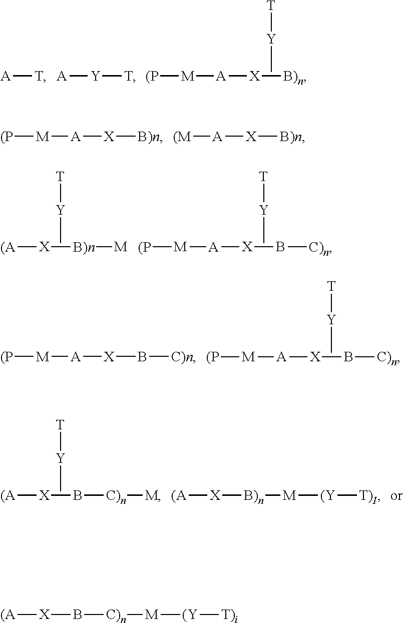

is effective where A is at the amino terminus or where A is at the carboxy terminus, i.e., either orientation of the peptide bonds is permissible. In some embodiments of molecules having features of the invention, n and i are independently selected integers between 1 and 20. In some embodiments of molecules having features of the invention, n is an integer between 1 and 20. In some embodiments of molecules having features of the invention, n is an integer between 1 and 15. In some embodiments of molecules having features of the invention, n is an integer between 1 and 10. In some embodiments of molecules having features of the invention, n is an integer between 1 and 5. In some embodiments of molecules having features of the invention, n is an integer between 1 and 4. In some embodiments of molecules having features of the invention, n is an integer between 1 and 3. In some embodiments of molecules having features of the invention, n is an integer between 1 and 2. In some embodiments of molecules having features of the invention, n is 1. In some embodiments of molecules having features of the invention, n is an integer of 1, 2, 3, 4, 5, 6, 7, 8, 9, 10, 11, 12, 13, 14, 15, 16, 17, 18, 19, or 20. In some embodiments of molecules having features of the invention, i is an integer between 1 and 20. In some embodiments of molecules having features of the invention, i is an integer between 1 and 10. In some embodiments of molecules having features of the invention, i is an integer between 1 and 5. In some embodiments of molecules having features of the invention, i is an integer between 1 and 4. In some embodiments of molecules having features of the invention, i is an integer between 1 and 3. In some embodiments of molecules having features of the invention, i is an integer between 1 and 2. In some embodiments of molecules having features of the invention, i is 1. In some embodiments of molecules having features of the invention, i is an integer of 1, 2, 3, 4, 5, 6, 7, 8, 9, 10, 11, 12, 13, 14, 15, 16, 17, 18, 19, or 20. In some embodiments, the T-Y pair is attached to either end of B.

In some embodiments, A is an antibody. In some embodiments, A is an anti-ErbB antibody. In some embodiments, A is an anti-HER2 antibody. In some embodiments, the antibody is an anti-EGFR antibody. In some embodiments, A is an antibody selected from the group consisting of cetuximab, trastuzumab, and pertruzumab. In some embodiments, A is cetuximab. In some embodiments, A is trastuzumab. In some embodiments, A is pertruzumab.

In some embodiments, a MTS molecule disclosed herein has the formula according to one of the following:

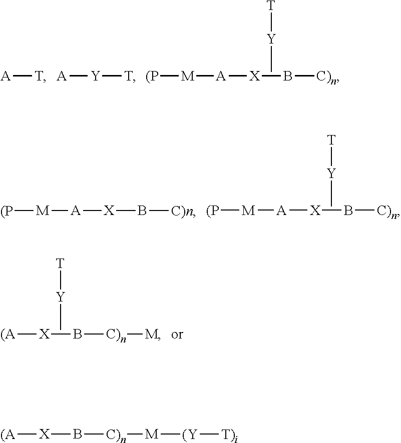

wherein C is a cargo moiety, A is a peptide with a sequence comprising 5 to 9 consecutive acidic amino acids, wherein the amino acids are selected from: aspartates and glutamates; B is a peptide with a sequence comprising 5 to 20 consecutive basic amino acids; and X and/or Y is a linker that is cleavable by thrombin. In some embodiments of molecules having features of the invention, n and i are independently selected integers between 1 and 20. In some embodiments of molecules having features of the invention, n is an integer between 1 and 20. In some embodiments of molecules having features of the invention, n is an integer between 1 and 15. In some embodiments of molecules having features of the invention, n is an integer between 1 and 10. In some embodiments of molecules having features of the invention, n is an integer between 1 and 5. In some embodiments of molecules having features of the invention, n is an integer between 1 and 4. In some embodiments of molecules having features of the invention, n is an integer between 1 and 3. In some embodiments of molecules having features of the invention, n is an integer between 1 and 2. In some embodiments of molecules having features of the invention, n is 1. In some embodiments of molecules having features of the invention, n is an integer of 1, 2, 3, 4, 5, 6, 7, 8, 9, 10, 11, 12, 13, 14, 15, 16, 17, 18, 19, or 20. In some embodiments of molecules having features of the invention, i is an integer between 1 and 20. In some embodiments of molecules having features of the invention, i is an integer between 1 and 10. In some embodiments of molecules having features of the invention, i is an integer between 1 and 5. In some embodiments of molecules having features of the invention, i is an integer between 1 and 4. In some embodiments of molecules having features of the invention, i is an integer between 1 and 3. In some embodiments of molecules having features of the invention, i is an integer between 1 and 2. In some embodiments of molecules having features of the invention, i is 1. In some embodiments of molecules having features of the invention, i is an integer of 1, 2, 3, 4, 5, 6, 7, 8, 9, 10, 11, 12, 13, 14, 15, 16, 17, 18, 19, or 20.

In some embodiments, A is an antibody. In some embodiments, A is an anti-ErbB antibody. In some embodiments, A is an anti-HER2 antibody. In some embodiments, the antibody is an anti-EGFR antibody. In some embodiments, A is an antibody selected from the group consisting of cetuximab, trastuzumab, and pertruzumab. In some embodiments, A is cetuximab. In some embodiments, A is trastuzumab. In some embodiments, A is pertruzumab.

In some embodiments, a MTS molecule disclosed herein has the formula according to one of the following:

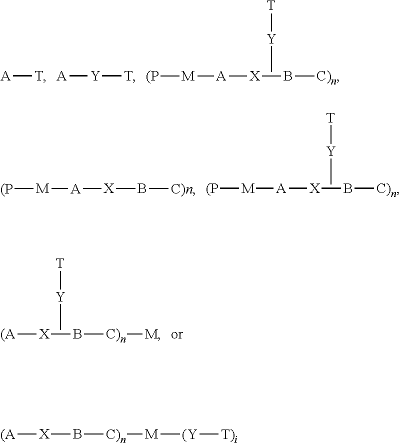

wherein C is a cargo moiety; A is a peptide with a sequence comprising 5 to 9 consecutive acidic amino acids, wherein the amino acids are selected from: aspartates and glutamates; B is a peptide with a sequence comprising 5 to 20 consecutive basic amino acids; X and/or Y is a linker that is cleavable by thrombin; M is a macromolecular carrier; and n is an integer between 1 and 20. In some embodiments of molecules having features of the invention, n and i are independently selected integers between 1 and 20. In some embodiments of molecules having features of the invention, n is an integer between 1 and 20. In some embodiments of molecules having features of the invention, n is an integer between 1 and 15. In some embodiments of molecules having features of the invention, n is an integer between 1 and 10. In some embodiments of molecules having features of the invention, n is an integer between 1 and 5. In some embodiments of molecules having features of the invention, n is an integer between 1 and 4. In some embodiments of molecules having features of the invention, n is an integer between 1 and 3. In some embodiments of molecules having features of the invention, n is an integer between 1 and 2. In some embodiments of molecules having features of the invention, n is 1. In some embodiments of molecules having features of the invention, n is an integer of 1, 2, 3, 4, 5, 6, 7, 8, 9, 10, 11, 12, 13, 14, 15, 16, 17, 18, 19, or 20. In some embodiments of molecules having features of the invention, i is an integer between 1 and 20. In some embodiments of molecules having features of the invention, i is an integer between 1 and 10. In some embodiments of molecules having features of the invention, i is an integer between 1 and 5. In some embodiments of molecules having features of the invention, i is an integer between 1 and 4. In some embodiments of molecules having features of the invention, i is an integer between 1 and 3. In some embodiments of molecules having features of the invention, i is an integer between 1 and 2. In some embodiments of molecules having features of the invention, i is 1. In some embodiments of molecules having features of the invention, i is an integer of 1, 2, 3, 4, 5, 6, 7, 8, 9, 10, 11, 12, 13, 14, 15, 16, 17, 18, 19, or 20. In some embodiments, the T-Y pair is attached to either end of B.

In some embodiments of molecules having features of the disclosure, peptide portion B includes between about 5 to about 20, or between about 9 to about 16 basic amino acids, and may be a series of basic amino acids (e.g., arginines, histidines, lysines, or other basic amino acids). In some embodiments, portion B comprises 9 consecutive arginines (i.e., RRRRRRRRR or rrrrrrrrr). In some embodiments, the basic amino acids are consecutive. In some embodiments, the basic amino acids are not consecutive.

A basic portion B may include amino acids that are not basic. Basic portion B may comprise other moieties, such as positively charged moieties. In embodiments, a basic portion B may be a positively charged portion, having between about 5 and about 20 positive charges at physiological pH that does not include an amino acid. In some embodiments, the amount of negative charge in portion A is approximately the same as the amount of positive charge in portion B.

Portion B is either L-amino acids or D-amino acids. In some embodiments of the invention, D-amino acids are employed in order to minimize immunogenicity and nonspecific cleavage by background peptidases or proteases. Cellular uptake of oligo-D-arginine sequences is known to be as good as or better than that of oligo-L-arginines.

It will be understood that portion B may include non-standard amino acids, such as, for example, hydroxylysine, desmosine, isodesmosine, or other non-standard amino acids. Portion B may include modified amino acids, including post-translationally modified amino acids such as, for example, methylated amino acids (e.g., methyl histidine, methylated forms of lysine, etc.), acetylated amino acids, amidated amino acids, formylated amino acids, hydroxylated amino acids, phosphorylated amino acids, or other modified amino acids. Portion B may also include peptide mimetic moieties, including portions linked by non-peptide bonds and amino acids linked by or to non-amino acid portions.

In some embodiments, A is an antibody. In some embodiments, A is an anti-ErbB antibody. In some embodiments, A is an anti-HER2 antibody. In some embodiments, the antibody is an anti-EGFR antibody. In some embodiments, A is an antibody selected from the group consisting of cetuximab, trastuzumab, and pertruzumab. In some embodiments, A is cetuximab. In some embodiments, A is trastuzumab. In some embodiments, A is pertruzumab.

In embodiments where X and/or Y is a peptide cleavable by a protease, it may be beneficial to join the C-terminus of X and/or Y to the N-terminus of B, so that the new amino terminus created by cleavage of X and/or Y contributes an additional positive charge that adds to the positive charges already present in B.

Cargo portion C may be attached to B in any location or orientation. A cargo portion C need not be located at an opposite end of portion B than a linker X and/or Y. Any location of attachment of C to B is acceptable as long as that attachment remains after X and/or Y is cleaved. For example, a cargo portion C may be attached to or near to an end of portion B with linker X and/or Y attached to an opposite end of portion B. A cargo portion C may also be attached to or near to an end of portion B with linker X and/or Y attached to or near to the same end of portion B. In some embodiments of the invention, a linker X and/or Y may link to a cargo portion C which is linked to a basic portion B where a MTS molecule having features of the invention comprising a cargo portion C linked to multiple basic portions B, each of which basic portions B are linked to a linker portion X and/or Y, and via the linker to an acidic portion A.

A linker X and/or Y may be designed for cleavage in the presence of particular conditions or in a particular environment. In some embodiments, a linker X and/or Y is cleavable under physiological conditions. Cleavage of such a linker X and/or Y may, for example, be enhanced or may be affected by particular pathological signals or a particular environment related to cells in which cargo delivery is desired. The design of a linker X and/or Y for cleavage by specific conditions, such as by a specific enzyme, allows the targeting of cellular uptake to a specific location where such conditions obtain. Thus, one important way that MTS molecules having features of the invention provide specific detection of specific proteases presence is by the design of the linker portion X and/or Y to be cleaved by the protease. Thus, another important way that MTS molecules having features of the invention provide specific detection of hydrogen peroxide presence is by the design of the linker portion X and/or Y to be cleaved by hydrogen peroxide. The linker portion X and/or Y can be designed to be cleaved only by specific proteases or to be selective for specific proteases. After cleavage of a linker X and/or Y, the portions B-C of the molecule are then a simple conjugate of B and C, in some instances retaining a relatively small, inert stub remaining from a residual portion of linker X and/or Y.

A linker portion X and/or Y may be cleavable by conditions found in the extracellular environment, such as acidic conditions which may be found near cancerous cells and tissues or a reducing environment, as may be found near hypoxic or ischemic cells and tissues; by proteases or other enzymes found on the surface of cells or released near cells having a condition to be treated, such as diseased, apoptotic or necrotic cells and tissues; or by other conditions or factors. An acid-labile linker may be, for example, a cis-aconitic acid linker. A linker portion X and/or Y may also be cleaved extracellularly in an ex vivo reaction. Other examples of pH-sensitive linkages include acetals, ketals, activated amides such as amides of 2,3-dimethylmaleamic acid, vinyl ether, other activated ethers and esters such as enol or silyl ethers or esters, imines, iminiums, enamines, carbamates, hydrazones, and other linkages. A linker X may be an amino acid or a peptide. A linker portion X and/or Y may also be cleaved by hydrogen peroxide found in the extracellular environment, which can occur near and around cells during inflammation, diabetes, cardiovascular diseases, neurodegenerative disorders and cancer. A peptide linker may be of any suitable length, such as, for example, about 3 to about 30, or about 6 to about 24 atoms in sequence (e.g., a linear peptide about 1 to 10 or about 2 to 8 amino acids long). A cleavable peptide linker may include an amino acid sequence recognized and cleaved by a protease, so that proteolytic action of the protease cleaves the linker X and/or Y. A cleavable peptide linker may include an amino acid sequence recognized and cleaved by a hydrogen peroxide, so that hydrogen peroxide action cleaves the linker X and/or Y. In some embodiments, cleavage of X and/or Y can allow for categorization of the tumor microenvironment.

In some embodiments, X and/or Y is a cleavable linker. In some embodiments, a linker X and/or Y is designed for cleavage in the presence of particular conditions or in a particular environment In some embodiments, a linker X and/or Y is cleavable under physiological conditions Cleavage of such a linker X and/or Y may, for example, be enhanced or may be affected by particular pathological signals or a particular environment related to cells in which cargo delivery is desired The design of a linker X and/or Y for cleavage by specific conditions, such as by a specific enzyme (e.g., thrombin), allows the targeting of cellular uptake to a specific location where such conditions obtain Thus, one important way that MTS molecules provide specific targeting of cellular uptake to desired cells, tissues, or regions is by the design of the linker portion X and/or Y to be cleaved by conditions near such targeted cells, tissues, or regions. After cleavage of a linker X and/or Y, the portions B-C, B-C with T, or B with T of the molecule are then a simple conjugate of the recited parts, in some instances retaining a relatively small, inert stub remaining from a residual portion of linker X and/or Y. In some embodiments, cleavage of X and/or Y can allow for categorization of the tumor microenvironment. In some embodiments, cleavage of X and/or Y can allow for categorization of the tumor and the proteases expressed by the tumor.

In some embodiments, X and/or Y is cleavable by a protease associated with a disease, including but not limited to inflammation, diabetes, cardiovascular diseases, neurodegenerative disorders and cancer. In some embodiments, X and or Y is cleavable by a matrix metalloproteinase (including but not limited to MMP-2, MMP-9, MMP-7 and MMP-14), a hydrogen peroxidase, cathepsins (including but not limited to cathepsin B and cathepsin K), serine proteases (including but not limited to neutrophil serine proteases), mast cell proteases, elastases, gelatinases (including but not limited to MMP-2/gelatinase A and MMP-9/92-kDa gelatinase B), collagenases (including but not limited to MMP-1, MMP-3, MMP-8, and MMP-13), stromelysins (including but not limited to MMP-3, -7, -10, -11, and -12), tissue inhibitors of metalloproteinases (TIMPs; including but not limited to TIMP-1 and TIMP-2) cysteine proteases, threonine proteases, aspartic proteases, thrombin, plasmin, PSA, trypsin, uPA, TOP, caspase (including but not limited to caspase-3), β-amyloid proteases, calpains, and presenilinases. For a description of proteases, see, for example, Choi et al., Theranostics, 2(2): 156-178 (2012); incorporated herein by reference in its entirety. In some embodiments, cleavage of X and/or Y can allow for categorization of the disease microenvironment. In some embodiments, cleavage of X and/or Y can allow for categorization of the tumor and the proteases expressed by the diseased cell(s).

In some embodiments, X and/or Y is DPRSFL, PPRSFL, Norleucine-TPRSF, PLGC(Me)AG, 6-aminohexanoyl, 5-amino-3-oxapentanoyl, Val-Cit-(p-amido)benzyloxycarbonyl (Val-Cit-PAB), benzyloxycarbonyl-valine-citrulline (Z-val-cit), or a combination thereof. In some embodiments, X is DPRSFL, PPRSFL, Norleucine-TPRSF, PLGC(Me)AG, SSKLQ, GPLGIAGQ, Glu-Pro-Cit-Gly-Hof-Tyr-Leu, PVGLIG, D-Ala-Phe-Lys, or a combination thereof. In some embodiments, X and/or Y is a p-amido-benzyl ether. In some embodiments, X is 6-aminohexanoyl, 5-amino-3-oxapentanoyl, or a combination thereof. In some embodiments, Y is Val-Cit-(p-amido)benzyloxycarbonyl (Val-Cit-PAB).

In some embodiments, X and/or Y is cleaved by thrombin. In some embodiments, X and/or Y is substantially specific for thrombin, MMPs or elastases. In some embodiments, X and/or Y is cleaved by or is substantially specific for MMPs (PLGLAG and PLGC(met)AG, elastases (RLQLK(acetyl)L, plasmin and/or thrombin. In some embodiments, X and/or Y is DPRSFL, PPRSFL, Norleucine-TPRSF, or PLGC(Me)AG. In some embodiments, X and/or Y is 6-aminohexanoyl, 5-amino-3-oxapentanoyl, or a combination thereof. In some embodiments, X and/or Y is a p-amido-benzyl ether, such as for example Val-Cit-(p-amido)benzyloxycarbonyl (Val-Cit-PAB). In some embodiments, X is DPRSFL, PPRSFL, Norleucine-TPRSF, or PLGC(Me)AG. In some embodiments, X is 6-aminohexanoyl, 5-amino-3-oxapentanoyl, or a combination thereof. In some embodiments, Y is Val-Cit-(p-amido)benzyloxycarbonyl (Val-Cit-PAB). In some embodiments, the MMP 2,9 cleavable or substantially specific sequence is PLGLAG and/or PLGC(met)AG. In some embodiments, the MMP cleavable or substantially specific sequences could include but are not limited to RS-(Cit)-G-(homoF)-YLY, PLGLEEA, CRPAHLRDSG, SLAYYTA, NISDLTAG, PPSSLRVT, SGESLSNLTA, RIGFLR elastase cleavable or substantially specific sequence is RLQLK(acetyl)L. In some embodiments, the plasmin cleavable or substantially specific sequence is RLQLKL. Thrombin selective substrates include DPRSFL, PPRSFL, and Norleucine-TPRSFL. In some embodiments, the chymase cleavable or substantially specific sequence GVAY|SGA. Urokinase-type plasminogen activator (uPA) and tissue plasminogen activator (tPA) cleavable or substantially specific sequence is YGRAAA. In some embodiments, the uPA cleavable or substantially specific sequence is YGPRNR. In some embodiments, X and Y are different cleavable linkers such that specific properties of the linkers are employed for delivery of cargo and therapeutic compounds to particular cells and/or cellular environments. In some embodiments, linker X and/or Y can be any combination of linkers described herein.

In some embodiments, X and/or Y is cleaved by a peroxide, including but not limited to hydrogen peroxide. Examples of X linkers cleaved by hydrogen peroxide include but are not limited to ACPP1 and/or ACPP2. The representative structure for ACPP 1 is:

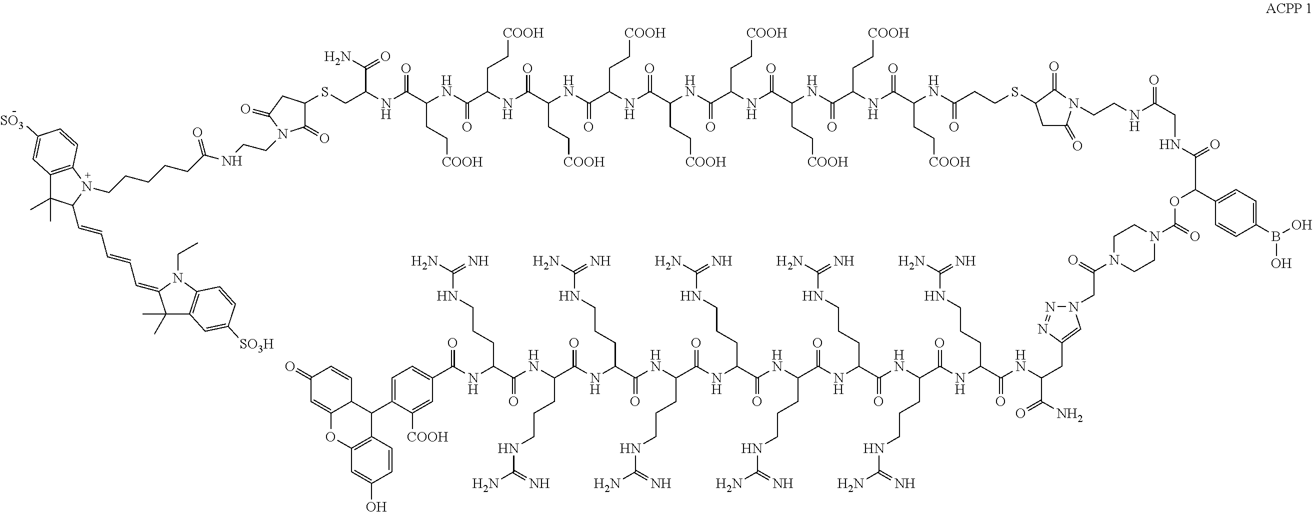

The representative structure for ACPP2 is:

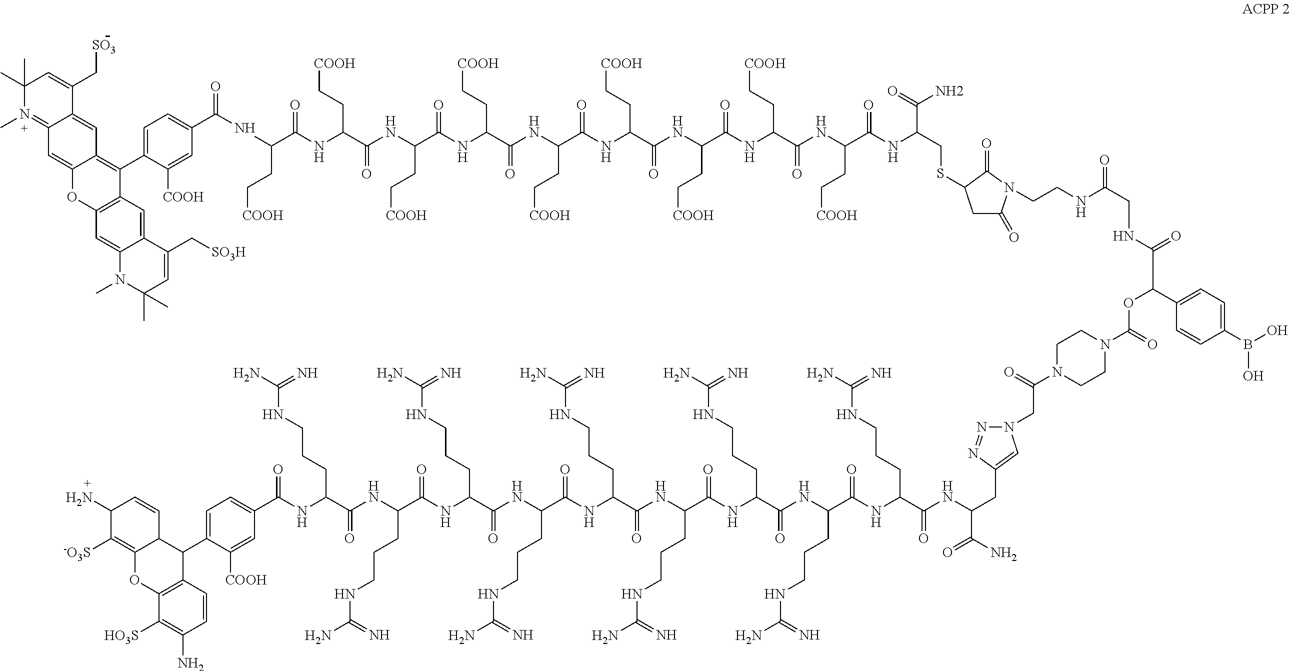

Other related structures cleavable by hydrogen peroxide are also contemplated by the present invention.

In some embodiments, a linker consisting of one or more amino acids is used to join peptide sequence A (i.e., the sequence designed to prevent uptake into cells) and peptide sequence B (i.e., the TS). Generally the peptide linker will have no specific biological activity other than to join the molecules or to preserve some minimum distance or other spatial relationship between them. However, the constituent amino acids of the linker may be selected to influence some property of the molecule such as the folding, net charge, or hydrophobicity.

In some embodiments, the linker is flexible. In some embodiments, the linker is rigid. In some embodiments, both linkers are flexible. In some embodiments, both linkers are rigid. In some embodiments, one linker is rigid and one linker is flexible.

In some embodiments, one or both linkers comprise a linear structure. In some embodiments, one or both linkers comprise a non-linear structure. In some embodiments, one or both linkers comprise a branched structure. In some embodiments, one or both linkers comprise a cyclic structure. In some embodiments, the linkers comprise the same structure. In some embodiments, the linkers comprise different structures.

In some embodiments, X and/or Y is about 5 to about 30 atoms in length. In some embodiments, X and/or Y is about 6 atoms in length. In some embodiments, X and/or Y is about 8 atoms in length. In some embodiments, X and/or Y is about 10 atoms in length. In some embodiments, X and/or Y is about 12 atoms in length. In some embodiments, X and/or Y is about 14 atoms in length. In some embodiments, X and/or Y is about 16 atoms in length. In some embodiments, X and/or Y is about 18 atoms in length. In some embodiments, X and/or Y is about 20 atoms in length. In some embodiments, X and/or Y is about 25 atoms in length. In some embodiments, X and/or Y is about 30 atoms in length.

In some embodiments, X and/or Y is cleaved by thrombin. In some embodiments, the linker is substantially specific for thrombin.

In some embodiments, the linker X and/or Y has a formula selected from: DPRSFL, PPRSFL, or Norleucine-TPRSF. In some embodiments, linker is DPRSFL, PPRSFL, or Norleucine-TPRSF.

In some embodiments, the linker X and/or Y binds peptide portion A (i.e., the peptide sequence which prevents cellular uptake) to peptide portion B (i.e., the MTS sequence) by a covalent linkage. In some embodiments, the covalent linkage comprises an ether bond, thioether bond, amine bond, amide bond, carbon-carbon bond, carbon-nitrogen bond, carbon-oxygen bond, or carbon-sulfur bond.

In some embodiments, X and/or Y comprises a peptide linkage. The peptide linkage comprises L-amino acids and/or D-amino acids. In embodiments of the invention, D-amino acids are employed in order to minimize immunogenicity and nonspecific cleavage by background peptidases or proteases. Cellular uptake of oligo-D-arginine sequences is known to be as good as or better than that of oligo-L-arginines.

It will be understood that a linker disclosed herein may include non-standard amino acids, such as, for example, hydroxylysine, desmosine, isodesmosine, or other non-standard amino acids. A linker disclosed herein may include modified amino acids, including post-translationally modified amino acids such as, for example, methylated amino acids (e.g., methyl histidine, methylated forms of lysine, etc.), acetylated amino acids, amidated amino acids, formylated amino acids, hydroxylated amino acids, phosphorylated amino acids, or other modified amino acids. A linker disclosed herein may also include peptide mimetic moieties, including portions linked by non-peptide bonds and amino acids linked by or to non-amino acid portions.

In some embodiments, a MTS molecule disclosed herein comprises a single type of linker. Use of a single mechanism to mediate uptake of both imaging and therapeutic cargoes is particularly valuable, because imaging with noninjurous tracer quantities can be used to test whether a subsequent therapeutic dose is likely to concentrate correctly in the target tissue.

In some embodiments, a MTS molecule disclosed herein comprises different types of linkers. Use of multiple mechanisms to mediate uptake of both imaging and therapeutic cargoes is particularly valuable, because imaging with noninjurous tracer quantities can be used to test whether a subsequent therapeutic dose is likely to concentrate correctly in the target tissue.

In some embodiments, a MTS molecule disclosed herein comprises a plurality of linkers. Where a MTS molecule disclosed herein includes multiple linkages X and/or Y, separation of portion A from the other portions of the molecule requires cleavage of all linkages X and/or Y. Cleavage of multiple linkers X and/or Y may be simultaneous or sequential Multiple linkages X and/or Y may include linkages X having different specificities, linkages Y have different specificities and/or linkages X and/or Y having different specificities so that separation of portion A from the other portions of the molecule requires that more than one condition or environment (“extracellular signals”) be encountered by the molecule. Cleavage of multiple linkers X and/or Y thus serves as a detector of combinations of such extracellular signals. For example, a MTS molecule may include two linker portions Xa and Xb connecting basic portion B with acidic portion A. Both linkers Xa and Xb must be cleaved before acidic portion A is separated from basic portion B allowing entry of portion B and cargo moiety C or therapeutic moiety T (if any) to enter a cell. For example, a MTS molecule may include two linker portions Ya and Yb connecting basic portion B with acidic portion A. Both linkers Ya and Yb must be cleaved before acidic portion A is separated from basic portion B allowing entry of portion B and cargo moiety C or therapeutic moiety T (if any) to enter a cell. For example, a MTS molecule may include four linker portions Xa, Xb, Ya and Yb connecting basic portion B with acidic portion A. All four linkers Xa, Xb, Ya and Yb must be cleaved before acidic portion A is separated from basic portion B allowing entry of portion B and cargo moiety C or therapeutic moiety T (if any) to enter a cell. It will be understood that a linker region may link to either a basic portion B or a cargo moiety C or therapeutic moiety T independently of another linker that may be present, and that, where desired, more than two linker regions X may be included.

Combinations of two or more linkers X and/or Y may be used to further modulate the detection of multiple proteases with a single MTS molecule, as well as targeting and delivery of molecules to desired cells, tissue or regions. Combinations of extracellular signals are used to widen or narrow the specificity of the cleavage of linkers X and/or Y if desired. Where multiple linkers X and/or Y are linked in parallel, the specificity of cleavage is narrowed, since each linker X must be cleaved before portion. A may separate from the remainder of the molecule. Where multiple linkers X and/or Y are linked in series, the specificity of cleavage is broadened, since cleavage on any one linker X and/or Y allows separation of portion A from the remainder of the molecule. For example, in order to detect either a protease OR hypoxia (i.e., to cleave X and/or Y in the presence of either protease or hypoxia), a linker X and/or Y is designed to place the protease-sensitive and reduction-sensitive sites in tandem, so that cleavage of either would suffice to allow separation of the acidic portion. Alternatively, in order to detect the presence of both a protease and hypoxia (i.e., to cleave X and/or Y in the presence of both protease and hypoxia but not in the presence of only one alone), a linker X and/or Y is designed to place the protease sensitive site between at least one pair of cysteines that are disulfide-bonded to each other In that case, both protease cleavage and disulfide reduction are required in order to allow separation of portion A.

One important class of signals is the hydrolytic activity of matrix metalloproteinases (MMPs), which are very important in the invasive migration of metastatic tumor cells. MMPs are also believed to play major roles in inflammation and stroke. MMPs are reviewed in Visse et al., Circ. Res. 92:827-839 (2003). MMPs may be used to cleave a linker X and/or Y and so to allow separation of acidic portion A from portions B and C, allowing cellular uptake of cargo C and/or therapeutic compound T (e.g., radiosensitizing agent T) so that cellular uptake of C and/or T is triggered by action of MMPs. Such uptake is typically in the vicinity of the MMPs that trigger cleavage of X and/or Y. Thus, uptake of molecules having features of the invention are able to direct cellular uptake of cargo C and or therapeutic compound T (e.g., radiosensitizing agent T) to specific cells, tissues, or regions having active MMPs in the extracellular environment.

For example, a linker X and/or Y that includes the amino-acid sequence PLGLAG (SEQ ID NO: 1) may be cleaved by the metalloproteinase enzyme MMP-2 (a major MMP in cancer and inflammation). Cleavage of such a linker X and/or Y occurs between the central G and L residues, causing cell uptake to increase by 10 to 20-fold. A great deal is known about the substrate preferences of different MMPs, so that linkers X and/or Y may be designed that are able to bias X and/or Y to be preferentially sensitive to particular subclasses of MMPs, or to individual members of the large MMP family of proteinases. For example, in some embodiments, linkers X and/or Y designed to be cleaved by membrane-anchored MMPs are particularly employed because their activity remains localized to the outer surface of the expressing cell. In alternative embodiments, linkers X and/or Y designed to be cleaved by a soluble secreted MMP are employed where diffusion of cargo C and/or therapeutic compound T (e.g., radiosensitizing agent T) away from the exact location of cleavage may be desired, thereby increasing the spatial distribution of the cargo C and/or therapeutic compound T (e.g., radiosensitizing agent T). Other linkers X and/or Y cleavable by other MMPs are discussed throughout the disclosure.

Hypoxia is an important pathological signal. For example, hypoxia is thought to cause cancer cells to become more resistant to radiation and chemotherapy, and also to initiate angiogenesis. A linker X and/or Y suitable for cleavage in or near tissues suffering from hypoxia enables targeting of portion B and C and/or T to cancer cells and cancerous tissues, infarct regions, and other hypoxic regions. For example, a linker X and/or Y that includes a disulfide bond is preferentially cleaved in hypoxic regions and so targets cargo delivery to cells in such a region. In a hypoxic environment in the presence of, for example, leaky or necrotic cells, free thiols and other reducing agents become available extracellularly, while the 02 that normally keeps the extracellular environment oxidizing is by definition depleted. This shift in the redox balance should promote reduction and cleavage of a disulfide bond within a linker X and/or Y. In addition to disulfide linkages which take advantage of thiol-disulfide equilibria, linkages including quinones that fall apart when reduced to hydroquinones may be used in a linker X and/or Y designed to be cleaved in a hypoxic environment.

Necrosis often leads to release of enzymes or other cell contents that may be used to trigger cleavage of a linker X and/or Y. A linker X and/or Y designed for cleavage in regions of necrosis in the absence of hypoxia, for example, may be one that is cleaved by calpains or other proteases that may be released from necrotic cells. Such cleavage of linkers X by calpains would release the connected portions B-C and/or compound T (e.g., radiosensitizing agent T) from portion A allowing cargo C and/or therapeutic compound T (e.g., radiosensitizing agent T) to be taken up by diseased cells and by neighboring cells that had not yet become fully leaky.

Acidosis is also commonly observed in sites of damaged or hypoxic tissue, due to the Warburg shift from oxidative phosphorylation to anaerobic glycolysis and lactic acid production. Such local acidity could be sensed either by making an acid-labile linker X and/or Y (e.g., by including in X and/or Y an acetal or vinyl ether linkage). Alternatively, or in addition, acidosis may be used as a trigger of cargo C and/or therapeutic compound T (e.g., radiosensitizing agent T) uptake by replacing some of the arginines within B by histidines, which only become cationic below pH 7.

Molecules having features of the invention are suitable for carrying different cargoes C and/or compounds T, including different types of cargoes C and/or compounds T and different species of the same types of cargo C and/or compound T (e.g., radiosensitizing agent T), for uptake into cells. For example, different types of cargo include marker cargoes (e.g., fluorescent or radioactive label moieties) and different types of compounds T include therapeutic cargoes (e.g., chemotherapeutic molecules such as methotrexate or doxorubicin), or other cargoes C and/or compounds T. Where destruction of aberrant or diseased cells is therapeutically required, a cargo C and/or a compound T (e.g., radiosensitizing agent T) include a therapeutic cargo such as a “cytotoxic agent,” i.e. a substance that inhibits or prevents the function of cells and/or causes destruction of cells. In some embodiments, a single molecule having features of the invention includes more than one cargo portion C and/or compound T (e.g., radiosensitizing agent T) so that a basic portion B may be linked to multiple cargoes C and/or compounds T. Such multiple cargoes C and/or compounds T include marker cargoes, therapeutic cargoes, or other cargoes. Multiple cargoes C and compounds T may allow, for example, delivery of both a radioactive marker and an ultrasound or contrast agent, allowing imaging by different modalities. Alternatively, for example, delivery of a radioactive cargo C along with an anti-cancer agent compound T (e.g., radiosensitizing agent T), providing enhanced anticancer activity, or delivery of a radioactive cargo with a fluorescent cargo, allowing multiple means of localizing and identifying cells which have taken up cargo C and/or compound T (e.g., radiosensitizing agent T).

Delivery of cargo C and/or compound T (e.g., radiosensitizing agent T) such as a fluorescent molecule may be used to visualize cells having a certain condition or cells in a region exhibiting a particular condition. For example, thrombosis (clot formation) may be visualized by designing a linker X and/or Y to be cleaved by any of the many proteases in the blood clot formation cascade for delivery of a cargo including a fluorescent or other marker to the region. Similarly, complement activation may be visualized by designing a linker X and/or Y to be cleaved by any one or more of the proteases in the complement activation cascades for delivery of a fluorescent or other marker to the region. Thus, fluorescent molecules are one example of a marker that may be delivered to target cells and regions upon release of a portion A upon cleavage of a linker X and/or Y.

Delivery of compound T (e.g., radiosensitizing agent T) such as a therapeutic compound (also referred to herein as a “warhead”) may be used to treat cells having a certain condition or cells in a region exhibiting a particular condition. For example, neoplasia cells may be targeted by designing a linker X and/or Y to be cleaved by any of the many proteases produced by tumor cells, including for example MMPs. Thus, therapeutic compounds for the treatment of neoplasia are one example of compounds that may be delivered to target cells and regions upon release of a portion A and/or B upon cleavage of a linker X and/or Y. In some embodiments, a portion A (such as an antibody) delivers the compound T (e.g., radiosensitizing agent T) to tumor. In some embodiments, a portion A (such as an antibody) delivers the compound T (e.g., radiosensitizing agent T) to tumor and the tumor is sensitized to the radiosensitizing agent T.

A molecule having features of the invention may include one or more linkers X and/or Y so that an acidic portion A may be linked to portions B and C by one or more linkages. Such linkages connecting to portion A may be to portion B, to portion C, or to both portions B and C. Where a molecule having features of the invention includes multiple linkages X and/or Y, separation of portion A from the other portions of the molecule requires cleavage of all linkages X and/or Y. Cleavage of multiple linkers X and/or Y may be simultaneous or sequential. Multiple linkages X and/or Y may include linkages X and/or Y having different specificities, so that separation of portion A from the other portions of the molecule requires that more than one condition or environment (“extracellular signals”) be encountered by the molecule. Cleavage of multiple linkers X and/or Y thus serves as a detector of combinations of such extracellular signals. In some embodiments, MTS molecule having includes two linker portions Xa and Xb connecting basic portion B with acidic portion A. In some embodiments, a cyclic MTS molecule includes two linker regions Xa and Xb connecting basic portion B with acidic portion A. In some embodiments, both linkers Xa and Xb must be cleaved before acidic portion A is separated from basic portion B allowing entry of portion B and cargo portion C (if any) to enter a cell. In some embodiments, MTS molecule having includes two linker portions Ya and Yb connecting basic portion B with acidic portion A. In some embodiments, a cyclic MTS molecule includes two linker regions Ya and Yb connecting basic portion B with acidic portion A. In some embodiments, both linkers Ya and Yb must be cleaved before acidic portion A is separated from basic portion B allowing entry of portion B and cargo portion C (if any) to enter a cell. In some embodiments, MTS molecule having includes two linker portions X and Y connecting basic portion B with acidic portion A. In some embodiments, a cyclic MTS molecule includes two linker regions X and Y connecting basic portion B with acidic portion A. In some embodiments, both linkers X and Y must be cleaved before acidic portion A is separated from basic portion B allowing entry of portion B and cargo portion C (if any) to enter a cell. In some embodiments, MTS molecule having includes four linker portions Xa, Xb, Ya and Yb connecting basic portion B with acidic portion A. In some embodiments, a cyclic MTS molecule includes four linker regions Xa, Xb, Ya and Yb connecting basic portion B with acidic portion A. In some embodiments, linkers Xa, Xb, Ya and Yb must be cleaved before acidic portion A is separated from basic portion B allowing entry of portion B and cargo portion C (if any) to enter a cell. In some embodiments, MTS molecule having includes more than two portions X and X connecting basic portion B with acidic portion A. In some embodiments, a cyclic MTS molecule includes two linker regions X and Y connecting basic portion B with acidic portion A. In some embodiments, both linkers X and Y must be cleaved before acidic portion A is separated from basic portion B allowing entry of portion B and cargo portion C (if any) to enter a cell. It will be understood that a linker region may link to either a basic portion B or a cargo portion C and/or compound T (e.g., radiosensitizing agent T) independently of another linker that may be present, and that, where desired, more than two linker regions X and/or Y may be included.

Combinations of two or more linkers X and/or Y may be used to further modulate the targeting and delivery of molecules to desired cells, tissue or regions. Boolean combinations of extracellular signals can be detected to widen or narrow the specificity of the cleavage of linkers X and/or Y if desired. Where multiple linkers X and/or Y are linked in parallel, the specificity of cleavage is narrowed, since each linker X and/or Y must be cleaved before portion A may separate from the remainder of the molecule. Where multiple linkers X and/or Y are linked in series, the specificity of cleavage is broadened, since cleavage on any one linker X allows separation of portion A from the remainder of the molecule. For example, in order to detect either a protease OR hypoxia (i.e., to cleave X and/or Yin the presence of either protease or hypoxia), a linker X and/or Y is designed to place the protease-sensitive and reduction-sensitive sites in tandem, so that cleavage of either would suffice to allow separation of the acidic portion A. Alternatively, in order to detect the presence of both a protease AND hypoxia (i.e., to cleave X and/or Y in the presence of both protease and hypoxia but not in the presence of only one alone), a linker X and/or Y is designed to place the protease sensitive site between at least one pair of cysteines that are disulfide-bonded to each other. In that case, both protease cleavage and disulfide reduction are required in order to allow separation of portion A.

D amino acids may be used in MTS molecules having features of the invention. For example, some or all of the peptides of portions A and B may be D-amino acids in some embodiments of the invention. In an embodiment of the invention suitable for delivering a detectable marker to a target cell, a MTS having features of the invention includes a contrast agent as cargo C and/or compound T (e.g., radiosensitizing agent T) attached to a basic portion B comprising 8 to 10 D-arginines. Acidic portion A may include D-amino acids as well. Similarly, a drug may be delivered to a cell by such molecules having a basic portion B including 8 to 10 D-arginines and an acidic portion A including acidic D-amino acids.

It will be understood that a MTS molecule having features of the invention may include non-standard amino acids, such as, for example, hydroxylysine, desmosine, isodesmosine, or other non-standard amino acids. A MTS molecule having features of the invention may include modified amino acids, including post-translationally modified amino acids such as, for example, methylated amino acids (e.g., methyl histidine, methylated forms of lysine, etc.), acetylated amino acids, amidated amino acids, formylated amino acids, hydroxylated amino acids, phosphorylated amino acids, or other modified amino acids. A MTS molecule having features of the invention may also include peptide mimetic moieties, including portions linked by non-peptide bonds and amino acids linked by or to non-amino acid portions. For example, a MTS molecule having features of the invention may include peptoids, carbamates, vinyl polymers, or other molecules having non-peptide linkages but having an acidic portion cleavably linked to a basic portion having a cargo moiety.

The linker portion X and/or Y may be designed so that it is cleaved, for example, by proteolytic enzymes or reducing environment, as may be found near cancerous cells, or a hydrogen peroxide environment found near inflammatory diseases, neurodegenerative diseases, cardiovascular diseases, diabetes and cancer (neoplasia). Such an environment, or such enzymes or hydrogen peroxide, are typically not found near normal cells. In some embodiments, a cleavable linker X and/or Y is designed to be cleaved near diseased cells, including cancerous cells. In some embodiments, the cleavable linker is not cleaved near normal tissue. A capable of vetoing cellular uptake of a portion B, and of a portion B-C, and/or B-T blocking the entry of cargo and/or compound T (e.g., radiosensitizing agent T) into normal tissue.

In some embodiments, the linker portion X and/or Y may be cleaved, for example, by proteolytic enzymes, reducing environment or reactive oxygen species containing found near diseased cells, such as inflammatory diseased cells, neurodegenerative diseased cells, cardiovascular diseased cells, diabetic cells and cancerous cells to deliver a marker or a drug to cancerous cells. In some embodiments, a MTS molecule with a cleavable linker X and/or Y that is cleaved by proteolytic enzymes, by the reducing environment or by the reactive oxygen species containing environment near diseased cells is able to facilitate cargo entry into diseased tissue. Thus, the selective cleavage of the linker X and/or Y and the resulting separation of cargo C and/or compound T (e.g., radiosensitizing agent T) and basic portion B from acidic portion A allows the targeted uptake of cargo C and/or compound T (e.g., radiosensitizing agent T) into cells having selected features (e.g., enzymes), or located near to, a particular environment. Thus, molecules having features of the invention are able to selectively deliver cargo to target cells without doing so to normal or otherwise non-targeted cells.

In embodiments, a MTS molecule disclosed herein is a linear molecule. In embodiments, a MTS molecule disclosed herein is a cyclic molecule, as schematically illustrated in FIG. 1B of WO 2011/008996; incorporated herein by reference in its entirety. In embodiments, a MTS molecule disclosed herein comprises a cyclic portion and a linear portion.

A MTS disclosed herein may be of any length. In some embodiments, a MTS molecule disclosed herein is about 7 to about 40 amino acids in length, not including the length of a linker X and/or Y, a cargo moiety C and/or a compound T (e.g., radiosensitizing agent T). In other embodiments, particularly where multiple non-acidic (in portion A) or non-basic (in portion B) amino acids are included in one or both of portions A and B, portions A and B of a MTS molecule disclosed herein may together be about 50, or about 60, or about 70 amino acids in length. A cyclic portion of a MTS molecule disclosed herein may include about 12 to about 60 amino acids, not including the length of a linker X and/or Y, a cargo moiety C, and/or a compound T (e.g., radiosensitizing agent T). For example, a linear MTS molecule disclosed herein may have a basic portion B having between about 5 to about 20 basic amino acids (between about 9 to about 16 basic amino acids) and an acidic portion A having between about 2 to about 20 acidic amino acids (e.g., between about 5 to about 20, between about 5 to about 9 acidic amino acids). In some particular embodiments, a MTS molecule disclosed herein may have a basic portion B having between about 9 to about 16 basic amino acids and between about 5 to about 9 acidic amino acids. In some embodiments, A is consecutive glutamates (i.e., EEEEEEEE, E9, eeeeeeee, or e9), B is nine consecutive arginines (i.e., RRRRRRRRR, R9, rrrrrrrrr, or r9).

In some embodiments, the MTS is selected from: Suc-e9-XDPRSFL-r9-c(Cy5)-CONH2; Suc-e9-ODPRSFL-r9-c(Cy5)-CONH2; Suc-e9-Xdprsfl-r9-c(Cy5)-CONH2; cRGD-ACPP-MMAE; and (D-arg)9-Cy5 CPP-c(RGDfK)-(D-glu)9.

A MTS molecule disclosed herein may be of any length. In some embodiments, a MTS molecule disclosed herein is about 7 to about 40 amino acids in length, not including the length of a linker X and/or Y, a cargo moiety C and/or compound T (e.g., radiosensitizing agent T). In other embodiments, particularly where multiple non-acidic (in portion A) or non-basic (in portion B) amino acids are included in one or both of portions A and B, portions A and B of a MTS molecule disclosed herein may together be about 50, or about 60, or about 70 amino acids in length. A cyclic portion of a MTS molecule disclosed herein may include about 12 to about 60 amino acids, not including the length of a linker X and/or Y, a cargo moiety and/or compound T (e.g., radiosensitizing agent T).

For example, a linear MTS molecule disclosed herein may have a basic portion B having between about 5 to about 20 basic amino acids (in some embodiments between about 9 to about 16 basic amino acids) and an acidic portion A having between about 2 to about 20 acidic amino acids (e.g., between about 5 to about 20, between about 5 to about 9 acidic amino acids). In some embodiments, a MTS molecule disclosed herein may have a basic portion B having between about 9 to about 16 basic amino acids and between about 5 to about 9 acidic amino acids. In some embodiments, A is 9 consecutive glutamates (i.e., EEEEEEEE, E9, eeeeeeee, or e9), B is nine consecutive arginines (i.e., RRRRRRRRR, R9, rrrrrrrrr, or r9), and X and/or Y is PLGLAG. In some embodiments, A is 9 consecutive glutamates (i.e., EEEEEEEE, E9, eeeeeeee, or e9), B is nine consecutive arginines (i.e., RRRRRRRRR, R9, rrrrrrrrr, or r9), and X and/or Y is DPRSFL, PPRSFL, Norleucine-TPRSF, PLGC(Me)AG, 6-aminohexanoyl, 5-amino-3-oxapentanoyl, Val-Cit-(p-amido)benzyloxycarbonyl (Val-Cit-PAB) or a combination thereof. In some embodiments, A is 9 consecutive glutamates (i.e., EEEEEEEE, E9, eeeeeeee, or e9), B is nine consecutive arginines (i.e., RRRRRRRRR, R9, rrrrrrrrr, or r9), and X is PLGLAG, DPRSFL, PPRSFL, Norleucine-TPRSF, PLGC(Me)AG, or a combination thereof. In some embodiments, A is 9 consecutive glutamates (i.e., EEEEEEEE, E9, eeeeeeee, or e9), B is nine consecutive arginines (i.e., RRRRRRRRR, R9, rrrrrrrrr, or r9), and X is 6-aminohexanoyl, 5-amino-3-oxapentanoyl, or a combination thereof. In some embodiments, A is 9 consecutive glutamates (i.e., EEEEEEEE, E9, eeeeeeee, or e9), B is nine consecutive arginines (i.e., RRRRRRRRR, R9, rrrrrrrrr, or r9), and Y is Val-Cit-(p-amido)benzyloxycarbonyl (Val-Cit-PAB).

In some embodiments, the MTS molecule has a formula given below. It should be noted that in some instances the peptide sequence is given by the amino acid symbol and a number indicating the number of amino acids (for example, R9 translates to RRRRRRRRR or nine consecutive L-arginines; and r9 translates to nine consecutive D-arginines or rrrrrrrrr and E9 translates to EEEEEEEEE or nine consecutive L-glutamates; and e9 translates to nine consecutive D-glutamates or eeeeeeeee):
cRGD-MMP-MMAE (Ligand=Cyclic(RGDfC); Substrate=o-PLGC(Me)AG-o; o=5-amino-3-oxopentanoyl)
cRAD-PEG6-MMAE (Ligand=Cyclic(RADfC); Substrate=PEG6-o; o=5-amino-3-oxopentanoyl)
cRGD-PEG6-MMAE (Ligand=Cyclic(RGDfC); Substrate=PEG6-o; o=5-amino-3-oxopentanoyl)
cRAD-MMP-MMAE (Ligand=Cyclic(RADfC); Substrate=o-PLGC(Me)AG-o; o=5-amino-3-oxopentanoyl)
Suc-eeeeeeeee-XDPRSFL-rrrrrrrrr-c(Cy5)-CONH2
Suc-eeeeeeeee-ODPRSFL-rrrrrrrrr-c(Cy5)-CONH2
Suc-eeeeeeeee-Xdprsfl-rrrrrrrrr-c(Cy5)-CONH2
cRGD-ACPP-MMAE
(rrrrrrrrr)-Cy5 CPP-c(RGDfK)-(eeeeeeeee)
EDDDDKA-aca-R9-aca-C(Fl)-CONH2
Fl-aca-CRRRRRRRRR-aca-EEEEEEEEEC-CONH2
Fl-aca-CEEEE-aca-RRRRRRRRRC-CONH2
H2N-EEEEEDDDDKA-aca-RRRRRRRRR-aca-C(Fl)-CONH2
H2N-EDDDDKA-aca-RRRRRRRRR-aca-C(Fl)-CONH2
H2N-EEEEEDDDDK ARRRRRRRRR-aca-C(Fl)-CONH2
H2N-EEDDDDKA-aca-rrrrrrrrr-aca-C(Fl)-CONH2
H2N-DDDDDDKARRRRRRRRR-aca-C(Fl)-CONH2
H2N-EEDDDDKAR-aca-RR-aca-RR-aca-RR-aca-RR-aca-C(Fl)-CONH2
H2N-eeeeee-aca-PLGLAG-rrrrrrr-aca-c(Fl)-CONH2
EDA-aca-R,-aca-C(Fl)-CONH2
EDDDDKA-aca-R6-aca-C(DOX)-CONH2
EEEDDDEEEDA-aca-R9-aca-Y(12SI)-CONH2
ededdAAeeeDDDDKA-aca-R,-aca-C(Fl)-CONH2
eddedededDDDDKA-aca-Rs-AGA-R6-aca-C(DOX)-CONH2
Ggedgddeeeeeeddeed-aca-PLGLAG-aca-R8-AAA-Ri2-aca-C(Fl)-CONH2
eeddeeddKA-aca-R7-aca-C(Fl)-CONH2
eDDDDKA-aca-RGRGRRR-aca-C(Fl)-CONH2
eddddeeeeeee-aca-PLGLAGKA-aca-R10-aca-C(Fl)-CONH2
eeeeeeeeeeeeeeee-aca-DDDDKA-aca-R20-aca-C(Fl)-CONH2
eeeeeeeeeddddd-aca-DDDDK A-aca-R, 7-aca-Y(′ 2<iI)-CONH2
dddddddddddddddd-aca-PLGLAG-aca-R,4-aca-C(DOX)-CONH2
NH2-eeeeee-ahx-PLG
LAG-rrrrrrrrr-ahx-c(Fl)-CONH2, where “ahx” indicates ammohexanoic acid
EEEEEDDDDKAXRRRRRRRRRXC(FI)
EEEEEDDDDKARRRRRRRRRXC(Fl)
EDDDDKAXRRRRRRRRRXC(Fl)
EEDDDDKARXRRXRRXRRXRRXC(Fl)
DDDDDDKARRRRRRRRRXC(Fl)
EEDDDDKAXrrrrrrrrrXC(Fl)
eeeeeeXPLGLAGrrrrrrrrrXc(Fl)
UeeeeeeeeXPLGLAGrrrrrrrrrXk(Fl)
eeeeeeXPLGLAGrrrrrrrrrXc(Cy5)
UeeeeeeXPLGLAGrrrrrrrrrXc(Cy5)
UeeeeeeeeXPLGLAGπτrrrrrrXk(Cy5)
11-kDa PEG]XeeeeeeeeeXPLGLAGrrrrrrrrrXk(Cy5)
11-kDa PEG]XeeeeeeeeeXLALGPGrrrrrrrrrXk(Cy5)
Fl-XrrrrrrrrrXPLGLAGeeeeeeee-βAla
Fl-XrrrrrrrrrXSGRSAeeeeeeee-βAla
eeeeeeXSGRSAXrrrrrrrrrXc(Cy5)
Fl-rrrrrrrrrc-SS-ceeeeee
succinyl-e8-XPLGLAG-r9-Xk, where X denotes 6-aminohexanoyl
[11 kDa PEG]-X-e9-XPLGLAG-r9
[11-kDa PEG]-X-e9-XPLGLAG-r9-Xk(Cy5)
H2N-e6-XPLGLAG-r9-Xc(Cy5)-CONH2, where X=aminohexanoic acid
H2N-eeeeee-(ahx)-PLG LAG-rrrrrrrrr-(ahx)-c(Fluor)-CONH2
XeeeeeeeeeXPLGLAGrrrrrrrrXk
eeeeeeeeeXLALGPG-rrrrrrrrrXk(Cy5)
mPEG(1 lkd)-S-CH2-CONH-ahx-e9-ahx-PLGLAG-r9-ahx-k-CONH2 mPEG-S-CH2CONH-e9-ahx-PLGLAG-r9-K[DOTA(Gd)]-CONH2
(11 KDa-mPEG)-e9-XPLGLAG-r9-[DPK-99mTc(CO)3]
(70 KDa-dextran)-e9-XPLGLAG-r9-[DPK-99mTc(CO)3]
murine serum albumin)-e9-XPLGLAG-r9-[DPK-99mTc(CO)3],
(PAMAM generation 5 dendrimer)-e9-XPLGLAX-r9-[DPK-′mTc(CO)3]
(70 KDa dextran)-e9-XPLGLAX-r9-(DOTA-′ 11In)
(11-KDa-mPEG)-e9-XPLGLAG-r9-K(DOTA-Gd)
Suc9-(70 KDa dextran)-e9-XPLGLAG-r9-K(DOT A-Gd)
Suc9-(70 KDa dextran)-e9-XPLGLAX-r9-K(DOT A-Gd)
Suc9-(70 KDa dextran)-e9-XPLGLAG-r9-K(DOT A-Gd)
cyclic[succinoyl-PLGLAG-c(11 KDa-mPEG)-e9-XPLGLAG-r9-K]-k(Cy5)
Cy5-X-e6-XPLGLAG-r9-Xk(Cy5)
Cy7-X-e6-XPLGLAG-r9-Xk(Cy5)
11 KDa mPEG-e9-PLGLAG-r9
Ac-r9-k-NH2
mPEG(11kd)-e9-XPLGLAG-r9-Xk-NH2
e9-XPLGLAG-r9-Xk-NH2

TABLE 1

| | | | | | | | | | | | | C- |
|---|---|---|---|---|---|---|---|---|---|---|---|---|
| Cap | Macromolecule | Polyanion | P4 | P3 | P2 | P1 | P1′ | P2′ | P3′ . . . Pn′ | Polycation | Cargo | term |
| Suc9 | Dextran | e9 | X | P | L | G | L | A | G | r9 | K[DOTA(Gd)] | NH₂ |
| | (70 KDa) | | | | | | | | | | | |
| Suc | — | e9 | X | P | L | G | C(Me) | A | X | r9 | DPK | NH₂ |
| Suc | — | e9 | X | P | ThienylAla | G | C(Me) | A | X | r9 | DPK | NH₂ |
| Suc | — | e9 | X | P | F(4-Cl) | G | C(Me) | A | X | r9 | DPK | NH₂ |
| Suc | — | e8 | X | P | L | G | L | A | G | r9 | c[Cy5] | NH₂ |
| Suc | — | e8 | X | P | F(4-Cl) | G | C(Me) | M | X | r9 | c[Cy5] | NH₂ |
| Suc | — | e8 | X | P | F(4-Cl) | G | C(Me) | Y | X | r9 | c[Cy5] | NH₂ |
| Suc | — | e8 | X | P | F(4-Cl) | G | C(Me) | R | X | r9 | c[Cy5] | NH₂ |
| Suc | — | e8 | X | P | F(4-Cl) | G | C(Me) | PhGly | X | r9 | c[Cy5] | NH₂ |
| Suc | — | e8 | X | P | F(4-Cl) | G | C(Me) | C(Me) | X | r9 | c[Cy5] | NH₂ |
| — | Albumin | e9 | X | P | L | G | L | A | X | r9 | DPK | NH₂ |
| Suc | — | e8 | X | P | C(Me) | G | C(Me) | A | X | r9 | c[Cy5] | NH₂ |
| Suc | — | e8 | X | P | ThienylAla | G | C(Me) | A | X | r9 | c[Cy5] | NH₂ |
| Suc | — | e8 | X | P | F(4-Cl) | G | C(Me) | A | X | r9 | c[Cy5] | NH₂ |
| Suc | — | e8 | X | P | K(Dnp) | G | C(Me) | A | X | r9 | c[Cy5] | NH₂ |
| — | Albumin | e9 | X | P | L | G | L | A | X | r9 | DPK | NH₂ |
| Suc | — | e8 | X | P | L | G | C(Me) | M | X | r9 | c[Cy5] | NH₂ |
| Suc | — | e8 | X | P | L | G | C(Me) | Y | X | r9 | c[Cy5] | NH₂ |
| Suc127 | PAMAM-Gen5 | e9 | X | P | L | G | L | A | X | r9 | DPK | NH₂ |
| Suc | — | e8 | X | P | L | G | C(Me) | A | X | r9 | c[Cy5] | NH₂ |
| Suc9 | Dextran | e9 | X | P | L | G | L | A | G | r9 | K[DOTA(Gd)] | NH₂ |
| | (70 KDa) | | | | | | | | | | | |
| Suc127 | PAMAM-Gen5 | e9 | X | P | L | G | L | A | X | r9 | k[Cy5] | NH₂ |
| — | — | — | — | — | — | — | — | — | — | r9 | Xe[Cy5] | NH₂ |
| Ac127 | PAMAM-Gen5 | e9 | X | P | L | G | L | A | X | r9 | k[Cy5] | NH₂ |
| Suc | — | e8 | X | P | L | G | L | F(4- | A | Xr9 | k[Cy5] | NH₂ |
| | | | | | | | | NO2) | | | | |
| Suc127 | PAMAM-Gen5 | e9 | X | P | L | G | L | A | X | r9 | k[Cy5] | NH₂ |
| Suc63 | PAMAM-Gen4 | e9 | X | P | L | G | L | A | X | r9 | k[Cy5] | NH₂ |
| — | Albumin | e9 | X | P | L | G | L | A | G | r9 | DPK | NH₂ |
| Suc136 | Dextran | e9 | X | P | L | G | L | A | X | r9 | k[Cy5] | NH₂ |
| | (86 KDa) | | | | | | | | | | | |
| Suc | — | e8 | X | P | L | G | L | A | X | r9 | k[Cy5] | NH₂ |
| Suc9 | Dextran | e9 | X | P | L | G | L | A | X | r9 | K[DOTA(Gd)] | NH₂ |
| | (70 KDa) | | | | | | | | | | | |
| Suc9 | Dextran | e9 | X | P | L | G | L | A | X | r9 | K[DOTA(Gd)] | NH₂ |
| | (70 KDa) | | | | | | | | | | | |
| Suc9 | Dextran | e9 | X | P | L | G | L | A | G | r9 | k[Cy5] | NH₂ |
| | (70 KDa) | | | | | | | | | | | |
| Suc9 | Dextran | e9 | X | P | L | G | L | A | G | r9 | DPK | NH₂ |
| | (70 KDa) | | | | | | | | | | | |
| Suc | — | e8 | X | p | l | g | l | a | g | r9 | k[Cy5] | NH₂ |
| — | Albumin | e9 | X | p | l | g | l | a | g | r9 | k[Cy5] | NH₂ |
| Suc9 | Dextran | e9 | X | p | l | g | l | a | g | r9 | k[Cy5] | NH₂ |
| Suc97 | Dextran | e9 | X | P | L | G | L | A | G | r9 | k[Cy5] | NH₂ |
| | (500 KDa) | | | | | | | | | | | |
| — | Albumin | e9 | X | P | L | G | L | A | G | r9 | k[Cy5] | NH₂ |
| Suc9 | Dextran | e9 | X | P | L | G | L | A | G | r9 | k[Cy5] | NH₂ |
| | (70 KDa) | | | | | | | | | | | |
| — | Albumin | e9 | X | P | L | G | L | A | G | r9 | k[Cy5] | NH₂ |
| Suc | | e8 | X | P | L | G | L | A | X | r9 | k[Cy5] | NH₂ |
| Suc9 | Dextran | e9 | X | P | L | G | L | A | G | r9 | k[Cy5] | NH₂ |
| | (70 KDa) | | | | | | | | | | | |
| — | Albumin | e9 | X | P | L | G | L | A | G | r9 | k[Cy5] | NH₂ |
| — | Albumin | e9 | X | P | L | G | L | A | G | r9 | k[Cy5] | NH₂ |
| — | Albumin | e9 | X | P | L | G | L | A | G | r9 | k[Cy5] | NH₂ |
| Suc9 | Dextran | e9 | X | P | L | G | L | A | G | r9 | k[Cy5] | NH₂ |
| | (70 KDa) | | | | | | | | | | | |
| Suc | nonconj. | e8 | X | P | L | G | L | A | G | r9X | k[Cy5] | NH₂ |
| | Albumin | | | | | | | | | | | |
| Suc | — | e8 | X | P | L | G | L | A | G | r9X | k[Cy5] | NH₂ |
| — | Albumin | e9 | X | P | L | G | L | A | G | r9 | k[Cy5] | NH₂ |
| — | mPEG | e9 | x | p | l | g | l | a | g | r9X | k[Cy5] | NH₂ |
| | (5 KDa) | | | | | | | | | | | |
| — | mPEG | e9 | X | P | L | G | L | A | G | r9 | K[DOTA(Gd)] | NH₂ |
| | (11 KDa) | | | | | | | | | | | |
| — | mPEG | e10 | X | P | L | G | F(4- | A | Q | Xr9 | k[Cy5] | NH₂ |
| | (11 KDa) | | | | | | NO₂) | | | | | |
| — | mPEG | e10 | X | P | L | G | C(Me) | W | A | Qr9 | k[Cy5] | NH₂ |
| | (11 KDa) | | | | | | | | | | | |
| — | mPEG | e9 | X | P | L | G | C(Me) | W | A | Qr9 | k[Cy5] | NH₂ |
| | (11 KDa) | | | | | | | | | | | |

In some embodiments, cargo C and/or compound T (e.g., radiosensitizing agent T) may be or comprise a fluorescent molecule such as fluorescein. Fluorescent cargo moieties enable easy measurement by fluorescence microscopy or flow cytometry in unfixed cultured cells. However, oligoarginine sequences, such as make up portion B, have been demonstrated to import a very wide varieties of cargoes C and/or compounds T, ranging from small polar molecules to nanoparticles and vesicles (Tung & Weissleder, Advanced Drug Delivery Reviews 55: 281-294 (2003)). Thus, in embodiments of the invention, a cargo portion C and/or compound T (e.g., radiosensitizing agent T) is any suitable cargo moiety capable of being taken up by a cell while connected to a basic portion B.

For example, for in vivo imaging purposes, C and/or T may be labeled with a positron-emitting isotope (e.g. ¹⁸F) for positron emission tomography (PET), gamma-ray isotope (e.g. ^{99m}Tc) for single photon emission computed tomography (SPECT), a paramagnetic molecule or nanoparticle (e.g. Gd³⁺ chelate or coated magnetite nanoparticle) for magnetic resonance imaging (MM), a near-infrared fluorophore for near-infra red (near-IR) imaging, a luciferase (firefly, bacterial, or coelenterate) or other luminescent molecule for bioluminescence imaging, or a perfluorocarbon-filled vesicle for ultrasound. For therapeutic purposes, for example, suitable classes of cargo C and/or compound T (e.g., radiosensitizing agent T) include but are not limited to: a) chemotherapeutic agents such as doxorubicin, mitomycin, paclitaxel, nitrogen mustards, etoposide, camptothecin, 5-fluorouracil, etc.; b) radiation sensitizing agents such as porphyrins for photodynamic therapy, or ¹⁰B clusters or ¹⁵⁷Gd for neutron capture therapy; or c) peptides or proteins that modulate apoptosis, the cell cycle, or other crucial signaling cascades. Existing chemotherapeutic drugs may be used, although they may not be ideal, because they have already been selected for some ability to enter cells on their own. In some embodiments of the molecules of the invention, cargoes that are unable to enter or leave cells without the help of the polybasic portion B may be employed.

In some embodiments, cargo portion C and/or compound T (e.g., radiosensitizing agent T) include a fluorescent moiety, such as a fluorescent protein, peptide, or fluorescent dye molecule. Common classes of fluorescent dyes include, but are not limited to, xanthenes such as rhodamines, rhodols and fluoresceins, and their derivatives; bimanes; coumarins and their derivatives such as umbelliferone and aminomethyl coumarins; aromatic amines such as dansyl; squarate dyes; benzofurans; fluorescent cyanines; carbazoles; dicyanomethylene pyranes, polymethine, oxabenzanthrane, xanthene, pyrylium, carbostyl, perylene, acridone, quinacridone, rubrene, anthracene, coronene, phenanthrecene, pyrene, butadiene, stilbene, lanthanide metal chelate complexes, rare-earth metal chelate complexes, and derivatives of such dyes. Fluorescent dyes are discussed, for example, in U.S. Pat. No. 4,452,720; U.S. Pat. No. 5,227,487 and U.S. Pat. No. 5,543,295. In some embodiments, cargo C and/or compound T (e.g., radiosensitizing agent T) includes detection agents.

In some embodiments, a cargo portion C and/or a compound T (e.g., radiosensitizing agent T) may include a fluorescein dye. Typical fluorescein dyes include, but are not limited to, 5-carboxyfluorescein, fluorescein-5-isothiocyanate and 6-carboxyfluorescein; examples of other fluorescein dyes can be found, for example, in U.S. Pat. No. 6,008,379; U.S. Pat. No. 5,750,409; U.S. Pat. No. 5,066,580 and U.S. Pat. No. 4,439,356. In some embodiments, a cargo portion C and/or compound T (e.g., radiosensitizing agent T) includes a rhodamine dye, such as, for example, tetramethylrhodamine-6-isothiocyanate, 5-carboxytetramethyrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride (sold under the tradename of TEXAS RED®), and other rhodamine dyes. Other rhodamine dyes can be found, for example, in U.S. Pat. No. 6,080,852, U.S. Pat. No. 6,025,505, U.S. Pat. No. 5,936,087, U.S. Pat. No. 5,750,409. In some embodiments, a cargo portion C and/or compound T includes a cyanine dye, such as, for example, Cy3, Cy3B, Cy3.5, Cy5, Cy5.5, Cy 7, Alexa 647, IRDye-700DX and IRDYE 800CW.

Some of the above compounds or their derivatives will produce phosphorescence in addition to fluorescence, or will only phosphoresce. Some phosphorescent compounds include porphyrins, phthalocyanines, polyaromatic compounds such as pyrenes, anthracenes and acenaphthenes, and so forth, and may be, or may be included in, a cargo portion C. In some embodiments, a cargo portion C and/or compound includes a fluorescence quencher, such as, for example, a (4-dimethylamino-phenylazo)benzoic acid (DABCYL) group.

In some embodiments, a cargo moiety and/or compound T is all or part of a molecular beacon. In some embodiments a cargo moiety C and/or compound T is combined with a quencher moiety Q to form all or part of a molecular beacon. In some embodiments a cargo moiety C is combined with a quencher moiety Q (in some embodiments Q includes compound T) to form all or part of a molecular beacon. One or both of the complementary regions may be part of the cargo moiety. Where only one of the complementary regions (e.g., the fluorescent moiety) is part of the cargo moiety, and where the quencher moiety is part of the linker X and/or Y or the acidic portion A, then cleavage of the linker X and/or Y will allow fluorescence of the fluorescent portion and detection of the cleavage. Upon cellular uptake, the fluorescent portion of a molecular beacon will allow detection of the cell. For example, a quencher Q may be attached to an acidic portion A to form a MTS molecule having features of the MTS molecules of the present disclosure where cargo is fluorescent and is quenched by Q. The quenching of the cargo moiety by Q is relieved upon cleavage of X and/or Y, allowing fluorescent marking of a cell taking up portion B with cargo C and/or compound T. The combination of fluorescence quenching and selective uptake should increase contrast between tissues able to cleave X and/or Y compared to those that cannot cleave X and/or Y.

A pair of compounds may be connected to form a molecular beacon or FRET pair, having complementary regions with a fluorophore and a fluorescent quencher associated together so that the fluorescence of the fluorophore is quenched by the quencher. Such pairs can be useful as detection agents and any fluorescent pairs known or described herein can be employed with the present invention. One or both of the complementary regions may be part of the cargo portion C and/or compound T. Where only one of the complementary regions (e.g., the fluorescent moiety) is part of the cargo portion C and/or compound T, and where the quencher moiety is part of the linker X and/or Y or the acidic portion A, then cleavage of the linker X and/or Y will allow fluorescence of the fluorescent portion and detection of the cleavage. Upon cellular uptake, the fluorescent portion of a molecular beacon will allow detection of the cell. For example, a quencher Q may be attached to an acidic portion A to form a MTS molecule having features of the molecules as described in this disclosure where cargo C and/or compound T is fluorescent and is quenched by Q. The quenching of C and/or T by Q is relieved upon cleavage of X and/or Y, allowing fluorescent marking of a cell taking up portion B comprising C and/or T. The combination of fluorescence quenching and selective uptake should increase contrast between tissues able to cleave X and/or Y compared to those that cannot cleave X and/or Y.

In some embodiments, C, T and/or Q comprise all or part of a donor:acceptor FRET pair or a BRET (bioluminescence resonance energy transfer) pair. Donors can include any appropriate molecules listed herein or known in the art and as such include but are not limited to FITC; Cy3; EGFP; cyan fluorescent protein (CFP); EGFP; 6-FAM; fluorescein, IAEDANS, EDANS and BODIPY FL. Acceptors can include any appropriate molecules listed herein or known in the art and as such include but are not limited to TRITC; Cy5; Cy3; YFP; 6-FAM; LC Red 640; Alexa Fluor 546; fluorescein; tetramethylrhodamine; Dabcyl (acceptor); BODIPY FL; QSY 7, QSY 9, QSY 21 and BBQ-650 dyes. Exemplary FRET pairs can include but are not limited to CFP:YFP; 6-FAM:Cy5; Cy5:Cy7; Cy5:IRdye800CW; FITC:TRITC; Cy3:Cy5; EGFP:Cy3; EGFP:YFP; 6-FAM:LC Red 640 or Alexa Fluor 546; fluorescein:tetramethylrhodamine; IAEDANS:fluorescein; EDANS:Dabcyl; fluorescein:fluorescein; BODIPY FL:BODIPY FL; and fluorescein:QSY 7 and QSY 9 dyes.

In some embodiments, the cargo moiety C, compound T and/or quencher moiety Q are or comprise a fluorescent moiety including but not limited to a fluorescent protein, peptide, or fluorescent dye molecule. Common classes of fluorescent dyes include, but are not limited to, xanthenes such as rhodamines, rhodols and fluoresceins, and their derivatives; bimanes; coumarins and their derivatives such as umbelliferone and aminomethyl coumarins; aromatic amines such as dansyl; squarate dyes; benzofurans; fluorescent cyanines; carbazoles; dicyanomethylene pyranes, polymethine, oxabenzanthrane, xanthene, pyrylium, carbostyl, perylene, acridone, quinacridone, rubrene, anthracene, coronene, phenanthrecene, pyrene, butadiene, stilbene, lanthanide metal chelate complexes, rare-earth metal chelate complexes, and derivatives of such dyes. Fluorescent dyes are discussed, for example, in U.S. Pat. No. 4,452,720; U.S. Pat. No. 5,227,487; and U.S. Pat. No. 5,543,295.

In some embodiments, a cargo moiety C and/or quencher moiety Q are or comprise fluorescein dyes. Typical fluorescein dyes include, but are not limited to, 5-carboxyfluorescein, fluorescein-5-isothiocyanate and 6-carboxyfluorescein; examples of other fluorescein dyes can be found, for example, in U.S. Pat. No. 6,008,379, U.S. Pat. No. 5,750,409, U.S. Pat. No. 5,066,580, and U.S. Pat. No. 4,439,356. A cargo moiety C may include a rhodamine dye, such as, for example, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride (sold under the tradename of TEXAS RED®), and other rhodamine dyes. Other rhodamine dyes can be found, for example, in U.S. Pat. No. 6,080,852; U.S. Pat. No. 6,025,505; U.S. Pat. No. 5,936,087; U.S. Pat. No. 5,750,409. In some embodiments, a cargo moiety C and/or a compound T includes a cyanine dye, such as, for example, Cy3, Cy3B, Cy3.5, Cy5, Cy5.5, Cy 7.

In some embodiments, cargo moiety C, compound T and/or quencher moiety Q are or comprise fluorophores. Fluorophores are commercially available and any known and/or commercially available fluorophore can be employed as the cargo moiety C and/or compound T detectable entity for the present invention. In some embodiments, the fluorophore exhibits green fluorescence (such as for example 494 nm/519 nm), orange fluorescence (such as for example 554 nm/570 nm), red fluorescence (such as for example 590 nm/617 nm), or far red fluorescence (such as for example 651 nm/672 nm) excitation/emission spectra. In some embodiments, the fluorophore is a fluorophore with excitation and emission spectra in the range of about 350 nm to about 775 nm. In some embodiments the excitation and emission spectra are about 346 nm/446 nm, about 494 nm/519 nm, about 554 nm/570 nm, about 555 nm/572 nm, about 590 nm/617 nm, about 651 nm/672 nm, about 679 nm/702 nm or about 749 nm/775 nm. In some embodiments, the fluorophore can include but is not limited to AlexaFluor 3, AlexaFluor 5, AlexaFluor 350, AlexaFluor 405, AlexaFluor 430, AlexaFluor 488, AlexaFluor 500, AlexaFluor 514, AlexaFluor 532, AlexaFluor 546, AlexaFluor 555, AlexaFluor 568, AlexaFluor 594, AlexaFluor 610, AlexaFluor 633, AlexaFluor 647, AlexaFluor 660, AlexaFluor 680, AlexaFluor 700, and AlexaFluor 750 (Molecular Probes AlexaFluor dyes, available from Life Technologies, Inc. (USA)). In some embodiments, the fluorophore can include but is not limited to Cy dyes, including Cy2, Cy3, Cy3B, Cy3.5, Cy5, Cy5.5 and Cy7 (available from GE Life Sciences or Lumiprobes). In some embodiments the fluorophore can include but is not limited to DyLight 350, DyLight 405, DyLight 488, DyLight 550, DyLight 594, DyLight 633, DyLight 650, DyLight 680, DyLight 750 and DyLight 800 (available from Thermo Scientific (USA)). In some embodiments, the fluorophore can include but is not limited to a FluoProbes 390, FluoProbes 488, FluoProbes 532, FluoProbes 547H, FluoProbes 594, FluoProbes 647H, FluoProbes 682, FluoProbes 752 and FluoProbes 782, AMCA, DEAC (7-Diethylaminocoumarin-3-carboxylic acid); 7-Hydroxy-4-methylcoumarin-3; 7-Hydroxycoumarin-3; MCA (7-Methoxycoumarin-4-acetic acid); 7-Methoxycoumarin-3; AMF (4′-(Aminomethyl)fluorescein); 5-DTAF (5-(4,6-Dichlorotriazinyl)aminofluorescein); 6-DTAF (6-(4,6-Dichlorotriazinyl)aminofluorescein); 6-FAM (6-Carboxyfluorescein), 5(6)-FAM cadaverine; 5-FAM cadaverine; 5(6)-FAM ethylenediamme; 5-FAM ethylenediamme; 5-FITC (FITC Isomer I; fluorescein-5-isothiocyanate); 5-FITC cadaverin; Fluorescein-5-maleimide; 5-IAF (5-Iodoacetamidofluorescein); 6-JOE (6-Carboxy-4′,5′-dichloro-2′,7′-dimethoxyfluorescein); 5-CR1 10 (5-Carboxyrhodamine 110); 6-CR1 10 (6-Carboxyrhodamine 110); 5-CR6G (5-Carboxyrhodamine 6G); 6-CR6G (6-Carboxyrhodamine 6G); 5(6)-Carboxyrhodamine 6G cadaverine; 5(6)-Caroxyrhodamine 6G ethylenediamme; 5-ROX (5-Carboxy-X-rhodamine); 6-ROX (6-Carboxy-X-rhodamine); 5-TAMRA (5-Carboxytetramethylrhodamine); 6-TAMRA (6-Carboxytetramethylrhodamine); 5-TAMRA cadaverine; 6-TAMRA cadaverine; 5-TAMRA ethylenediamme; 6-TAMRA ethylenediamme; 5-TMR C6 maleimide; 6-TMR C6 maleimide; TR C2 maleimide; TR cadaverine; 5-TRITC; G isomer (Tetramethylrhodamine-5-isothiocyanate); 6-TRITC; R isomer (Tetramethylrhodamine-6-isothiocyanate); Dansyl cadaverine (5-Dimethylaminonaphthalene-1-(N-(5-aminopentyl))sulfonamide); EDANS C2 maleimide; fluorescamine; NBD; and pyrromethene and derivatives thereof.

Some of the above compounds or their derivatives will produce phosphorescence in addition to fluorescence, or will only phosphoresce. Some phosphorescent compounds include porphyrins, phthalocyanines, polyaromatic compounds such as pyrenes, anthracenes and acenaphthenes, and so forth, and in some embodiments are included in a compound T and/or a cargo moiety. In some embodiments, a cargo moiety and/or compound T include a fluorescence quencher, such as, for example, a (4-dimethylamino-phenylazo)benzoic acid (DABCYL) group.

In some embodiments, a cargo moiety and/or compound T is or comprises a fluorescent label. In some embodiments, a cargo moiety C, compound T and/or quencher moiety Q is indocarbocyanine dye, Cy5, Cy5.5, Cy7, IR800CW, or a combination thereof. In some embodiments, a cargo moiety is a MRI contrast agent. In some embodiments, a cargo moiety is Gd complex of [4,7, 10-tris(carboxymethyl)-1,4,7, 10-tetraazacyclododec-1-yl] acetyl.

In some embodiments, compound T (e.g., radiosensitizing agent T) and/or cargo C may include a chemotherapeutic moiety, such as a chemical compound useful in the treatment of cancer, or other therapeutic moiety, such as an agent useful in the treatment of ischemic tissue, or of necrotic tissue, or other therapeutic agent.

In some embodiments, compound T and/or cargo C may include a radiosensitization agent or moiety, such as a chemical compound useful in the radiosensitization of cancer.

Multiple membrane translocation signals (MTS) have been identified. For example, the Tat protein of the human immunodeficiency virus 1 (HIV-1) is able to enter cells from the extracellular environment. A domain from Antennapedia homeobox protein is also able to enter cells.

Molecules comprising a MTS may also be used to carry other molecules into cells along with them. The most important MTS are rich in amino acids such as arginine with positively charged side chains. Molecules transported into cell by such cationic peptides may be termed “cargo” or compound T (e.g., radiosensitizing agent T) and may be reversibly or irreversibly linked to the cationic peptides.

The uptake facilitated by molecules comprising a MTS can occur with specificity by including appropriate X and/or Y linkers as well as pre-targeting P moieties, enhancing uptake into most or all cells. It is desirable to have the ability to target the delivery of cargo to a type of cell, or to a tissue, or to a location or region within the body of an animal. Accordingly, we have identified a need for a MTS molecule with increased in vivo circulation.

In some embodiments, a MTS molecule disclosed herein has the formula according to one of the following:

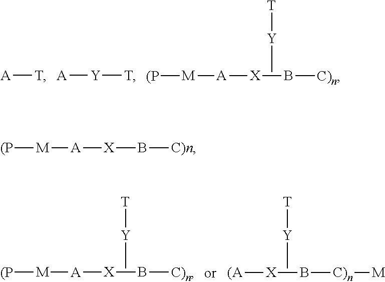

wherein C is a cargo moiety; T is a therapeutic compound; A is a peptide with a sequence comprising 5 to 9 consecutive acidic amino acids, wherein the amino acids are selected from: aspartates and glutamates; B is a peptide with a sequence comprising 5 to 20 consecutive basic amino acids; and X and/or Y is a linker that is cleavable by thrombin. In some embodiments of molecules having features of the invention, n is an integer between 1 and 15. In some embodiments of molecules having features of the invention, n is an integer between 1 and 10. In some embodiments of molecules having features of the invention, n is an integer between 1 and 5. In some embodiments of molecules having features of the invention, n is an integer between 1 and 4. In some embodiments of molecules having features of the invention, n is an integer between 1 and 3. In some embodiments of molecules having features of the invention, n is an integer between 1 and 2. In some embodiments of molecules having features of the invention, n is 1. In some embodiments of molecules having features of the invention, n is an integer of 1, 2, 3, 4, 5, 6, 7, 8, 9, 10, 11, 12, 13, 14, 15, 16, 17, 18, 19, or 20. In some embodiments of molecules having features of the invention, i is an integer between 1 and 20. In some embodiments of molecules having features of the invention, i is an integer between 1 and 10. In some embodiments of molecules having features of the invention, i is an integer between 1 and 5. In some embodiments of molecules having features of the invention, i is an integer between 1 and 4. In some embodiments of molecules having features of the invention, i is an integer between 1 and 3. In some embodiments of molecules having features of the invention, i is an integer between 1 and 2. In some embodiments of molecules having features of the invention, i is 1. In some embodiments of molecules having features of the invention, i is an integer of 1, 2, 3, 4, 5, 6, 7, 8, 9, 10, 11, 12, 13, 14, 15, 16, 17, 18, 19, or 20. In some embodiments, the T-Y pair is attached to either end of B.

In some embodiments, A is an antibody. In some embodiments, A is an anti-ErbB antibody. In some embodiments, A is an anti-HER2 antibody. In some embodiments, the antibody is an anti-EGFR antibody. In some embodiments, A is an antibody selected from the group consisting of cetuximab, trastuzumab, and pertruzumab. In some embodiments, A is cetuximab. In some embodiments, A is trastuzumab. In some embodiments, A is pertruzumab.

In some embodiments, a MTS molecule disclosed herein has the formula according to one of the following:

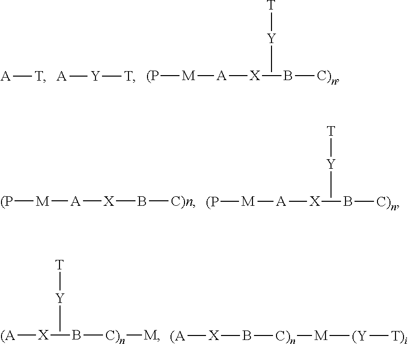

wherein C is a cargo moiety; T is a therapeutic moiety; A is a peptide with a sequence comprising 5 to 9 consecutive acidic amino acids, wherein the amino acids are selected from: aspartates and glutamates; B is a peptide with a sequence comprising 5 to 20 consecutive basic amino acids; and X and/or Y is a linker that is cleavable by thrombin. In some embodiments of molecules having features of the invention, n is an integer between 1 and 15. In some embodiments of molecules having features of the invention, n is an integer between 1 and 10. In some embodiments of molecules having features of the invention, n is an integer between 1 and 5. In some embodiments of molecules having features of the invention, n is an integer between 1 and 4. In some embodiments of molecules having features of the invention, n is an integer between 1 and 3. In some embodiments of molecules having features of the invention, n is an integer between 1 and 2. In some embodiments of molecules having features of the invention, n is 1. In some embodiments of molecules having features of the invention, n is an integer of 1, 2, 3, 4, 5, 6, 7, 8, 9, 10, 11, 12, 13, 14, 15, 16, 17, 18, 19, or 20. In some embodiments of molecules having features of the invention, i is an integer between 1 and 20. In some embodiments of molecules having features of the invention, i is an integer between 1 and 10. In some embodiments of molecules having features of the invention, i is an integer between 1 and 5. In some embodiments of molecules having features of the invention, i is an integer between 1 and 4. In some embodiments of molecules having features of the invention, i is an integer between 1 and 3. In some embodiments of molecules having features of the invention, i is an integer between 1 and 2. In some embodiments of molecules having features of the invention, i is 1. In some embodiments of molecules having features of the invention, i is an integer of 1, 2, 3, 4, 5, 6, 7, 8, 9, 10, 11, 12, 13, 14, 15, 16, 17, 18, 19, or 20. In some embodiments, the T-Y pair is attached to either end of B.

In some embodiments, A is an antibody. In some embodiments, A is an anti-ErbB antibody. In some embodiments, A is an anti-HER2 antibody. In some embodiments, the antibody is an anti-EGFR antibody. In some embodiments, A is an antibody selected from the group consisting of cetuximab, trastuzumab, and pertruzumab. In some embodiments, A is cetuximab. In some embodiments, A is trastuzumab. In some embodiments, A is pertruzumab.

In some embodiments, a MTS molecule disclosed herein has a formula according to one of the following:

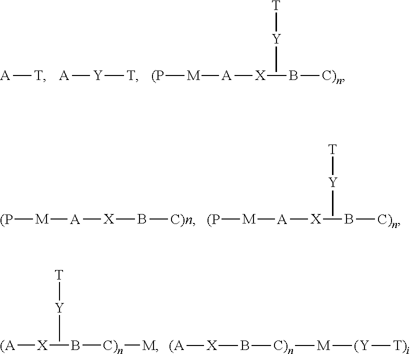

wherein C is a cargo moiety; T is a therapeutic compound; A is a peptide with a sequence comprising 5 to 9 consecutive acidic amino acids, wherein the amino acids are selected from: aspartates and glutamates; B is a peptide with a sequence comprising 5 to 20 consecutive basic amino acids; X and/or Y is a linker that is cleavable by thrombin; M is a macromolecular carrier; and n is an integer between 1 and 20. In some embodiments of molecules having features of the invention, n is an integer between 1 and 15. In some embodiments of molecules having features of the invention, n is an integer between 1 and 10. In some embodiments of molecules having features of the invention, n is an integer between 1 and 5. In some embodiments of molecules having features of the invention, n is an integer between 1 and 4. In some embodiments of molecules having features of the invention, n is an integer between 1 and 3. In some embodiments of molecules having features of the invention, n is an integer between 1 and 2. In some embodiments of molecules having features of the invention, n is 1. In some embodiments of molecules having features of the invention, n is an integer of 1, 2, 3, 4, 5, 6, 7, 8, 9, 10, 11, 12, 13, 14, 15, 16, 17, 18, 19, or 20. In some embodiments of molecules having features of the invention, i is an integer between 1 and 20. In some embodiments of molecules having features of the invention, i is an integer between 1 and 10. In some embodiments of molecules having features of the invention, i is an integer between 1 and 5. In some embodiments of molecules having features of the invention, i is an integer between 1 and 4. In some embodiments of molecules having features of the invention, i is an integer between 1 and 3. In some embodiments of molecules having features of the invention, i is an integer between 1 and 2. In some embodiments of molecules having features of the invention, i is 1. In some embodiments of molecules having features of the invention, i is an integer of 1, 2, 3, 4, 5, 6, 7, 8, 9, 10, 11, 12, 13, 14, 15, 16, 17, 18, 19, or 20. In some embodiments, the T-Y pair is attached to either end of B.

In some embodiments, A is an antibody. In some embodiments, A is an anti-ErbB antibody. In some embodiments, A is an anti-HER2 antibody. In some embodiments, the antibody is an anti-EGFR antibody. In some embodiments, A is an antibody selected from the group consisting of cetuximab, trastuzumab, and pertruzumab. In some embodiments, A is cetuximab. In some embodiments, A is trastuzumab. In some embodiments, A is pertruzumab.

Delivery of cargo C and/or compound T such as a fluorescent molecule may be used to visualize cells having a certain condition or cells in a region exhibiting a particular condition For example, thrombosis (clot formation) may be visualized by designing a linker X and/or Y to be cleaved by thrombin. Thus, fluorescent molecules are one example of a marker that may be delivered to target cells and regions upon release of a portion A upon cleavage of a linker X and/or Y.

In some embodiments, the cargo moiety and or compound T is selected from an imaging agent, a therapeutic agent, a lipid, a detection agent or a combination thereof.

In some embodiments, the cargo portion comprises at least two cargo moieties. In some embodiments, C comprises a marker cargo and a therapeutic cargo. Multiple cargo moieties can allow, for example, delivery of both a radioactive marker and an ultrasound or contrast agent, allowing imaging by different modalities. Alternatively, for example, delivery of radioactive cargo along with an anti-cancer agent, providing enhanced anticancer activity, or delivery of a radioactive cargo with a fluorescent cargo, allowing multiple means of localizing and identifying cells which have taken up cargo. Alternatively, delivery of a fluorescent or radioactive compound with a therapeutic compound can allow, for example, for identification of cells to which a therapeutic compound has been delivered.

In some embodiments, the compound T (e.g., radiosensitizing agent T) comprises at least two T moieties. In some embodiments, T comprises a marker cargo and a therapeutic compound. Multiple therapeutic moieties can allow, for example, delivery of both a radioactive marker and an ultrasound or contrast agent, allowing imaging by different modalities. Alternatively, for example, delivery of radioactive compound along with an anti-cancer agent, providing enhanced anticancer activity, or delivery of a radioactive cargo with a fluorescent cargo, allowing multiple means of localizing and identifying cells which have taken up compound. Alternatively, delivery of a fluorescent or radioactive compound with a therapeutic compound can allow, for example, for identification of cells to which a therapeutic compound has been delivered. In some embodiments, a portion A (such as an antibody) delivers the compound T (e.g., radiosensitizing agent T) to tumor. In some embodiments, a portion A (such as an antibody) delivers the compound T (e.g., radiosensitizing agent T) to tumor and the tumor is sensitized to the radiosensitizing agent T.

The cargo moiety is attached to B in any location or orientation. The cargo moiety need not be located at an opposite end of portion B than a linker X and/or Y. Any location of attachment of the cargo moiety to B is acceptable as long as that attachment remains after X is cleaved. For example, the cargo moiety may be attached to or near to an end of portion B with linker X and/or Y is attached to an opposite end of portion B. The cargo moiety may also be attached to or near to an end of portion B with linker X attached to or near to the same end of portion B.

In some embodiments, a cargo moiety C and/or compound T is a fluorescent molecule such as fluorescein. Fluorescent cargo moieties enable easy measurement by fluorescence microscopy or flow cytometry in unfixed cultured cells. In some embodiments, T is a therapeutic moiety capable of use in treating a variety of diseases, including inflammation, diabetes, cardiovascular diseases, neurodegenerative disorders and cancer. In some embodiments, T is a radiosensitizing agent. In some embodiments, the radiosensitizing agent is not an auristatin, including MMAE. In some embodiments, T is a maytansinoid. In some embodiments, T is mertansine.

In some embodiments, compound T and/or cargo C may include a radiosensitization agent or moiety, such as a chemical compound useful in the radiosensitization of cancer.

In some embodiments, a cargo moiety and/or compound T is labeled with a positron-emitting isotope (e.g., 18F) for positron emission tomography (PET), gamma-ray isotope (e.g., 99mTc) for single photon emission computed tomography (SPECT), a paramagnetic molecule or nanoparticle (e.g., Gd3+ chelate or coated magnetite nanoparticle) for magnetic resonance imaging (MRI), a near-infrared fluorophore for near-infra red (near-IR) imaging, a luciferase (firefly, bacterial, or coelenterate) or other luminescent molecule for bioluminescence imaging, or a perfluorocarbon-filled vesicle for ultrasound.

In some embodiments, cargo C and/or compound T includes a radioactive moiety, for example a radioactive isotope such as ²¹¹At, ¹³¹I, ¹²⁵I, ⁹⁰Y, ¹⁸⁶Re, ¹⁸⁸Re, ¹⁵³Sm, ²¹²Bi, ³²P, ⁶⁴Cu and ⁸⁹Zr. In some embodiments, a cargo moiety is a radioactive moiety, for example a radioactive isotope such as ²¹¹At, ¹³¹I, ¹²SI, ⁹⁰Y, ¹⁸⁶Re, ¹⁸⁸Re, ¹⁴⁸Sm, ¹³³Ba, ²¹²Bi, ¹²P, ³H, ¹⁴C, ³²P, ³⁵S, ⁸⁷Rb, ⁹⁰Sr, ¹¹⁵In, ¹²³Te, ¹³⁰Te, ¹³¹I, ¹³⁷Cs, ¹³⁸La, ¹⁴⁴Nd, ¹⁴⁷Sm, ¹⁴⁸Sm, ¹⁷⁶Lu, ¹⁸⁷Re, ¹⁸⁶Os, ²²²Rn, ²²⁶Ra, Barium-133, Cadmium-109, Cobalt-57, Cobalt-60, Europium-152, Manganese-54, Sodium-22, Zinc-65, Technetium-99m, Strontium-90, Thallium-204, Carbon-14, Tritium (Hydrogen-3), radioactive isotopes of Lu, Cu and Zr as well as others known to those of skill in the art.

In some embodiments, a cargo moiety and/or a therapeutic moiety are a therapeutic agent, such as a chemical compound useful in the treatment of cancer, ischemic tissue, or necrotic tissue.

For therapeutic purposes, for example, suitable classes of cargo moiety (C) and/or therapeutic moiety (T) include but are not limited to a) chemotherapeutic agents, b) radiation sensitizing agents, or c) peptides or proteins that modulate apoptosis, the cell cycle, or other crucial signaling cascades.

In some embodiments, a cargo moiety (C) or a therapeutic moiety (T) is or comprises an agent that treats a cardiovascular disorder. In some embodiments, the cargo moiety is a niacin, a fibrate, a statin, an Apo-Al mimetic peptide, an apoA-I transciptional up-regulator, an ACAT inhibitor, a CETP modulator, or a combination thereof, a Glycoprotein (GP) Ilb/IIIa receptor antagonist, a P2Y12 receptor antagonist, a Lp-PLA2-inhibitor, a leukotriene inhibitor, a MIF antagonist, or a combination thereof In some embodiments the cargo moiety is atorvastatin, cerivastatin, fluvastatin, lovastatin, mevastatin, pitavastatin, pravastatin, rosuvastatin, simvastatin, simvastatin and ezetimibe, lovastatin and niacin, extended-release, atorvastatin and amlodipine besylate, simvastatin and niacin, extended-release, bezafibrate, ciprofibrate, clofibrate, gemfibrozil, fenofibrate, DF4 (Ac-D-W-F-K-A-F-Y-D-K-V-A-E-K-F-K-E-A-F-NH2), DF5, RVX-208 (Resverlogix), avasimibe, pactimibe sulfate (CS-505), CI-1011 (2,6-diisopropylphenyl [(2, 4,6-triisopropylphenyl)acetyl]sulfamate), CI-976 (2,2-dimethyl-N-(2,4,6-trimethoxyphenyl)dodecanamide), VULM1457 (1-(2,6-diisopropyl-phenyl)-3-[4-(4′-mtrophenylthio)phenyl] urea), CI-976 (2,2-dimethyl-N-(2,4,6-trimethoxyphenyl)dodecanamide), E-5324 (n-butyl-N′-(2-(3-(5-ethyl-4-phenyl-1H-imidazol-1-yl)propoxy)-6-methylphenyl)urea), HL-004 (N-(2,6-diisopropylphenyl) tetradecylthioacetamide), KY-455 (N-(4,6-dimethyl-1-pentylindolin-7-yl)-2,2-dimethylpropanamide), FY-087 (N-[2-[N′-pentyl-(6,6-dimethyl-2,4-heptadiynyl)amino]ethyl]-(2-methyl-1-naphthyl-thio)acetamide), MCC-147 (Mitsubishi Pharma), F 1251 1 ((S)-2′,3′,5′-trimethyl-4′-hydroxy-alpha-dodecylthioacetanihde), SMP-500 (Sumitomo Pharmaceuticals), CL 277082 (2,4-difluoro-phenyl-N[[4-(2,2-dimethylpropyl)phenyl]methyl]-N-(hepthyl)urea), F-1394 ((1s,2s)-2 [3-(2,2-dimethylpropyl)-3-nonylureido]aminocyclohexane-1-yl 3-[N-(2,2,5,5-tetramethyl-1,3-dioxane-4-carbonyl)amino]propionate), CP-113818 (N-(2,4-bis(methylthio)-6-methylpyridm-3-yl)-2-(hexylthio)decanoic acid amide), YM-750, torcetrapib, anacetrapid, JTT-705 (Japan Tobacco/Roche), abciximab, eptifibatide, tirofiban, roxifiban, variabihn, XV 459 (N(3)-(2-(3-(4-formamidinophenyl)isoxazolm-5-yl)acetyl)-N(2)-(1-butyloxycarbonyl)-2,3-diaminopropionate), SR 121566A (3-[N-{4-[4-(aminoiminomethyl)phenyl]-1,3-thiazol-2-yl J-N-(I-carboxymethylpiperid-4-yl) aminol propionic acid, trihydrochloride), FK419 ((S)-2-acetylamino-3-[(R)-[1-[3-(piperidin-4-yl) propionyl] piperidin-3-ylcarbonyl] ammo] propionic acid trihydrate), clopidogrel, prasugrel, cangrelor, AZD6140 (AstraZeneca); MRS 2395 (2,2-Dimethyl-propionic acid 3-(2-chloro-6-methylaminopurin-9-y])-2-(2,2-dimethyl-propionyloxymethyl)-propyl ester); BX 667 (Berlex Biosciences); BX 048 (Berlex Biosciences); darapladib (SB 480848); SB-435495 (GlaxoSmithKline); SB-222657 (GlaxoSmithKline); SB-253514 (GlaxoSmithKline); A-81834 (3-(3-(1,1-dimethylethylthio-5-(quinoline-2-ylmethoxy)-1-(4-chloromethylphenyl)indole-2-yl)-2,2-dimethylpropionaldehyde oxime-O-2-acetic acid; AME103 (Amira); AME803 (Amira); atreleuton; BAY-x-1005 ((R)-(+)-alpha-cyclopentyl-4-(2-quinolinylmethoxy)-Benzeneacetic acid); CJ-13610 (4-(3-(4-(2-Methyl-imidazol-1-yl)-phenylsulfanyl)-phenyl)-tetrahydro-pyran-4-carboxylic acid amide); DG-031 (DeCode); DG-051 (DeCode); MK886 (1-[(4-chlorophenyl)methyl]3-[(1,1-dimethylethyl)thio]-α,α-dimethyl-5-(1-methylethyl)-1H-indole-2-propanoic acid, sodium salt); MK591 (3-(1-4[(4-chlorophenyl)methyl]-3-[(t-butylthio)-5-((2-quinoly)methoxy)-1H-indole-2]-, dimethylpropanoic acid); RP64966 ([4-[5-(3-Phenyl-propyl)thiophen-2-yl]butoxy] acetic acid); SA6541 ((R)-S-[[4-(dimethylamino)phenyl]methyl]-N-(3-mercapto-2methyl-1-oxopropyl-L-cysteine); SC-56938 (ethyl-1-[2-[4-(phenylmethyl)phenoxy] ethyl]-4-piperidine-carboxylate); VIA-2291 (Via Pharmaceuticals); WY-47,288 (2-[(1-naphthalenyloxy)methyl]quinoline); zileuton; ZD-2138 (6-((3-fluoro-5-(tetrahydro-4-methoxy-2H-pyran-4yl)phenoxy)methyl)-]-methyl-2(1H)-quinlolinone); or combinations thereof.

In some embodiments, a cargo moiety (C) or a therapeutic moiety (T) is or comprises a drug. In some embodiments, the drug is an agent that modulates death (e.g., via apoptosis or necrosis or modulates radiosensitization) of a cell. In some embodiments, the drug is a cytotoxic agent. In some embodiments, the drug is maytansine, or a maytansinoid (including but not limited to mertansine), methotrexate (RHEUMATREX®, Amethopterin); cyclophosphamide (CYTOXAN®); thalidomide (THALIDOMID®); paclitaxel; pemetrexed; pentostatin; pipobroman; pixantrone; plicamycin; procarbazine; proteasome inhibitors (e.g., bortezomib); raltitrexed; rebeccamycin; rubitecan; SN-38; salinosporamide A; satraplatin; streptozotocin; swainsonine; tariquidar; taxane; tegafur-uracil; temozolomide; testolactone; thioTEPA; tioguanine; topotecan; trabectedin; tretinoin; triplatin tetranitrate; tris(2-chloroethyl)amine; troxacitabine; uracil mustard; valrubicin; vinblastine; vincristine; vinorelbine; vorinostat; zosuquidar; or a combination thereof. In some embodiments, the drug is a pro-apoptotic agent. In some embodiments, the drug is an anti-apoptotic agent. In some embodiments, the drug is selected from: minocycline; SB-203580 (4-(4-Fluorophenyl)-2-(4-methylsulfinyl phenyl)-5-(4-pyridyl) 1H-imidazole); PD 169316 (4-(4-Fluorophenyl)-2-(4-nitrophenyl)-5-(4-pyridyl)-1H-imidazole); SB 202190 (4-(4-Fluorophenyl)-2-(4-hydroxyphenyl)-5-(4-pyridyl)1H-imidazole); RWJ 67657 (4-[4-(4-fluorophenyl)-1-(3-phenylpropyl)-5-(4-pyridinyl)-1H-imidazol-2-yl]-3-butyn-1-ol); SB 220025 (5-(2-Amino-4-pyrimidinyl)-4-(4-fluorophenyl)-1-(4-piperidinlyl)imidazole); D-JNKI-I ((D)-hJIP175-157-DPro-DPro-(D)-HIV-TAT57-48); AM-111 (Auris); SP600125 (anthra[1,9-cd]pyrazol-6(2H)-one); JNK Inhibitor I ((L)-HIV-T AT48-57-PP-JBD20); JNK Inhibitor III ((L)-HIV-T AT47-57-gaba-c-Junδ33-57); AS601245 (1,3-benzothiazol-2-yl (2-[[2-(3-pyridinyl) ethyl] amino]-4 pyrimidinyl) acetonitrile); JNK Inhibitor VI (H2N-RPKRPTTLNLF-NH2); JNK Inhibitor VIII (N-(4-Amino-5-cyano-6-ethoxypyridin-2-yl)-2-(2,5-dimethoxyphenyl)acetamide); JNK Inhibitor IX (N-(3-Cyano-4,5,6,7-tetrahydro-1-benzothien-2-yl)-1-naphthamide); dicumarol (3,3′-Methylenebis(4-hydroxycoumarin)); SC-236 (4-[5-(4-chlorophenyl)-3-(trifluoromethyl)-1H-pyrazol-1-yl]benzene-sulfonamide); CEP-1347 (Cephalon); CEP-11004 (Cephalon); an artificial protein comprising at least a portion of a Bcl-2 polypeptide; a recombinant FNK; V5 (also known as Bax inhibitor peptide V5); Bax channel blocker ((±)-1-(3,6-Dibromocarbazol-9-yl)-3-piperazin-1-yl-propan-2-ol); Bax inhibiting peptide P5 (also known as Bax inhibitor peptide P5); Kp7-6; FAIM(S) (Fas apoptosis inhibitory molecule-short); FAIM(L) (Fas apoptosis inhibitory molecule-long); Fas:Fc; FAP-I; NOK2; F2051; Fl 926; F2928; ZB4; Fas M3 mAb; EGF; 740 Y-P; SC 3036 (KKHTDDGYMPMSPGVA); PI 3-kinase Activator (Santa Cruz Biotechnology, Inc.); Pam3Cys ((S)-(2,3-bis(palmitoyloxy)-(2RS)-propyl)-N-palmitoyl-(R)-Cys-(S)-Ser(S)-Lys4-OH, trihydrochloride); Actl (NF-kB activator 1); an anti-IkB antibody; Acetyl-11-keto-b-Boswellic Acid; Andrographolide; Caffeic Acid Phenethyl Ester (CAPE); Gliotoxin; Isohelenin; NEMO-Binding Domain Binding Peptide (DRQIKIWFQNRRMKWKKTALDWSWLQTE); NF-kB Activation Inhibitor (6-Amino-4-(4-phenoxyphenylethylamino)quinazoline); NF-kB Activation Inhibitor II (4-Methyl-Nl-(3-phenylpropyl)benzene-1,2-diamine); NF-kB Activation Inhibitor III (3-Chloro-4-nitro-N-(5-nitro-2-thiazolyl)-benzamide); NF-kB Activation Inhibitor IV ((E)-2-Fluoro-4′-methoxystilbene); NF-kB Activation Inhibitor V (5-Hydroxy-(2,6-diisopropylphenyl)-1H-isoindole-1,3-dione); NF-kB SN50 (AAVALLPAVLLALLAPVQRKRQKLMP); Oridonin; Parthenolide; PPM-18 (2-Benzoylamino-1,4-naphthoquinone); RoI 06-9920; Sulfasalazine; TIRAP Inhibitor Peptide (RQIKIWFNRRMKWKKLQLRDAAPGGAIVS); Withaferin A; Wogonin; BAY 11-7082 ((E)3-[(4-Methylphenyl)sulfonyl]-2-propenenitrile); BAY 11-7085 ((E)3-[(4-t-Butylphenyl)sulfonyl]-2-propenenitrile); (E)-Capsaicin; Aurothiomalate (ATM or AuTM); Evodiamine; Hypoestoxide; IKK Inhibitor m (BMS-345541); IKK Inhibitor VII; IKK Inhibitor X; IKK Inhibitor II; IKK-2 Inhibitor IV; IKK-2 Inhibitor V; IKK-2 Inhibitor VI; IKK-2 Inhibitor (SC-514); IkB Kinase Inhibitor Peptide; IKK-3 Inhibitor IX; ARRY-797 (Array BioPharma); SB-220025 (5-(2-Amino-4-pyrimidinyl)-4-(4-fluorophenyl)-1-(4-piperidinlyl)imidazole); SB-239063 (trans-4-[4-(4-Fluorophenyl)-5-(2-methoxy-4-pyrimidinyl)-1H-imidazol-1-yl]cyclohexanol); SB-202190 (4-(4-Fluorophenyl)-2-(4-hydroxyphenyl)-5-(4-pyridyl)1H-imidazole); JX-401 (-[2-Methoxy-4-(methylthio)benzoyl]-4-(phenylmethyl)piperidine); PD-169316 (4-(4-Fluorophenyl)-2-(4-nitrophenyl)-5-(4-pyridyl)-1H-imidazole); SKF-86002 (6-(4-Fluorophenyl)-2,3-dihydro-5-(4-pyridinyl)imidazo[2,1-b]thiazole dihydrochloride); SB-200646 (N-(I-Methyl-1H-indol-5-yl)-N′-3-pyridinylurea); CMPD-I (2′-Fluoro-N-(4-hydroxyphenyl)-[1,1′-biphenyl]-4-butanamide); EO-1428 ((2-Methylphenyl)-[4-[(2-amino-4-bromophenyl)amino]-2-chlorophenyl]methanone); SB-253080 (4-[5-(4-Fluorophenyl)-2-[4-(methylsulfonyl)phenyl]-1H-imidazol-4-yl]pyridine); SD-169 (1H-Indole-5-carboxamide); SB-203580 (4-(4-Fluorophenyl)-2-(4-methylsulfinyl phenyl)-5-(4-pyridyl)1H-imidazole); TZP-101 (Tranzyme Pharma); TZP-102 (Tranzyme Pharma); GHRP-6 (growth hormone-releasing peptide-6); GHRP-2 (growth hormone-releasing peptide-2); EX-1314 (Elixir Pharmaceuticals); MK-677 (Merck); L-692,429 (Butanamide, 3-amino-3-methyl-N-(2,3,4,5-tetrahydro-2-oxo-1-((2′-(1H-tetrazol-5-yl)(1,1′-biphenyl)-4-yl)methyl)-1H-1-benzazepin-3-yl)-, (R)-); EP1 572 (Aib-DTrp-DgTrp-CHO); diltiazem; metabolites of diltiazem; BRE (Brain and Reproductive organ-Expressed protein); verapamil; nimodipine; diltiazem; omega-conotoxin; GVIA; amlodipine; felodipine; lacidipine; mibefradil; NPPB (5-Nitro-2-(3-phenylpropylamino)benzoic Acid); flunarizine; erythropoietin; piperine; hemin; brazilin; z-V AD-FMK (Benzyloxycarbonyl-Val-Ala-Asp(OMe)-fluoromethylketone); z-LEHD-FMK (benzyloxycarbonyl-Leu-Glu(OMe)-His-Asp(OMe)-fluoromethylketone); B-D-FMK (boc-aspartyl(Ome)-fluoromethylketone); Ac-LEHD-CHO (N-acetyl-Leu-Glu-His-Asp-CHO); Ac-IETD-CHO (N-acetyl-Ile-Glu-Thr-Asp-CHO); z-IETD-FMK (benzyloxycarbonyl-Ile-Glu(OMe)-Thr-Asp(OMe)-fluoromethyl ketone); FAM-LEHD-FMK (benzyloxycarbonyl Leu-Glu-His-Asp-fluoromethyl ketone); FAM-LETD-FMK (benzyloxycarbonyl Leu-Glu-Thr-Asp-iluoromethyl ketone); Q-VD-OPH (Quinoline-Val-Asp-CH2-O-Ph); XIAP; cIAP-1; cIAP-2; ML-IAP; ILP-2; NAIP; Survivin; Bruce; IAPL-3; fortilin; leupeptine; PD-150606 (3-(4-Iodophenyl)-2-mercapto-(Z)-2-propenoic acid); MDL-28170 (Z-Val-Phe-CHO); calpeptin; acetyl-calpastatin; MG 132 (N-t(phenylmethoxy)carbonyl]-L-leucyl-N-[(1S)-1-formyl-3-methylbutyl]-leucinamide); MYODUR; BN 82270 (Ipsen); BN 2204 (Ipsen); AHLi-11 (Quark Pharmaceuticals), an mdm2 protein, pifithrin-α (1-(4-Methylphenyl)-2-(4,5,6,7-tetrahydro-2-imino-3(2H)-benzothiazolyl)ethanone); trans-stilbene; cis-stilbene; resveratrol; piceatannol; rhapontin; deoxyrhapontin; butein; chalcon; isoliquirtigen; butein; 4,2′,4′-trihydroxychalcone; 3,4,2′,4′,6′-pentahydroxychalcone; flavone; morin; fisetin; luteolin; quercetin; kaempferol; apigenin; gossypetin; myricetin; 6-hydroxyapigenin; 5-hydroxyflavone; 5,7,3′,4′,5′-pentahydroxyflavone; 3,7,3′,4′,5′-pentahydroxyflavone; 3,6,3′,4′-tetrahydroxyflavone; 7,3′,4′,5′-tetrahydroxyflavone; 3,6,2′,4′-tetrahydroxyflavone; 7,4′-dihydroxyflavone; 7,8,3′,4′-tetrahydroxyflavone; 3,6,2′,3′-tetrahydroxyflavone; 4′-hydroxyflavone; 5-hydroxyflavone; 5,4′-dihydroxyflavone; 5,7-dihydroxyflavone; daidzein; genistein; naringenin; flavanone; 3,5,7,3′,4′-pentahydroxyflavanone; pelargonidin chloride; cyanidin chloride; delphinidin chloride; (−)-epicatechin (Hydroxy Sites: 3,5,7,3′,4′); (−)-catechin (Hydroxy Sites: 3,5,7,3′,4′); (−)-gallocatechin (Hydroxy Sites: 3,5,7,3′,4′,5′) (+)-catechin (Hydroxy Sites: 3,5,7,3′,4′); (+)-epicatechin (Hydroxy Sites: 3,5,7,3′,4′); Hinokitiol (b-Thujaplicin; 2-hydroxy-4-isopropyl-2,4,6-cycloheptatrien-1-one); L-(+)-Ergothioneine ((S)-a-Carboxy-2,3-dihydro-N,N,N-trimethyl-2-thioxo-1H-imidazole4-ethanaminium inner salt); Caffeic Acid Phenyl Ester; MCI-186 (3-Methyl-1-phenyl-2-pyrazolin-5-one); HBED (N,N′-Di-(2-hydroxybenzyl)ethylenediamine-N,N′-diacetic acid*H₂O); Ambroxol (trans-4-(2-Amino-3,5-dibromobenzylamino)cyclohexane-HCl; and U-83836E ((−)-2-((4-(2,6-di-1-Pyrrolidinyl-4-pyrimidinyl)-1-piperzainyl)methyl)-3,4-dihydro-2,5,7,8-tetramethyl-2H-1-benzopyran-6-ol*2HCl); β-1′-5-methyl-nicotinamide-2′-deoxyribose; β-D-r-5-methyl-nico-tinamide-2′-deoxyribofuranoside; β-1′-4,5-dimethyl-nicotinamide-2′-de-oxyribose; β-D-1 ‘-4,5-dimethyl-nicotinamide-2’-deoxyribofuranoside; 1-Naphthyl PP1 (1-(1,1-Dimethylethyl)-3-(1-naphthalenyl)-1H-pyrazolo[3, 4-d]pyrimidin-4-amine); Lavendustin A (5-[[(2,5-Dihydroxyphenyl)methyl][(2-hydroxyphenyl)methyl]amino]-2-hydroxybenzoic acid); MNS (3,4-Methylenedioxy-b-nitrostyrene), PP1 (1-(1,1-Dimethylethyl)-1-(4-methylphenyl)-1H-pyrazolo[3, 4-d]pyπmidin-4-amine), PP2 (3-(4-chlorophenyl) 1-(1, 1-dimethylethyl)-1H-pyrazolo[3,4-d]pyπmidin-4-amine), KX-004 (Kinex), KX-005 (Kinex), KX-136 (Kinex), KX-174 (Kinex), KX-141 (Kinex), KX2-328 (Kinex), KX-306 (Kinex), KX-329 (Kinex), KX2-391 (Kinex), KX2-377 (Kinex), ZD4190 (Astra Zeneca, N-(4-bromo-2-fluorophenyl)-6-methoxy-7-(2-(1H-1,2,3-triazol-1-yl)ethoxy)quinazolin-4-amine), AP22408 (Ariad Pharmaceuticals), AP23236 (Ariad Pharmaceuticals), AP23451 (Ariad Pharmaceuticals), AP23464 (Ariad Pharmaceuticals), AZD0530 (Astra Zeneca), AZM475271 (M475271, Astra Zeneca), Dasatmib (N-(2-chloro-6-methylphneyl)-2-(6-(4-(2-hydroxyethyl)-piperazin-1-yl)-2-methylpynmidin-4-ylammo) thiazole-5-carboxamide), GN963 (trans-4-(6,7-dimethoxyqmnoxalm-2ylamino)cyclohexanol sulfate); Bosutimb (4-((2,4-dichloro-5-methoxyphenyl)ammo)-6-methoxy-7-(3-(4-methyl-1-piperazinyl)propoxy)-3-quinolinecarbonitrile), or combinations thereof. In some embodiments, a cargo moiety (C) or a therapeutic moiety (T) includes topoisomerase inhibitors (e.g., camptothecin, topotecan), and hypoxia-activated anthraquinone AQ4N; alkylating agents (e.g., temozolomide), agents involved in DNA repair pathways such as poly(ADP ribose)polymerase inhibitors (e.g., AG14361), agents that target Ras family proteins, agents that target epidermal growth factor receptors and associated kinases (e.g., including vandetanib [ZD6474], cetuximab and gefitinib), cyclooxygenase-2 (celecoxib), and derivatives thereof. In some embodiments, T is a topoisomerase inhibitors (e.g., camptothecin, topotecan), and hypoxia-activated anthraquinone AQ4N; alkylating agents (e.g., temozolomide), agents involved in DNA repair pathways such as poly(ADP ribose)polymerase inhibitors (e.g., AG14361), agents that target Ras family proteins, agents that target epidermal growth factor receptors and associated kinases (e.g., including vandetanib [ZD6474], cetuximab and gefitinib), cyclooxygenase-2 (celecoxib), and DM1 (mertansine). In some embodiments, C and/or T is a maytansinoid. In some embodiments, C and/or T is DM1 (mertansine). In some embodiments, C and/or T is not an auristatin or a derivative thereof. In some embodiments, C and/or T is not MMAE or a derivative thereof. In some embodiments, T is a radiosensitizing agent. In some embodiments, the radiosensitizing agent is not an auristatin, including MMAE. In some embodiments, T is a maytansinoid. In some embodiments, T is mertansine.

In some embodiments, a MTS molecule as disclosed herein further comprises a lipid, L. In some embodiments, the MTS comprises a lipid L, A is a peptide with a sequence comprising 5 to 9 consecutive acidic ammo acids, wherein the amino acids are selected from aspartates and glutamates, B is a peptide with a sequence comprising 5 to 20 consecutive basic amino acids, X and/or Y is a linker, and n is an integer between 1 and 20, and wherein L is bound to an MTS as disclosed herein by a bond with a B.

In some embodiments, the lipid entraps a hydrophobic molecule. In some embodiments, the lipid entraps at least one agent selected from the group consisting of a therapeutic moiety and an imaging moiety.

In some embodiments, the lipid is PEGylated. In some embodiments, the lipid is PEG(2K)-phosphatidylethanolamine.

Disclosed herein, in certain embodiments, is a MTS molecule with increased in vivo circulation. In some embodiments, a MTS molecule disclosed herein has the formula according to one of the following:

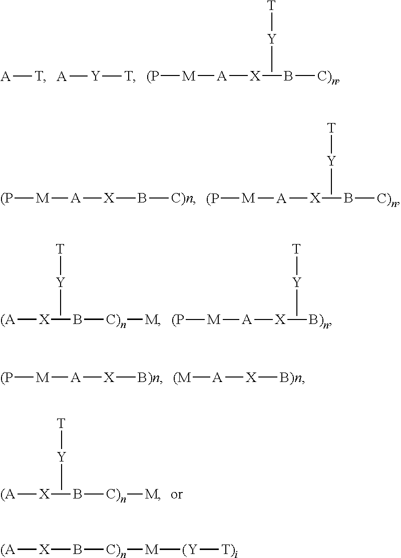

wherein C is a cargo moiety; T is a compound; A is a peptide with a sequence comprising 5 to 9 consecutive acidic ammo acids, wherein the amino acids are selected from aspartates and glutamates, B is a peptide with a sequence comprising 5 to 20 consecutive basic amino acids; X and/or Y is a linker; M is a macromolecular carrier; and n is an integer between 1 and 20. In some embodiments of molecules having features of the invention, n is an integer between 1 and 15. In some embodiments of molecules having features of the invention, n is an integer between 1 and 10. In some embodiments of molecules having features of the invention, n is an integer between 1 and 5. In some embodiments of molecules having features of the invention, n is an integer between 1 and 4. In some embodiments of molecules having features of the invention, n is an integer between 1 and 3. In some embodiments of molecules having features of the invention, n is an integer between 1 and 2. In some embodiments of molecules having features of the invention, n is 1. In some embodiments of molecules having features of the invention, n is an integer of 1, 2, 3, 4, 5, 6, 7, 8, 9, 10, 11, 12, 13, 14, 15, 16, 17, 18, 19, or 20. In some embodiments of molecules having features of the invention, i is an integer between 1 and 20. In some embodiments of molecules having features of the invention, i is an integer between 1 and 10. In some embodiments of molecules having features of the invention, i is an integer between 1 and 5. In some embodiments of molecules having features of the invention, i is an integer between 1 and 4. In some embodiments of molecules having features of the invention, i is an integer between 1 and 3. In some embodiments of molecules having features of the invention, i is an integer between 1 and 2. In some embodiments of molecules having features of the invention, i is 1. In some embodiments of molecules having features of the invention, i is an integer of 1, 2, 3, 4, 5, 6, 7, 8, 9, 10, 11, 12, 13, 14, 15, 16, 17, 18, 19, or 20. In some embodiments, the T-Y pair is attached to either end of B.

In some embodiments, A is an antibody. In some embodiments, A is an anti-ErbB antibody. In some embodiments, A is an anti-HER2 antibody. In some embodiments, the antibody is an anti-EGFR antibody. In some embodiments, A is an antibody selected from the group consisting of cetuximab, trastuzumab, and pertruzumab. In some embodiments, A is cetuximab. In some embodiments, A is trastuzumab. In some embodiments, A is pertruzumab.

The term “macromolecular carrier” indicates an inert molecule that increases (a) plasma half-life and (b) solubility. In some embodiments, a macromolecular carrier decreases uptake of a MTS molecule into cartilage. In some embodiments, a macromolecular carrier decreases uptake of a MTS molecule into joints. In some embodiments, a macromolecular carrier decreases uptake of a MTS molecule into the liver. In some embodiments, a macromolecular carrier decreases uptake of a MTS molecule into kidneys.

In some embodiments, a macromolecular carrier M increases plasma half-life and solubility by reducing glomerular filtration. In some embodiments, a macromolecular carrier increases tumor uptake due to enhanced permeability and retention (EPR) of tumor vasculature.

In some embodiments, M is bound to A. In some embodiments, M is bound to A at the n-terminal poly glutamate. In some embodiments, M is bound to A (or, the n-terminal poly glutamate) by a covalent linkage. In some embodiments, the covalent linkage comprises an ether bond, thioether bond, amine bond, amide bond, carbon-carbon bond, carbon-nitrogen bond, carbon-oxygen bond, or carbon-sulfur bond.

In some embodiments, M is bound to B. In some embodiments, M is bound to B at the c-terminal polyarginine. In some embodiments, M is bound to B (or, the c-terminal polyarginine) by a covalent linkage. In some embodiments, the covalent linkage comprises an ether bond, thioether bond, amine bond, amide bond, carbon-carbon bond, carbon-nitrogen bond, carbon-oxygen bond, or carbon-sulfur bond. In some embodiments, M is bound to A.

In some embodiments, M is selected from a protein, a synthetic or natural polymer, or a dendrimer. In some embodiments, M is a dendrimer, dextran (including for example but not limited to branched glucan/branched glucose molecules composed of chains of varying lengths ranging from 3 to 2000 kDa with molecular weights ranging from 3 kDa to 2,000 kDa, for example but not limited to 20 kDa, 40 kDa, 60 kDa and 100 kDa), a PEG polymer (e.g., PEG 5 kDa, PEG 10 kDa, PEG 12 kDa, PEG 15 kDa, PEG 20 kDa, PEG 40 kDa, PEG 50 kDa or PEG 100 kDa), albumin or fragments thereof, lipid-coated perfluorocarbon droplet or a combination thereof. In some embodiments, M is a PEG polymer.

In some embodiments, the size of the macromolecular carrier is between 50 kDa and 70 kDa. In some embodiments, small amounts of negative charge keep peptides out of the liver while not causing synovial uptake.

In some embodiments, the MTS molecule is conjugated to albumin. In certain instances, albumin is excluded from the glomerular filtrate under normal physiological conditions. In some embodiments, the MTS molecule comprises a reactive group such as maleimide that can form a covalent conjugate with albumin. A MTS molecule comprising albumin results in enhanced accumulation of cleaved MTS molecules in tumors in a cleavage dependent manner. In some embodiments, albumin conjugates have good pharmacokinetic properties but are difficult to work with synthetically.

In some embodiments, the MTS molecule is conjugated to a PEG polymer. In some embodiments, the MTS molecule is conjugated to a PEG 5 kDa polymer. In some embodiments, the MTS molecule is conjugated to a PEG 12 kDa polymer. In some embodiments, 5 kDa PEG conjugates behaved similarly to free peptides. In some embodiments, 12 kDa PEG conjugates had a longer half-life as compared to free peptides. See Example 5 for a detailed analysis of the effects of using a PEG polymer.

In some embodiments, the MTS molecule is conjugated to a dextran. In some embodiments, the MTS molecule is conjugated to a 70 kDa dextran. In some embodiments, dextran conjugates, being a mixture of molecular weights, are difficult to synthesize and purify reproducibly.

In some embodiments, the MTS molecule is conjugated to streptavidin.

In some embodiments, the MTS molecule is conjugated to a fifth generation PAMAM dendrimer.

In some embodiments, a macromolecular carrier is capped. In some embodiments, capping a carrier improves the pharmacokinetics and reduces cytotoxicity of a macromolecular carrier by adding hydrophilicity. In some embodiments, the cap is selected from: Acetyl, succinyl, 3-hydroxypropionyl, 2-sulfobenzoyl, glycidyl, PEG-2, PEG-4, PEG-8 and PEG-12.

In some embodiments, the macromolecular carrier comprises a dendrimer. As used herein, “dendrimer” means a poly-functional (or, poly-branched) molecule. In some embodiments, a dendrimer is a structure in which a central molecule branches repetitively and repetitiously. In some embodiments, the dendrimer is a nanoparticle. In some embodiments, the dendrimer comprises a reactive group such as maleimide that can form a covalent conjugate with albumin. In some embodiments, a dendrimer is conjugated to a MTS molecule via a maleimide linker at the C-terminal end of the MTS molecule. In some embodiments, when the MTS molecule comprises a dendrimer, the cargo and/or compound T is attached directly to D.

In some embodiments, conjugating a MTS molecule to a dendrimer increases plasma half-life as compared to an unconjugated (or, free) MTS molecule. In some embodiments, a MTS molecule conjugated to a dendrimer results in a decrease in acute toxicity as compared to unconjugated MTS molecules. In some embodiments, a MTS molecule conjugated to a dendrimer reduces uptake by synovium, cartilage and kidney as compared to unconjugated MTS molecules.

In some embodiments, a MTS molecule conjugated to a dendrimeric nanoparticle is used to target tumor associated macrophages. In some embodiments, a MTS molecule conjugated to a dendrimeric nanoparticle, wherein the nanoparticle further comprises Ricin A, is used to poison subcutaneous macrophages.

MTS molecules having features of disclosed herein may be synthesized by standard synthetic techniques, such as, for example, solid phase synthesis including solid phase peptide synthesis. (An example of peptide synthesis using Fmoc is given as Example 1 in WO 2005/042034). For example, conventional solid phase methods for synthesizing peptides may start with N-alpha-protected amino acid anhydrides that are prepared in crystallized form or prepared freshly in solution, and are used for successive amino acid addition at the N-terminus. At each residue addition, the growing peptide (on a solid support) is acid treated to remove the N-alpha-protective group, washed several times to remove residual acid and to promote accessibility of the peptide terminus to the reaction medium. The peptide is then reacted with an activated N-protected amino acid symmetrical anhydride, and the solid support is washed. At each residue-addition step, the amino acid addition reaction may be repeated for a total of two or three separate addition reactions, to increase the percent of growing peptide molecules which are reacted. Typically, 1 to 2 reaction cycles are used for the first twelve residue additions, and 2 to 3 reaction cycles for the remaining residues.

After completing the growing peptide chains, the protected peptide resin is treated with a strong acid such as liquid hydrofluoric acid or trifluoroacetic acid to deblock and release the peptides from the support. For preparing an amidated peptide, the resin support used in the synthesis is selected to supply a C-terminal amide, after peptide cleavage from the resin. After removal of the strong acid, the peptide may be extracted into 1M acetic acid solution and lyophilized. The peptide can be isolated by an initial separation by gel filtration, to remove peptide dimers and higher molecular weight polymers, and also to remove undesired salts The partially purified peptide may be further purified by preparative HPLC chromatography, and the purity and identity of the peptide confirmed by amino acid composition analysis, mass spectrometry and by analytical HPLC (e.g., in two different solvent systems).

The invention also provides polynucleotides encoding MTS molecules described herein. The term “polynucleotide” refers to a polymeric form of nucleotides of at least 10 bases in length. The nucleotides can be ribonucleotides, deoxynucleotides, or modified forms of either type of nucleotide. The term includes single and double stranded forms of DNA. The term therefore includes, for example, a recombinant DNA which is incorporated into a vector, e.g., an expression vector; into an autonomously replicating plasmid or virus; or into the genomic DNA of a prokaryote or eukaryote, or which exists as a separate molecule (e.g., a cDNA) independent of other sequences.

These polynucleotides include DNA, cDNA, and RNA sequences which encode MTS molecules having features of the invention, or portions thereof. Peptide portions may be produced by recombinant means, including synthesis by polynucleotides encoding the desired amino acid sequence. Such polynucleotides may also include promoter and other sequences, and may be incorporated into a vector for transfection (which may be stable or transient) in a host cell.

The construction of expression vectors and the expression of genes in transfected cells involves the use of molecular cloning techniques that are well known in the art. See, for example, Sambrook et al., Molecular Cloning—A Laboratory Manual, Cold Spring Harbor Laboratory, Cold Spring Harbor, N.Y., (1989) and Current Protocols in Molecular Biology, F. M. Ausubel et al., eds., (Current Protocols, a joint venture between Greene Publishing Associates, Inc. and John Wiley & Sons, Inc., most recent Supplement). Nucleic acids used to transfect cells with sequences coding for expression of the polypeptide of interest generally will be in the form of an expression vector including expression control sequences operatively linked to a nucleotide sequence coding for expression of the polypeptide. As used herein, “operatively linked” refers to a juxtaposition wherein the components so described are in a relationship permitting them to function in their intended manner. A control sequence operatively linked to a coding sequence is ligated such that expression of the coding sequence is achieved under conditions compatible with the control sequences. “Control sequence” refers to polynucleotide sequences which are necessary to effect the expression of coding and non-coding sequences to which they are ligated. Control sequences generally include promoter, ribosomal binding site, and transcription termination sequence. The term “control sequences” is intended to include, at a minimum, components whose presence can influence expression, and can also include additional components whose presence is advantageous, for example, leader sequences and fusion partner sequences. As used herein, the term “nucleotide sequence coding for expression of a polypeptide refers to a sequence that, upon transcription and translation of mRNA, produces the polypeptide. This can include sequences containing, e.g., introns. As used herein, the term “expression control sequences” refers to nucleic acid sequences that regulate the expression of a nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (i.e., ATG) in front of a protein-encoding gene, splicing signals for introns, maintenance of the correct reading frame of that gene to permit proper translation of the mRNA, and stop codons.

Any suitable method is used to construct expression vectors containing the fluorescent indicator coding sequence and appropriate transcriptional/translational control signals. Any methods which are well known to those skilled in the art can be used to construct expression vectors containing the fluorescent indicator coding sequence and appropriate transcriptional/translational control signals. These methods include in vitro recombinant DNA techniques, synthetic techniques and in vivo recombination/genetic recombination. (See, for example, the techniques described in Maniatis, et al., Molecular Cloning A Laboratory Manual, Cold Spring Harbor Laboratory, N.Y., 1989). Transformation of a host cell with recombinant DNA may be carried out by conventional techniques as are well known to those skilled in the art.

Where the host is prokaryotic, such as E. coli, competent cells which are capable of DNA uptake can be prepared from cells harvested after exponential growth phase and subsequently treated by the CaCl₂ method by procedures well known in the art. Alternatively, MgCl₂ or RbCl can be used. Transformation can also be performed after forming a protoplast of the host cell or by electroporation.

When the host is a eukaryote, such methods of transfection of DNA as calcium phosphate co-precipitates, conventional mechanical procedures such as microinjection, electroporation, insertion of a plasmid encased in liposomes, or virus vectors may be used. Eukaryotic cells can also be cotransfected with DNA sequences encoding the fusion polypeptide of the invention, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein. (Eukagotic Viral Vectors, Cold Spring Harbor Laboratory, Gluzman ed., 1982). Techniques for the isolation and purification of polypeptides of the invention expressed in prokaryotes or eukaryotes may be by any conventional means such as, for example, preparative chromatographic separations and immunological separations such as those involving the use of monoclonal or polyclonal antibodies or antigen.

It will be understood that the compounds of the present invention can be formulated in pharmaceutically and or diagnostically useful compositions. Such pharmaceutical and diagnostically useful compositions may be prepared according to well-known methods. For example, MTS compounds having features of the invention, and having a cargo portion C that is, for example, a therapeutic moiety or a detection moiety, may be combined in admixture with a pharmaceutically acceptable carrier vehicle or a diagnostic buffering agent. Suitable vehicles and agents and their formulation, inclusive of other human proteins, e.g. human serum albumin are described, for example, in Remington's Pharmaceutical Sciences by E. W. Martin and, the techniques described in Maniatis, et al., Molecular Cloning A Laboratory Manual, Cold Spring Harbor Laboratory, N.Y., 1989-2013, which are hereby incorporated by reference. Such compositions will contain an effective amount of the compounds hereof together with a suitable amount of vehicle in order to prepare pharmaceutically acceptable compositions suitable for effective administration. Dosages and dosing regimens may be determined for the indications and compounds by methods known in the art, including determining (e.g., in experimental animals) the effective dose which causes half of those treated to respond to the treatment (ED₅₀) by providing a range of doses to experimental animals or subjects and noting the responses.

### Methods of Use

The MTS molecules find use in a variety of ex vivo applications as described herein and such MTS molecules have been thoroughly described (see, WO 2005/042034, WO/2006/125134, WO2011008992 and WO2011008996; all of which are incorporated herein by reference in their entireties). As such, according to disclosure contained herein, this invention pertains to methods and compositions that find use in treatment, diagnostic, prognostic (e.g., patient prognosis) and characterization (e.g., histologic grade/stage) of neoplasia and neoplasma samples based on the ability of a tumor sample to cleave a MTS molecule of the present invention.

Methods of use and compositions comprising MTS molecules are disclosed. Molecules having features of the invention include peptide portions linked by a cleavable linker portion which may be a peptide. The inventors have found that these MTS molecules can find use in treatment, diagnostic, detection, screening, prognosis (e.g., patient prognosis) and characterization (e.g., histologic grade/stage) assays.

According to the present invention, such methods are based in part on cleavage of the MTS molecule and detection of that cleavage event. The presence of one or more proteases in a sample from a subject can be detected ex vivo based on cleavage of the peptide. Such cleavage is detected by detecting a change in a detectable label (detectable moiety) that is part of the MTS peptide. In some embodiments, the MTS molecule contains a detectable moieties which provide for an indication of a cleavage event. In some embodiments, cleavage could be detected by size changes in the length of the peptide (e.g., gel electrophoresis, size exclusion, column chromatography, immunofluorescence, etc.) or other biochemical and physical changes that occur to the MTS molecule. In some embodiments, the MTS molecule comprises a label which facilitates cleavage detection. In some embodiments, cleavage could be detected using a FRET-based pair (a reporter dye and an acceptor dye that are involved in fluorescence resonance energy transfer known as FRET), where a change in fluorescence is indicative of a cleavage event. See, for examples, Examples 1-3. Methods for detecting and monitoring cleavage of proteins are well known and any such methods could be employed in detecting cleavage of the MTS molecules of the invention.

In some embodiments, the invention provides an ex vivo method for detecting the presence of one or more protease activities in a neoplasia sample comprising a) combining ex vivo said sample from a subject with a molecule of the structure according to one of the following:

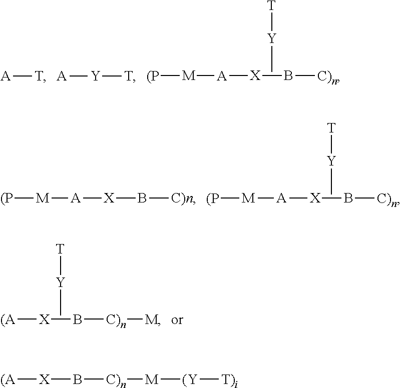

wherein cleavage of said molecule is indicative of the presence of protease activity and wherein P is an optional pre-targeting moiety; M is an optional macromolecular carrier; A is peptide portion with a sequence comprising about 2 to about 20 acidic amino acids, which when linked with portion B is effective to inhibit or prevent cellular uptake of portion B; B is a peptide portion of about 5 to about 20 basic amino acid residues, which is suitable for cellular uptake; X is a first cleavable linker of about 2 to about 100 atoms, or about 3 to about 30 atoms, joining A with B; Y is a second cleavable linker of about 2 to about 100 atoms, or about 3 to about 30 atoms, joining A-X-B with T; T is a compound for delivery to a target, including for example a therapeutic compound (also referred to herein as a “warhead”); and n is an integer between 1 and 20; and C is a detectable moiety and b) detecting cleavage of said molecule by detecting a change in said detectable moiety C, wherein said change in C is indicative of cleavage and said cleavage is indicative of the presence of one or more protease activities in said neoplasia. In some embodiments, X is a cleavable linker of 2 or more atoms, 10 or more, 50 or more atoms, 100 or more atoms, 200 or more atoms, 300 or more atoms, 400 or more atoms or 500 or more atoms. In some embodiments, X is a cleavable linker cleavable by a matrix metalloproteinase (MMP) or a reactive oxygen species. In some embodiments, the screening is small scale, involving screening of 1, 5, 10, 20 or 30 samples. In some embodiments, Y is a cleavable linker of 2 or more atoms, 10 or more, 50 or more atoms, 100 or more atoms, 200 or more atoms, 300 or more atoms, 400 or more atoms or 500 or more atoms. In some embodiments, Y is a cleavable linker cleavable by a matrix metalloproteinase (MMP) or a reactive oxygen species. In some embodiments, one protease activity can be detected. In some embodiments, 2, 3, 4, 5, 6, 7, 8, 9 or 10 protease activities can be detected. In some embodiments, one or more protease activities can be detected. In some embodiments, said portion A has between about 5 to about 9 acidic amino acid residues, and said portion B has between about 9 to about 16 basic amino acid residues.

In some embodiments, A is an antibody. In some embodiments, A is an anti-ErbB antibody. In some embodiments, A is an anti-HER2 antibody. In some embodiments, the antibody is an anti-EGFR antibody. In some embodiments, A is an antibody selected from the group consisting of cetuximab, trastuzumab, and pertruzumab. In some embodiments, A is cetuximab. In some embodiments, A is trastuzumab. In some embodiments, A is pertruzumab.

In some embodiments, the invention provides an ex vivo method of screening for the presence of one or more protease activities in a neoplasia sample comprising combining ex vivo said neoplasia sample from a subject with a molecule of the structure according to one of the following:

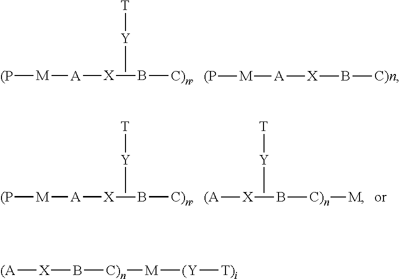

wherein cleavage of said molecule is indicative of the presence of protease activity and wherein P is an optional pre-targeting moiety; M is an optional macromolecular carrier; A is peptide portion with a sequence comprising about 2 to about 20 acidic amino acids, which when linked with portion B is effective to inhibit or prevent cellular uptake of portion B; B is a peptide portion of about 5 to about 20 basic amino acid residues, which is suitable for cellular uptake; X is a first cleavable linker of about 2 to about 100 atoms, or about 3 to about 30 atoms, joining A with B; Y is a second cleavable linker of about 2 to about 100 atoms, or about 3 to about 30 atoms, joining A-X-B with T; T is a compound for delivery to a target, including for example a therapeutic compound (also referred to herein as a “warhead”); and n is an integer between 1 and 20; and C is a detectable moiety; and b) detecting cleavage of said molecule by detecting a change in said detectable moiety C, wherein said change in C is indicative of cleavage and said cleavage is indicative of the presence of one or more protease activities in said neoplasia. In some embodiments the MTS molecules can be used in screening assays to determine how many proteases and/or which proteases are expressed by a sample. In some embodiments, X is a cleavable linker of 2 or more atoms, 10 or more, 50 or more atoms, 100 or more atoms, 200 or more atoms, 300 or more atoms, 400 or more atoms or 500 or more atoms. In some embodiments, X is a cleavable linker cleavable by a matrix metalloproteinase (MMP) or a reactive oxygen species. In some embodiments, Y is a cleavable linker of 2 or more atoms, 10 or more, 50 or more atoms, 100 or more atoms, 200 or more atoms, 300 or more atoms, 400 or more atoms or 500 or more atoms. In some embodiments, Y is a cleavable linker cleavable by a matrix metalloproteinase (MMP) or a reactive oxygen species. In some embodiments, the screening is small scale, involving screening of 1, 5, 10, 20 or 30 samples. In some embodiments, screening is large scale, and involves screening of 100, 500, 1000, 10000, 100 000, 500000 or more samples. In some embodiments, samples are screened for 1, 2, 3, 4, 5, 6, 7, 8, 9, 10 or more protease activities using MTS molecules of the invention. In some embodiments, screening information can be employed to develop data bases and incorporated with other bioinformatic information in order to develop hydrogen peroxide profiles of samples. In some embodiments, said portion A has between about 5 to about 9 acidic amino acid residues, and said portion B has between about 9 to about 16 basic amino acid residues.

In some embodiments, A is an antibody. In some embodiments, A is an anti-ErbB antibody. In some embodiments, A is an anti-HER2 antibody. In some embodiments, the antibody is an anti-EGFR antibody. In some embodiments, A is an antibody selected from the group consisting of cetuximab, trastuzumab, and pertruzumab. In some embodiments, A is cetuximab. In some embodiments, A is trastuzumab. In some embodiments, A is pertruzumab

In some embodiments, the invention provides an ex vivo method of determining the protease profile of a neoplasia sample, comprising a) combining said sample from a subject with a molecule of the structure according to one of the following:

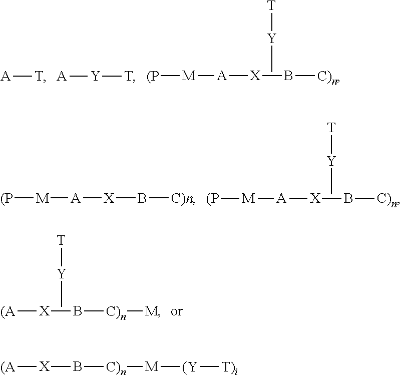

wherein cleavage of said molecule is indicative of the presence of protease activity and wherein P is an optional pre-targeting moiety; M is an optional macromolecular carrier; A is peptide portion with a sequence comprising about 2 to about 20 acidic amino acids, which when linked with portion B is effective to inhibit or prevent cellular uptake of portion B; B is a peptide portion of about 5 to about 20 basic amino acid residues, which is suitable for cellular uptake; X is a first cleavable linker of about 2 to about 100 atoms, or about 3 to about 30 atoms, joining A with B; Y is a second cleavable linker of about 2 to about 100 atoms, or about 3 to about 30 atoms, joining A-X-B with T; T is a compound for delivery to a target, including for example a therapeutic compound (also referred to herein as a “warhead”); and n is an integer between 1 and 20; and C is a detectable moiety; and b) detecting cleavage of said molecule by detecting a change in said detectable moiety C, wherein said change in C is indicative of cleavage and said cleavage is indicative of the presence of one or more protease activities in said neoplasia. In some embodiments the MTS molecules can be used in screening assays to determine how many proteases and/or which proteases are expressed by a sample. In some embodiments, X is a cleavable linker of 2 or more atoms, 10 or more, 50 or more atoms, 100 or more atoms, 200 or more atoms, 300 or more atoms, 400 or more atoms or 500 or more atoms. In some embodiments, X is a cleavable linker cleavable by a matrix metalloproteinase (MMP) or a reactive oxygen species. In some embodiments, Y is a cleavable linker of 2 or more atoms, 10 or more, 50 or more atoms, 100 or more atoms, 200 or more atoms, 300 or more atoms, 400 or more atoms or 500 or more atoms. In some embodiments, Y is a cleavable linker cleavable by a matrix metalloproteinase (MMP) or a reactive oxygen species. In some embodiments, the MTS molecules are employed to develop a protease profile for one or more neoplasia samples. Hydrogen peroxide profiles can be employed to develop databases and can be incorporated with other information, including for example bioinformatic information, in order to develop hydrogen peroxide profiles of disease samples and for hydrogen peroxide profiles for patients with diseases. Diseases contemplated by the methods of the present invention include inflammatory diseases, neurodegenerative diseases, cardiovascular diseases, diabetes and neoplasia. In some embodiments, said portion A has between about 5 to about 9 acidic amino acid residues, and said portion B has between about 9 to about 16 basic amino acid residues. In some embodiments of molecules having features of the invention, n is an integer between 1 and 20. In some embodiments of molecules having features of the invention, n is an integer between 1 and 15. In some embodiments of molecules having features of the invention, n is an integer between 1 and 10. In some embodiments of molecules having features of the invention, n is an integer between 1 and 5. In some embodiments of molecules having features of the invention, n is an integer between 1 and 4. In some embodiments of molecules having features of the invention, n is an integer between 1 and 3. In some embodiments of molecules having features of the invention, n is an integer between 1 and 2. In some embodiments of molecules having features of the invention, n is 1. In some embodiments of molecules having features of the invention, n is an integer of 1, 2, 3, 4, 5, 6, 7, 8, 9, 10, 11, 12, 13, 14, 15, 16, 17, 18, 19, or 20. In some embodiments of molecules having features of the invention, i is an integer between 1 and 20. In some embodiments of molecules having features of the invention, i is an integer between 1 and 10. In some embodiments of molecules having features of the invention, i is an integer between 1 and 5. In some embodiments of molecules having features of the invention, i is an integer between 1 and 4. In some embodiments of molecules having features of the invention, i is an integer between 1 and 3. In some embodiments of molecules having features of the invention, i is an integer between 1 and 2. In some embodiments of molecules having features of the invention, i is 1. In some embodiments of molecules having features of the invention, i is an integer of 1, 2, 3, 4, 5, 6, 7, 8, 9, 10, 11, 12, 13, 14, 15, 16, 17, 18, 19, or 20. In some embodiments, the T-Y pair is attached to either end of B.

In some embodiments, A is an antibody. In some embodiments, A is an anti-ErbB antibody. In some embodiments, A is an anti-HER2 antibody. In some embodiments, the antibody is an anti-EGFR antibody. In some embodiments, A is an antibody selected from the group consisting of cetuximab, trastuzumab, and pertruzumab. In some embodiments, A is cetuximab. In some embodiments, A is trastuzumab. In some embodiments, A is pertruzumab.

In some embodiments, the invention provides an ex vivo method of determining a treatment regimen based on the protease profile of a neoplasia sample, comprising a) combining ex vivo said neoplasia sample from a subject with a molecule of the structure according to one of the following:

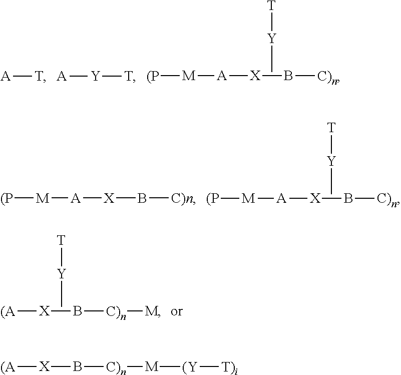

wherein cleavage of said molecule is indicative of the presence of protease activity and wherein P is an optional pre-targeting moiety; M is an optional macromolecular carrier; A is peptide portion with a sequence comprising about 2 to about 20 acidic amino acids, which when linked with portion B is effective to inhibit or prevent cellular uptake of portion B; B is a peptide portion of about 5 to about 20 basic amino acid residues, which is suitable for cellular uptake; X is a first cleavable linker of about 2 to about 100 atoms, or about 3 to about 30 atoms, joining A with B; Y is a second cleavable linker of about 2 to about 100 atoms, or about 3 to about 30 atoms, joining A-X-B with T; T is a compound for delivery to a target, including for example a therapeutic compound (also referred to herein as a “warhead”); and n is an integer between 1 and 20; and C is a detectable moiety; and b) detecting cleavage of said molecule by detecting a change in detectable moiety C, wherein said change in C is indicative of cleavage and said cleavage is indicative of the presence of one or more protease activities and wherein the presence and/or absence of one or more protease activities allows for determining a medical treatment regimen. In some embodiments, X is a cleavable linker of 2 or more atoms, 10 or more, 50 or more atoms, 100 or more atoms, 200 or more atoms, 300 or more atoms, 400 or more atoms or 500 or more atoms. In some embodiments, X is a cleavable linker cleavable by a matrix metalloproteinase (MMP) or a reactive oxygen species. In some embodiments, Y is a cleavable linker of 2 or more atoms, 10 or more, 50 or more atoms, 100 or more atoms, 200 or more atoms, 300 or more atoms, 400 or more atoms or 500 or more atoms. In some embodiments, Y is a cleavable linker cleavable by a matrix metalloproteinase (MMP) or a reactive oxygen species. In some embodiments, the MTS molecules are employed to determine a treatment regimen. Matrix metalloproteinase (MMP), hydrogen peroxide or other cleavage information and/or profiles can be employed to develop databases and can be incorporated with other information, for example bioinformatic information, in order to develop cleavage profiles of samples. In some embodiments, such information can be combined with information regarding treatment and surgical options know to those of skill in the medical arts in order to determine and develop personalize treatment regimens for individual subjects. In some embodiments, the medical regimen is a surgical regimen. After detecting the presence or absence of one or more proteases based on MTS molecule cleavage, a determination of the usefulness of an MTS molecule in surgical procedures can be determined. Detection of cleavage of the MTS molecule would be indicative of the presence of one or more proteases and such information would allow for a determination of usefulness of the peptide in a surgical procedure in order to detect tumor borders and assist with surgical removal as previously described (See, e.g., see, WO 2005/042034, WO/2006/125134, WO2011008992 and WO2011008996). Non-detection of cleavage of the MTS molecule would be indicative of the absence of a protease and the non-usefulness of the peptide in a surgical procedure. In some embodiments, said portion A has between about 5 to about 9 acidic amino acid residues, and said portion B has between about 9 to about 16 basic amino acid residues. In some embodiments of molecules having features of the invention, n is an integer between 1 and 20. In some embodiments of molecules having features of the invention, n is an integer between 1 and 15. In some embodiments of molecules having features of the invention, n is an integer between 1 and 10. In some embodiments of molecules having features of the invention, n is an integer between 1 and 5. In some embodiments of molecules having features of the invention, n is an integer between 1 and 4. In some embodiments of molecules having features of the invention, n is an integer between 1 and 3. In some embodiments of molecules having features of the invention, n is an integer between 1 and 2. In some embodiments of molecules having features of the invention, n is 1. In some embodiments of molecules having features of the invention, n is an integer of 1, 2, 3, 4, 5, 6, 7, 8, 9, 10, 11, 12, 13, 14, 15, 16, 17, 18, 19, or 20. In some embodiments of molecules having features of the invention, i is an integer between 1 and 20. In some embodiments of molecules having features of the invention, i is an integer between 1 and 10. In some embodiments of molecules having features of the invention, i is an integer between 1 and 5. In some embodiments of molecules having features of the invention, i is an integer between 1 and 4. In some embodiments of molecules having features of the invention, i is an integer between 1 and 3. In some embodiments of molecules having features of the invention, i is an integer between 1 and 2. In some embodiments of molecules having features of the invention, i is 1. In some embodiments of molecules having features of the invention, i is an integer of 1, 2, 3, 4, 5, 6, 7, 8, 9, 10, 11, 12, 13, 14, 15, 16, 17, 18, 19, or 20. In some embodiments, the T-Y pair is attached to either end of B.

In some embodiments, A is an antibody. In some embodiments, A is an anti-ErbB antibody. In some embodiments, A is an anti-HER2 antibody. In some embodiments, the antibody is an anti-EGFR antibody. In some embodiments, A is an antibody selected from the group consisting of cetuximab, trastuzumab, and pertruzumab. In some embodiments, A is cetuximab. In some embodiments, A is trastuzumab. In some embodiments, A is pertruzumab.

In some embodiments, the invention provides an ex vivo method of characterizing a neoplasia based on the protease profile of said neoplasia, comprising a) combining a sample of said neoplasia from a subject with a molecule of the structure according to one of the following:

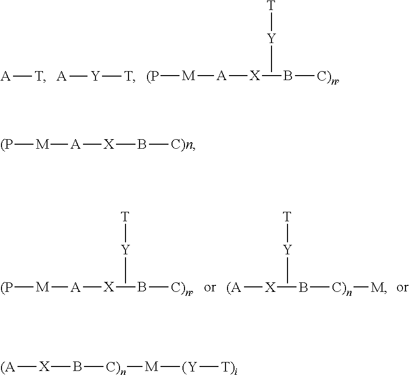

wherein cleavage of said molecule is indicative of the presence of protease activity and wherein P is an optional pre-targeting moiety; M is an optional macromolecular carrier; A is peptide portion with a sequence comprising about 2 to about 20 acidic amino acids, which when linked with portion B is effective to inhibit or prevent cellular uptake of portion B; B is a peptide portion of about 5 to about 20 basic amino acid residues, which is suitable for cellular uptake; X is a first cleavable linker of about 2 to about 100 atoms, or about 3 to about 30 atoms, joining A with B; Y is a second cleavable linker of about 2 to about 100 atoms, or about 3 to about 30 atoms, joining A-X-B with T; T is a compound for delivery to a target, including for example a therapeutic compound (also referred to herein as a “warhead”); and n is an integer between 1 and 20; and C is a detectable moiety; and b) detecting cleavage of said molecule by detecting a change in detectable moiety C, wherein said change in C is indicative of cleavage and said cleavage is indicative of the presence of one or more protease activities and wherein the presence and/or absence of one or more protease activities allows for determining a medical treatment regimen. In some embodiments, X is a cleavable linker of 2 or more atoms, 10 or more, 50 or more atoms, 100 or more atoms, 200 or more atoms, 300 or more atoms, 400 or more atoms or 500 or more atoms. In some embodiments, X is a cleavable linker cleavable by a matrix metalloproteinase (MMP) or a reactive oxygen species. In some embodiments, Y is a cleavable linker of 2 or more atoms, 10 or more, 50 or more atoms, 100 or more atoms, 200 or more atoms, 300 or more atoms, 400 or more atoms or 500 or more atoms. In some embodiments, Y is a cleavable linker cleavable by a matrix metalloproteinase (MMP) or a reactive oxygen species. In some embodiments, the neoplasia is characterized based on histology, stage, grade, location, type, or any of a variety of characteristics known to those skilled in the medical arts. In some embodiments, the protease profile is correlated with histology, stage, grade, location, type, or any of a variety of characteristics known to those skilled in the medical arts in order to characterize the neoplasia. In some embodiments, the presence of the protease activity is indicative of neoplasia. In some embodiments, the presence of the protease activity is indicative of metastasis. In some embodiments, said portion A has between about 5 to about 9 acidic amino acid residues, and said portion B has between about 9 to about 16 basic amino acid residues. In some embodiments of molecules having features of the invention, n is an integer between 1 and 20. In some embodiments of molecules having features of the invention, n is an integer between 1 and 15. In some embodiments of molecules having features of the invention, n is an integer between 1 and 10. In some embodiments of molecules having features of the invention, n is an integer between 1 and 5. In some embodiments of molecules having features of the invention, n is an integer between 1 and 4. In some embodiments of molecules having features of the invention, n is an integer between 1 and 3. In some embodiments of molecules having features of the invention, n is an integer between 1 and 2. In some embodiments of molecules having features of the invention, n is 1. In some embodiments of molecules having features of the invention, n is an integer of 1, 2, 3, 4, 5, 6, 7, 8, 9, 10, 11, 12, 13, 14, 15, 16, 17, 18, 19, or 20. In some embodiments of molecules having features of the invention, i is an integer between 1 and 20. In some embodiments of molecules having features of the invention, i is an integer between 1 and 10. In some embodiments of molecules having features of the invention, i is an integer between 1 and 5. In some embodiments of molecules having features of the invention, i is an integer between 1 and 4. In some embodiments of molecules having features of the invention, i is an integer between 1 and 3. In some embodiments of molecules having features of the invention, i is an integer between 1 and 2. In some embodiments of molecules having features of the invention, i is 1. In some embodiments of molecules having features of the invention, i is an integer of 1, 2, 3, 4, 5, 6, 7, 8, 9, 10, 11, 12, 13, 14, 15, 16, 17, 18, 19, or 20. In some embodiments, the T-Y pair is attached to either end of B.

In some embodiments, A is an antibody. In some embodiments, A is an anti-ErbB antibody. In some embodiments, A is an anti-HER2 antibody. In some embodiments, the antibody is an anti-EGFR antibody. In some embodiments, A is an antibody selected from the group consisting of cetuximab, trastuzumab, and pertruzumab. In some embodiments, A is cetuximab. In some embodiments, A is trastuzumab. In some embodiments, A is pertruzumab.

In some embodiments, the present invention provides a diagnostic composition for use in the methods of any of the preceding claims comprising: a molecule of the structure according to one of the following:

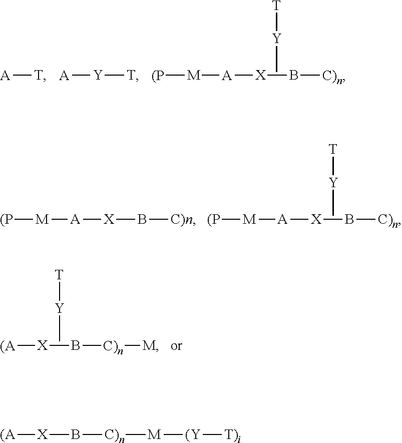

wherein cleavage of said molecule is indicative of the presence of protease activity and wherein P is an optional pre-targeting moiety; M is an optional macromolecular carrier; A is peptide portion with a sequence comprising about 2 to about 20 acidic amino acids, which when linked with portion B is effective to inhibit or prevent cellular uptake of portion B; B is a peptide portion of about 5 to about 20 basic amino acid residues, which is suitable for cellular uptake; X is a first cleavable linker of about 2 to about 100 atoms, or about 3 to about 30 atoms, joining A with B and is cleavable under physiological conditions; Y is a second cleavable linker of about 2 to about 100 atoms, or about 3 to about 30 atoms, joining A-X-B with T and is cleavable under physiological conditions; T is a compound for delivery to a target, including for example a therapeutic compound (also referred to herein as a “warhead”); and n is an integer between 1 and 20; and C is a detectable moiety; and b) detecting cleavage of said molecule by detecting a change in detectable moiety C, wherein said change in C is indicative of cleavage and said cleavage is indicative of the presence of one or more protease activities and wherein the presence and/or absence of one or more protease activities allows for determining a medical treatment regimen. In some embodiments, X is a cleavable linker of 2 or more atoms, 10 or more, 50 or more atoms, 100 or more atoms, 200 or more atoms, 300 or more atoms, 400 or more atoms or 500 or more atoms. In some embodiments, X is a cleavable linker cleavable by a matrix metalloproteinase (MMP) or a reactive oxygen species. In some embodiments, Y is a cleavable linker of 2 or more atoms, 10 or more, 50 or more atoms, 100 or more atoms, 200 or more atoms, 300 or more atoms, 400 or more atoms or 500 or more atoms. In some embodiments, Y is a cleavable linker cleavable by a matrix metalloproteinase (MMP) or a reactive oxygen species. In some embodiments of the diagnostic composition, said portion A has between about 5 to about 9 acidic amino acid residues, and said portion B has between about 9 to about 16 basic amino acid residues. In some embodiments of molecules having features of the invention, n is an integer between 1 and 20. In some embodiments of molecules having features of the invention, n is an integer between 1 and 15. In some embodiments of molecules having features of the invention, n is an integer between 1 and 10. In some embodiments of molecules having features of the invention, n is an integer between 1 and 5. In some embodiments of molecules having features of the invention, n is an integer between 1 and 4. In some embodiments of molecules having features of the invention, n is an integer between 1 and 3. In some embodiments of molecules having features of the invention, n is an integer between 1 and 2. In some embodiments of molecules having features of the invention, n is 1. In some embodiments of molecules having features of the invention, n is an integer of 1, 2, 3, 4, 5, 6, 7, 8, 9, 10, 11, 12, 13, 14, 15, 16, 17, 18, 19, or 20. In some embodiments of molecules having features of the invention, i is an integer between 1 and 20. In some embodiments of molecules having features of the invention, i is an integer between 1 and 10. In some embodiments of molecules having features of the invention, i is an integer between 1 and 5. In some embodiments of molecules having features of the invention, i is an integer between 1 and 4. In some embodiments of molecules having features of the invention, i is an integer between 1 and 3. In some embodiments of molecules having features of the invention, i is an integer between 1 and 2. In some embodiments of molecules having features of the invention, i is 1. In some embodiments of molecules having features of the invention, i is an integer of 1, 2, 3, 4, 5, 6, 7, 8, 9, 10, 11, 12, 13, 14, 15, 16, 17, 18, 19, or 20. In some embodiments, the T-Y pair is attached to either end of B.

In some embodiments, A is an antibody. In some embodiments, A is an anti-ErbB antibody. In some embodiments, A is an anti-HER2 antibody. In some embodiments, the antibody is an anti-EGFR antibody. In some embodiments, A is an antibody selected from the group consisting of cetuximab, trastuzumab, and pertruzumab. In some embodiments, A is cetuximab. In some embodiments, A is trastuzumab. In some embodiments, A is pertruzumab.

In some embodiments, the present invention provides an array comprising: a plurality of molecules of the structure according to one or more of the following:

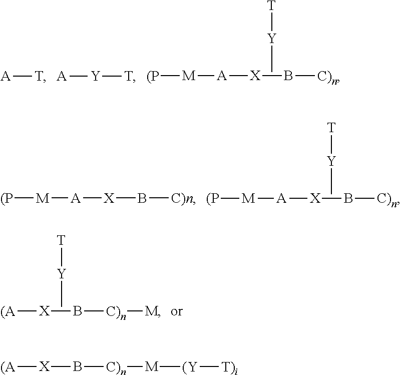

wherein cleavage of said molecule is indicative of the presence of protease activity and wherein P is an optional pre-targeting moiety; M is an optional macromolecular carrier; A is peptide portion with a sequence comprising about 2 to about 20 acidic amino acids, which when linked with portion B is effective to inhibit or prevent cellular uptake of portion B; B is a peptide portion of about 5 to about 20 basic amino acid residues, which is suitable for cellular uptake; X is a first cleavable linker of about 2 to about 100 atoms, or about 3 to about 30 atoms, joining A with B; Y is a second cleavable linker of about 2 to about 100 atoms, or about 3 to about 30 atoms, joining A-X-B with T; T is a compound for delivery to a target, including for example a therapeutic compound (also referred to herein as a “warhead”); and n is an integer between 1 and 20; and C is a detectable moiety. In some embodiments of the array, said portion A has between about 5 to about 9 acidic amino acid residues, and said portion B has between about 9 to about 16 basic amino acid residues. In some embodiments, the array comprises a plurality of molecules of one or more of said structures and wherein the cleavable linker X comprises a plurality of cleavable linkers X. In some embodiments, the array comprises a plurality of molecules of one or more of said structures and wherein the cleavable linker Y comprises a plurality of cleavable linkers Y. In some embodiments of the array, the plurality of cleavable linkers X and/or Y linking a portion A to a portion B are cleavable by a single protease. In some embodiments of the array, the plurality of cleavable linkers X and/or Y linking a portion A to a portion B are cleavable by more than one protease. In some embodiments, an array of the invention would contain a plurality of one species (one type) of MTS molecules. In some embodiments, an array of the invention would contain a plurality of one species (one type) of MTS molecules and multiple samples could be screened for one protease activity type. In some embodiments, an array of the invention would contain a plurality of a plurality of species (multiple types) of MTS molecules. In some embodiments, an array of the invention would contain a plurality of a plurality of species (multiple types) of MTS molecules and one or more samples could be screened for one or more protease activity types. An array can include but is not limited to any substrate to which the MTS molecules can be bound, and can include for examples solid substrates, micro-arrays and microchips. Methods for making arrays are well known and can even be supplied by commercial suppliers. Arrays can be manually processed and/or automated or a combination thereof. Such arrays can be employed in low-throughput as well as high-throughput applications and can analyze one or more samples, one or more proteases or any combination thereof. In some embodiments of molecules having features of the invention, n is an integer between 1 and 20. In some embodiments of molecules having features of the invention, n is an integer between 1 and 15. In some embodiments of molecules having features of the invention, n is an integer between 1 and 10. In some embodiments of molecules having features of the invention, n is an integer between 1 and 5. In some embodiments of molecules having features of the invention, n is an integer between 1 and 4. In some embodiments of molecules having features of the invention, n is an integer between 1 and 3. In some embodiments of molecules having features of the invention, n is an integer between 1 and 2. In some embodiments of molecules having features of the invention, n is 1. In some embodiments of molecules having features of the invention, n is an integer of 1, 2, 3, 4, 5, 6, 7, 8, 9, 10, 11, 12, 13, 14, 15, 16, 17, 18, 19, or 20. In some embodiments of molecules having features of the invention, i is an integer between 1 and 20. In some embodiments of molecules having features of the invention, i is an integer between 1 and 10. In some embodiments of molecules having features of the invention, i is an integer between 1 and 5. In some embodiments of molecules having features of the invention, i is an integer between 1 and 4. In some embodiments of molecules having features of the invention, i is an integer between 1 and 3. In some embodiments of molecules having features of the invention, i is an integer between 1 and 2. In some embodiments of molecules having features of the invention, i is 1. In some embodiments of molecules having features of the invention, i is an integer of 1, 2, 3, 4, 5, 6, 7, 8, 9, 10, 11, 12, 13, 14, 15, 16, 17, 18, 19, or 20. In some embodiments, the T-Y pair is attached to either end of B.

In some embodiments of the above described methods, ratiometric analysis can be employed to determine the level of enzyme activity and/or to assess the percentage of enzymatically positive tumors in a population. Such ratiometric analyses can be based on the ratio of cleaved to non-cleaved MTS molecules. In some embodiments, ratiometric analysis can be employed to correlate ex vivo cleavage with in vivo cleavage activities.

In some embodiments, A is an antibody. In some embodiments, A is an anti-ErbB antibody. In some embodiments, A is an anti-HER2 antibody. In some embodiments, the antibody is an anti-EGFR antibody. In some embodiments, A is an antibody selected from the group consisting of cetuximab, trastuzumab, and pertruzumab. In some embodiments, A is cetuximab. In some embodiments, A is trastuzumab. In some embodiments, A is pertruzumab.

In some embodiments, the protease information can be correlated with histology, grade, type, characterization, etc. in order to better characterize neoplasias and to provide personalized prognosis and treatment regimens. Such information can be provided to those of skill in the medical arts and be employed to develop personalized medical treatment regimens for individuals.

### Radiosensitization

The present invention also provides for methods of employing the MTS molecules of the present invention in radiosensitization. As used herein, a radiosensitizer is a drug that induces tumor cells more sensitive to radiation therapy. Such a radiosensitzer can allow for increase tumor cell death. Such a radiosensitzer can allow for reduced tumor cell growth. Such a radiosensitzer can allow for a reduction in the amount of radiation therapy need for a therapeutic effect.

In some embodiments, T is a radiosensitizing entity. Radiosensitizing drugs can include any substance that when administered to a tumor or cancer cell leads to increased sensitivity or susceptibility of the tumor or cancer cell to treatment with radiation therapy; also referred to as radiosensitizing agents. In some embodiments, T is an entity as described herein above.

Exemplary radiosensitizing agents can include but are not limited to any of the chemotherapeutic agents described herein above. In some embodiments, T is DM1 (mertansine) or a derivative thereof. In some embodiments, T is a radiosensitizing agent. In some embodiments, the radiosensitizing agent is not an auristatin, including MMAE. In some embodiments, T is a maytansinoid. In some embodiments, T is mertansine.

In some embodiments, the MTS molecules contain 1, 2, 3, 4, 5, 6, or more different T entities capable of radiosensitization. In some embodiments, a portion A (such as an antibody) delivers the compound T (e.g., radiosensitizing agent T) to tumor. In some embodiments, a portion A (such as an antibody) delivers the compound T (e.g., radiosensitizing agent T) to tumor and the tumor is sensitized to the radiosensitizing agent T.

In some embodiments, the MTS molecules containing 1, 2, 3, 4, 5, 6, or more different T entities capable of radiosensitization are administered to an individual with a cancer or tumor. In some embodiments, the MTS molecules containing 1, 2, 3, 4, 5, 6, or more different T entities capable of radiosensitization are administered to an individual with a cancer or tumor prior to administration of radiation therapy. In some embodiments, the MTS molecules containing 1, 2, 3, 4, 5, 6, or more different T entities capable of radiosensitization are administered to an individual with a cancer or tumor concurrently with administration of radiation therapy. In some embodiments, the radiation therapy can be administered before the MTS molecule. In some embodiments, the radiation therapy can be administered after the MTS molecule. Radiation therapy can be administered according to established standards of care, including dosages of 20-40 gray (Gy), 45-60 Gy, 60-80 Gy, as needed and as determined by a skilled physician in the art. In some embodiments, the radiation dosage is administered as a single dosage or as multiple dosages. In some embodiments, the radiation therapy can be administered 1.8 to 2 Gy per day or 2.67 to 2.75 Gy per day. In some embodiments, the radiation therapy can be administered 1, 2, 3, 4, or 5 days per week, including 1-5, 2-5, 3-5, or 4-5 days per week. In some embodiments, the radiation therapy can be administered 1 to 2 days per week. In some embodiments, a portion A (such as an antibody) delivers the compound T (e.g., radiosensitizing agent T) to tumor in combination with radiation therapy (also referred to as ionizing radiation or ionizing radiation therapy).

### Imaging

A variety of methods of imaging fluorescent compounds are known in the art and those of skill in the art will understand application of such methods in the context of imaging the compounds of the present invention according to the methods described herein.

Frozen block face imaging provides fluorescence maps of where the Cy5-labeled mAb or ACPP is ending up in a tumor and its surrounding tissue with minimal perturbation or manipulation to signal. In some embodiments, the tissue is fresh-frozen. In some embodiments, the freezing is sufficiently fast or rapid such as to minimize cellular damage. In some embodiments, there are no perfusion or fixation artifacts. In some embodiments, the absence of perfusion or fixation artifacts allows the signal in the blood and vessels to be preserved. In some embodiments, a block face is cut, up to a centimeter across. Such a cross section can then be imaged. In some embodiments, such imaging is on a Nikon confocal microscope. In some embodiments, the imaging occurs while the sample is still frozen. In some embodiments, the sample is insulated to keep the sample frozen.

Multiple tumor models are available and known to those in the art with GFP and others that can be labeled with an RFP. In some embodiments, tumors models labeled with GFP or RFP allow for visualization of the same sample on both a macro and a micro scale. In some embodiments, this will allow for a determination of whether the mAb or ACPP enters into the fluorescent tumor cells or is primarily confined to the tumor stroma. See, FIG. 46A-D for an exemplary comparison of Cy5 fluorescence showing different localizations of a mAb, cetuximab as compared to an ACPP (PLGC(Me)AG Cy5/Cy7-labeled RACPP).

In some embodiments, frozen block face imaging can be employed. In some embodiments, such imaging methods allow for a determination of the relative amounts of mAb or ACPP (MTS) loaded into tumor cells versus host macrophages and examination of the vascular endothelium. In some embodiments, such methods allow for identification of specific cell types in the stroma, including for example but not limited to macrophages, neutrophils and mast cells using a variety of labeled antibodies.

In some embodiments, mAb or ACPP uptake into tumor versus host cells can be compared using fixed tissue and a variety of antibodies against cell markers of interest (for example, in order to determine cell identity). In some embodiments, the tissue can be stained with Hoechst or other nuclear markers after chemoradiation treatment to assess relative survival rates of malignant cells as compared components of stroma and vasculature.

### Imaging Methods for Warhead Distribution

In some embodiments Mass Spectrometry Imaging (MSI) can be employed to quantitatively measure warhead loading into biological tissue (see, Laskin J, et al., Anal Chem. 2016; 88(1):52-73; and Hsu C C, Dorrestein P C. Clin Chem. 2016; 62(1):111-23).

In some embodiments, mass spectrometry can be employed to record the spatial distribution of drug compound (i.e., Cargo C, compound T, radiosensitizing agent T, and/or “warhead”) allows the loading to be observed in the absence of fluorescent tags. In such embodiments, the signal is an orthogonal signal to epifluorescence microscopy, even though the sample is prepared for the instrument in a similar way. In some embodiments, a frozen slice of tissue is thaw mounted on a glass slide. In some embodiments, the slice of tissue is about 5 μm, about 7.5 μm, about 10 μm, about 12.4 μm, or about ˜15 μm thick. In some embodiments, the tissue is then placed in the mass spectrometer (MS) interface. In some embodiments, the tissue is then imaged by measuring discrete mass spectra over each pixel, in accordance with the spatial resolution. The type of MS interface is the main factor determining the spatial resolution.

In some embodiments, matrix assisted laser desorption time-of-flight mass spectrometry (MALDI-TOF) (Chaurand P, et al., Curr Opin Biotechnol. 2006; 17(4):431-6) can be employed for imaging purposes according to the present invention. In some embodiments, Desorption Electrospray Ionization (DESI), developed by Cooks (Cooks R G, et al., Science. 2006; 311(5767):1566-70.) can be employed for imaging purposes according to the present invention. In some embodiments, DESI and related techniques, including nanoflow DESI (nanoDESI) are classed as ambient MSI (Laskin J, Lanekoff, I. Anal Chem. 2016; 88(1):52-73) because the sample is ionized at ambient pressure from a small liquid droplet formed on the tissue when mounted on a standard glass slide. The spatial resolution in nanoDESI is determined by the diameter of the droplet formed between two fused silica capillaries, currently ˜150 μm. In some embodiments of these methods, the chemical components in the tissue are extracted into the droplet and introduced into the MS by nanoflow electrospray ionization.

In some embodiments, nanoDESI (Lanekoff I, et al., Anal Chem. 2012; 84(19):8351-6) can be employed to address where a warhead, a molecule T such as for example, MMAE, travels to the heterogeneous microenvironment of a tumor. In some embodiments, nanoDesi can be employed to determine the distribution of a molecule T. In some embodiments, a cross section of a sample, after injection of an ACPP (MTS) is examined. In some embodiments, the section is about 5 μm, about 10 μm, about 15 μm, about 20 μm, about 30 μm, about 40 μm, about 45 μm, about 50 μm, about 55 μm, about 60 μm, or about 65 μm. In some embodiments, the cross section is about 40 μm, about 45 μm, about 50 μm, about 55 μm, or about 60 μm. In some embodiments, the cross section is about 50 μm. In some embodiments, an apparatus as shown in FIG. 47 is employed. In some embodiments, the parent molecular ion forms from the intact ACPP (MTS) and is measured. In some embodiments, the characteristic fragment ion is formed from a molecular cleavage that occurs in the ionization process and is measured. In some embodiments, both ions can be measured and used for quantitation. In some embodiments, the tumor can be categorized based on the cleavage of the ACPP (MTS). In some embodiments, the tumor microenvironment can be categorized based on the cleavage of the ACPP (MTS). For example, the proteases expressed by the tumor can be determined by employing an ACPP (MTS) which contains protease cleavable linker which is only cleavable by a particular protease, such as MMP-9 or MMP-2, etc.

In some embodiments, lipids from cell membranes and extracellular spaces occur as negative ions in nanoDESI. In some embodiments, analytes that contain secondary amines or pyridyl groups in their structures are detected with good sensitivity as positive ions. In some embodiments, conditions are determined and employed to prevent suppression of the analyte ions by electrostatic attraction from ionized lipids. In such embodiments, the solvent for extracting the sample is first continuously flowed over the point of contact with the sample, so its organic content can be systematically varied while the mass spectral results are observed. In such embodiments, modifying reagents can be added secondarily to the solvent stream if desired, for example, to adjust the pH or other solubility parameters.

In some embodiments, NanoDESI is employed for quantitation, because the standard can be added in the flowing solvent (see, for example, Laskin J, Lanekoff I., Anal Chem. 2016; 88(1):52-73). In some embodiments, a second standard can also be added directly to the sample slice, to control for extraction efficiency. In some embodiments, two standards are thus used to calibrate for extraction and ionization independently. In such embodiments employed two standards, it is not as essential to match the properties of the analyte exactly. In some embodiments, this allows for avoidance of the synthesis of drug targets substituted with stable isotope atoms. In some embodiments, these calibration methods are semi-quantitative. In some embodiments, the semi-quantitativeness of the calibration methods depends on factors such as the consistency of solvent flow. In some embodiments, the average drug concentration in the tissue will be measured from bulk tissue homogenates. In some embodiments, the overall result will be compared with the distribution found in tissue in order to quantitatively determine the amount of compound T in a tissue. In some embodiments, the amount of compound T is a tissue can be determined.

In some embodiments, the present invention provides a method for imaging a tumor sample comprising:

i) administering to a subject in need thereof a molecule comprising the formula:

A-T

wherein the molecule comprises:

an antibody A;

a radiosensitizing agent T, wherein said radiosensitizing agent is not an auristatin, including MMAE,

ii) obtaining a sample from the subject; and
iii) determining the location of compound T, using an imaging method.

In some embodiments of the method, the molecule is cleaved in vivo in the presence of a tumor in the subject.

In some embodiments of the method, the imaging method is NanoDESI.

In some embodiments of the method, determining the location of compound T allows for a determination of the tumor type and/or a categorization of the tumor microenvironment.

In some embodiments, the present invention provides a method for imaging a tumor sample comprising:

i) administering to a subject in need thereof a molecule comprising the formula:

A-Y-T

wherein the molecule comprises:

an antibody A;

a cleavable linker Y;

a radiosensitizing agent T, wherein said radiosensitizing agent is not an auristatin, including MMAE,

wherein said subject is radiosensitized.

In some embodiments of the method, the molecule is cleaved in vivo in the presence of a tumor in the subject.

In some embodiments of the method, the imaging method is NanoDESI.

In some embodiments of the method, determining the location of compound T allows for a determination of the tumor type and/or a categorization of the tumor microenvironment.

In some embodiments, the present invention provides a method for imaging a tumor sample comprising:

i) administering to a subject in need thereof a molecule comprising the formula:

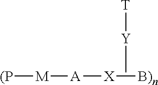

wherein the molecule comprises:

an optional pre-targeting moiety P;

an optional macromolecular carrier M;

a peptide A with a sequence comprising 5 to 9 consecutive acidic amino acids, wherein the amino acids are selected from: aspartates and glutamates;

a first cleavable linker X;

a second cleavable linker Y;

a radiosensitizing agent T;

a peptide B with a sequence comprising 5 to 20 consecutive basic amino acids;

the T-Y pair is attached to either end of B; and

n is an integer between 1 and 20;

ii) obtaining a sample from the subject; and
iii) determining the location of compound T, using an imaging method.

In some embodiments of the method, the molecule is cleaved in vivo in the presence of a tumor in the subject.

In some embodiments of the method, the imaging method is NanoDESI.

In some embodiments of the method, determining the location of compound T allows for a determination of the tumor type and/or a categorization of the tumor microenvironment.

In some embodiments, the present invention provides a method for imaging a tumor sample comprising:

i) administering to a subject in need thereof a molecule comprising the formula:

(A-X-B)ₙ-M -(Y-T)ᵢ

wherein the molecule comprises:

a macromolecular carrier M bound to A or B;

a peptide A with a sequence comprising 5 to 9 consecutive acidic amino acids, wherein the amino acids are selected from: aspartates and glutamates;

a first cleavable linker X;

a second cleavable linker Y;

a compound T;

a peptide B with a sequence comprising 5 to 20 consecutive basic amino acids; and

n and i are independently selected integers between 1 and 20;

ii) obtaining a sample from the subject; and
iii) determining the location of compound T, using an imaging method.

In some embodiments of the method, the molecule is cleaved in vivo in the presence of a tumor in the subject.

In some embodiments of the method, the imaging method is NanoDESI.

In some embodiments of the method, determining the location of compound T allows for a determination of the tumor type and/or a categorization of the tumor microenvironment.

In some embodiments, the present invention provides a method for imaging a tumor sample comprising:

i) subjecting a sample from a subject to a molecule comprising the formula:

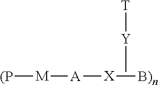

wherein the molecule comprises:

an optional pre-targeting moiety P;

an optional macromolecular carrier M;

a peptide A with a sequence comprising 5 to 9 consecutive acidic amino acids, wherein the amino acids are selected from: aspartates and glutamates;

a first cleavable linker X;

a second cleavable linker Y;

a radiosensitizing agent T;

a peptide B with a sequence comprising 5 to 20 consecutive basic amino acids;

the T-Y pair is attached to either end of B; and
n is an integer between 1 and 20; and
   ii) determining the location of compound T, using an imaging method.

In some embodiments of the method, the molecule is cleaved in vitro in the presence of a tumor sample.

In some embodiments, the imaging method is a fluorescent based imaging method. In some embodiments of the method, the imaging method is NanoDESI, MRI, or PET (positron-emission tomography).

In some embodiments of the method, determining the location of compound T allows for a determination of the tumor type and/or a categorization of the tumor microenvironment.

In some embodiments, the present invention provides a method for imaging a tumor sample comprising:

i) subjecting a sample from a subject in need thereof a molecule comprising the formula:

(A-X-B)ₙ-M-(Y-T)ᵢ

wherein the molecule comprises:

a macromolecular carrier M bound to A or B;

a peptide A with a sequence comprising 5 to 9 consecutive acidic amino acids, wherein the amino acids are selected from: aspartates and glutamates;

a first cleavable linker X;

a second cleavable linker Y;

a compound T;

a peptide B with a sequence comprising 5 to 20 consecutive basic amino acids; and
n and i are independently selected integers between 1 and 20; and
   ii) determining the location of compound T, using an imaging method.

In some embodiments of the method, the molecule is cleaved in vitro in the presence of a tumor sample.

In some embodiments, the imaging method is a fluorescent based imaging method. In some embodiments of the method, the imaging method is NanoDESI, MM (magnetic resonance imaging), or PET (positron-emission tomography). Such imaging methods can be used with the MTS molecules of the present invention.

In some embodiments of the method, determining the location of compound T allows for a determination of the tumor type and/or a categorization of the tumor microenvironment.

### Pharmaceutical Compositions

The MTS molecules of the present invention can be administered with a suitable pharmaceutical excipient and/or buffer as necessary. One of skill will understand that the composition will vary depending on mode of administration and dosage unit.

The compositions typically include a conventional pharmaceutical carrier, buffer, or excipient and may additionally include other medicinal agents, carriers, adjuvants, diluents, tissue permeation enhancers, solubilizers, and the like. Preferably, the composition will contain about 0.01% to about 90%, about 0.1% to about 75%, about 0.1% to 50%, or about 0.1% to 10% by weight of a conjugate of the present invention or a combination thereof, with the remainder consisting of suitable pharmaceutical carrier and/or excipients. Appropriate excipients can be tailored to the particular composition and route of administration by methods well known in the art. See, e.g., REMINGTON'S PHARMACEUTICAL SCIENCES, 18TH ED., Mack Publishing Co., Easton, Pa. (1990).

Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, and polyacrylic acids such as Carbopols, e.g., Carbopol 941, Carbopol 980, Carbopol 981, etc. The compositions can additionally include lubricating agents such as talc, magnesium stearate, and mineral oil; wetting agents; emulsifying agents; suspending agents; preserving agents such as methyl-, ethyl-, and propyl-hydroxy-benzoates (i.e., the parabens); pH adjusting agents such as inorganic and organic acids and bases; sweetening agents; coloring agents; and flavoring agents. The compositions may also comprise biodegradable polymer beads, dextran, and cyclodextrin inclusion complexes.

For oral administration, the compositions can be in the form of tablets, lozenges, capsules, emulsions, suspensions, solutions, syrups, sprays, powders, and sustained-release formulations. Suitable excipients for oral administration include pharmaceutical grades of mannitol, lactose, starch, magnesium stearate, sodium saccharine, talcum, cellulose, glucose, gelatin, sucrose, magnesium carbonate, and the like.

In some embodiments, the pharmaceutical compositions take the form of a pill, tablet, or capsule, and thus, the composition can contain, along with the conjugate or combination of conjugates, any of the following: a diluent such as lactose, sucrose, dicalcium phosphate, and the like; a disintegrant such as starch or derivatives thereof; a lubricant such as magnesium stearate and the like; and a binder such a starch, gum acacia, polyvinylpyrrolidone, gelatin, cellulose and derivatives thereof. The conjugates can also be formulated into a suppository disposed, for example, in a polyethylene glycol (PEG) carrier.

Liquid compositions can be prepared by dissolving or dispersing a conjugate or a combination of conjugates and optionally one or more pharmaceutically acceptable adjuvants in a carrier such as, for example, aqueous saline (e.g., 0.9% w/v sodium chloride), aqueous dextrose, glycerol, ethanol, and the like, to form a solution or suspension, e.g., for oral, topical, or intravenous administration. The conjugates of the present invention can also be formulated into a retention enema.

For topical administration, the compositions of the present invention can be in the form of emulsions, lotions, gels, creams, jellies, solutions, suspensions, ointments, and transdermal patches. For delivery by inhalation, the composition can be delivered as a dry powder or in liquid form via a nebulizer. For parenteral administration, the compositions can be in the form of sterile injectable solutions and sterile packaged powders. Preferably, injectable solutions are formulated at a pH of about 4.5 to about 7.5.

The compositions of the present invention can also be provided in a lyophilized form. Such compositions may include a buffer, e.g., bicarbonate, for reconstitution prior to administration or the buffer may be included in the lyophilized composition for reconstitution with, e.g., water. The lyophilized composition may further comprise a suitable vasoconstrictor, e.g., epinephrine. The lyophilized composition can be provided in a syringe, optionally packaged in combination with the buffer for reconstitution, such that the reconstituted composition can be immediately administered to a patient.

One of ordinary skill in the art understands that the dose administered will vary depending on a number of factors, including, but not limited to, the particular peptide composition to be administered, the mode of administration, the type of application (e.g., prophylactic, therapeutic, etc.), the age of the patient, and the physical condition of the patient. Preferably, the smallest dose and concentration required to produce the desired result should be used. Dosage can be appropriately adjusted for children, the elderly, debilitated patients, and patients with cardiac and/or liver disease. Further guidance can be obtained from studies known in the art using experimental animal models for evaluating dosage. The MTS molecules of the present invention can be formulated without undue experimentation for administration to a mammal, including humans, as appropriate for the particular application. Additionally, proper dosages of the compositions can be determined without undue experimentation using standard dose-response protocols.

In some embodiments, the peptide comprising compositions of the present invention are administered to a subject at a particular dose of the peptide or are formulated for unit dosage administration of the peptide to a subject. In some embodiments, the dose administered to a subject is from about 0.001 to about 1000 mg per day. In some embodiments, the dose administered to a subject is from about 0.1 to about 500 mg per day. In some embodiments, the dose administered to a subject is from about 0.5 to about 100 mg per day. In some embodiments, the compositions of the present invention are formulated for unit dosage administration, wherein the unit dosage is from about 0.001 to about 1000 mg per day. In some embodiments, the compositions of the present invention are formulated for unit dosage administration, wherein the unit dosage is from about 0.1 to about 500 mg per day. In some embodiments, the compositions of the present invention are formulated for unit dosage administration, wherein the unit dosage is from about 0.5 to about 100 mg per day.

### Methods of Administration

Administration of the peptides of the present invention with a suitable pharmaceutical excipient as necessary can be carried out via any of the accepted modes of administration. Thus, administration can be, for example, intravenous, topical, subcutaneous, transcutaneous, transdermal, intramuscular, oral, intra joint, parenteral, intra-arteriole, intradermal, intraventricular, intracranial, intraperitoneal, intralesional, intranasal, rectal, vaginal, or by inhalation. Administration can be targeted directly to pancreatic tissue, e.g., via injection.

The MTS molecule compositions of the invention may be administered repeatedly, e.g., at least 2, 3, 4, 5, 6, 7, 8, or more times, or the composition may be administered by continuous infusion. Suitable sites of administration include, but are not limited to, dermal, mucosal, bronchial, gastrointestinal, anal, vaginal, eye, and ear. The formulations may take the form of solid, semi-solid, lyophilized powder, or liquid dosage forms, such as, for example, tablets, pills, lozenges, capsules, powders, solutions, suspensions, emulsions, suppositories, retention enemas, creams, ointments, lotions, gels, aerosols, or the like, preferably in unit dosage forms suitable for simple administration of precise dosages.

The term “unit dosage form” refers to physically discrete units suitable as unitary dosages for human subjects, each unit containing a predetermined quantity of active material calculated to produce the desired onset, tolerability, and/or therapeutic effects, in association with a suitable pharmaceutical excipient (e.g., an ampoule). In addition, more concentrated compositions may be prepared, from which the more dilute unit dosage compositions may then be produced. The more concentrated compositions thus will contain substantially more than, e.g., at least 1, 2, 3, 4, 5, 6, 7, 8, 9, 10, or more times the amount of a conjugate or a combination of conjugates.

Methods for preparing such dosage forms are known to those skilled in the art (see, for example, REMINGTON'S PHARMACEUTICAL SCIENCES, 18TH ED., Mack Publishing Co., Easton, Pa. (1990). The composition to be administered contains a quantity of the peptides of the invention in a pharmaceutically effective amount for improving beta islet cell survival. In addition, pharmaceutically acceptable salts of the peptides of the present invention (e.g., acid addition salts) may be prepared and included in the compositions using standard procedures known to those skilled in the art of synthetic organic chemistry and described, e.g., by March, Advanced Organic Chemistry: Reactions, Mechanisms and Structure, 4th Ed., New York, Wiley-Interscience (1992).

### EXAMPLES

The methods system herein described are further illustrated in the following examples, which are provided by way of illustration and are not intended to be limiting.

### Example 1: Anti-Tubulin Drugs Conjugated to Anti-ErbB Antibodies Selectively Radiosensitize

Tumour resistance to radiotherapy remains a barrier to improving cancer patient outcomes. To overcome radioresistance, certain drugs have been found to sensitize cells to ionizing radiation (IR). In theory, more potent radiosensitizing drugs should increase tumour kill and improve patient outcomes. In practice, clinical utility of potent radiosensitizing drugs is curtailed by off-target side effects. Here we report potent anti-tubulin drugs conjugated to anti-ErbB antibodies selectively radiosensitize to tumours based on surface receptor expression. While two classes of potent anti-tubulins, auristatins and maytansinoids, indiscriminately radiosensitize tumour cells, conjugating these potent anti-tubulins to anti-ErbB antibodies restrict their radiosensitizing capacity. Of translational significance, we report that a clinically used maytansinoid ADC, ado-trastuzumab emtansine (T-DM1), with IR prolongs tumour control in target expressing HER2+ tumours but not target negative tumours. In contrast to ErbB signal inhibition, our findings establish an alternative therapeutic paradigm for ErbB-based radiosensitization using antibodies to restrict radiosensitizer delivery.

Concurrent chemotherapy and radiotherapy form the basis of curative organ sparing therapy for patients with locally advanced cancers′. Chemotherapy not only has intrinsic anti-tumour activity but can sometimes sensitize tumours to radiation kill. The 1990s saw multiple randomized trials unequivocally demonstrate combining cytotoxic chemotherapy (that is, cisplatin, 5-fluorouracil and taxanes) with radiotherapy to improve tumour control and patient survival′. However, the morbidity of such intensive regimens precludes development of more potent radiosensitizing chemotherapies′. Shockingly two decades later, non-targeted cytotoxic chemotherapies continue to remain the most effective approach for patients treated with concurrent chemo-radiotherapy.

To be clinically useful, radiosensitizing chemotherapies must improve the therapeutic index, that is, the level of tumour cell sensitization must be greater than surrounding normal tissue¹⁰⁻¹². In theory, molecularly targeted radiosensitizers blocking tumour-specific pathways should increase the therapeutic index of IR by improving tumour control and decreasing side effects. Identification of ErbB (EGFR, HER2) playing a role in tumour radioresistance has led to attempts to sensitize tumours by inhibiting receptor signalling¹³⁻²⁰. However, the efficacy of ErbB signal inhibition is limited because tumours have parallel signalling pathways circumventing the blockade²¹⁻²⁵. Antibody drug conjugates (ADC) are emerging as a tumour targeted delivery strategy to restrict localization of drugs to tumours while sparing normal tissue²⁶⁻²⁸. ADC consist of a drug (warhead) covalently attached to an antibody recognizing a specific cell surface receptor. ADC binds to cells expressing the receptor, is then internalized by receptor-mediated endocytosis, and finally the drug is released from the antibody by the action of endolysosomal proteases. Maytansinoids and auristatins are potent anti-tubulin drugs that have been conjugated to antibodies with demonstrated clinical efficacy²⁹⁻³². Importantly, we have recently discovered that monomethyl auristatin E (MMAE) is a radiosensitizer, effective at the single nM level³³.

We hypothesized that therapeutic antibodies to ErbB receptors could direct delivery of highly potent anti-tubulin drugs in a receptor-restricted manner to selectively radiosensitize tumours. To test this hypothesis in tumour model systems, we initially synthesized two ADC in which the anti-tubulin drug monomethyl auristatin E was conjugated to cetuximab or trastuzumab (C-MMAE and T-MMAE, respectively). C-MMAE and T-MMAE bound and restricted MMAE activity and toxicity to EGFR and HER2 expressing tumour cells, respectively. Importantly while free MMAE radiosensitized indiscriminately, antibody conjugation resulted in targeted MMAE radiosensitiza-tion to EGFR or HER2 expressing tumours. To delineate the translational potential of these findings, we extended our studies to the clinically approved anti-tubulin ADC, ado-trastuzumab emtansine (T-DM1). We found that T-DM1 radiosensitized HER2 expressing tumours specifically resulting in significantly increased tumour xenograft control. On the basis of these findings, we propose antibody drug conjugate based chemo-radiotherapy paradigms designed to focus on antibody directed delivery of highly potent radiosensitizing chemotherapies as an alternative to receptor signal inhibition.

### Results

Efficacy of Anti-ErbB Antibodies Conjugated to Cy5 and MMAE.

To test if ADC can restrict MMAE radiosensitization to tumours, we conjugated MMAE to cetuximab (C-MMAE) and trastuzumab (T-MMAE) and labelled them with Cy5 for tracking (Supplementary FIGS. 1a and 2). Cetuximab and trastuzumab were labelled at endogenous cysteines by selective reduction of the four disulfides in the hinge region and conjugation confirmed by ES-HPLC, with drug loading measured as ˜3.7 and ˜3.2 MMAE per molecule of cetuximab and trastuzumab, respectively and with ˜1 Cy5 (refs 34,35). We used thiol-reactive maleimide derivatives of MMAE containing cathepsin-B cleavable valine-citrulline linkers that are present in the clinically approved ADC, brentuximab vedotin. We first evaluated the functionality of C-MMAE and T-MMAE. EGFR expressing CAL-27 head and neck cancer (HNC) cells were treated with C-MMAE and imaged by direct fluorescence (FIG. 1a, Supplementary FIG. 3a). By 30 min, Cy5 fluorescence localized to the cell surface and also was internalized. We then tested the specificity of C-MMAE and T-MMAE in a panel of cancer cell lines from different histologies treated with chemo-radiotherapy, HNC, non-small cell lung cancer (NSCLC) and esophageal (FIG. 1b, Supplementary FIG. 3a, Supplementary Table 1). C-MMAE bound to EGFR expressing CAL-27, A549 and CALU3 cells with decreasing affinity. T-MMAE demonstrated high affinity to the HER2 expressing cell lines CALU3, OE19 and BT474. Confocal microscopy results were validated by measuring cell surface binding of C-MMAE and T-MMAE (FIG. 1c, Supplementary FIG. 3b). CAL-27 cells bound to C-MMAE in a dose-dependent manner but not T-MMAE. In contrast, HER2 expressing cell lines, OE19, CALU3, and BT474 demonstrated dose-dependent binding of T-MMAE. We next assessed if C-MMAE and T-MMAE retained MMAE functional activity of arresting cells in G₂/M. Morphologically, CAL-27 cells treated with either free MMAE or C-MMAE appeared identical with cells rounding up, indicative of G₂/M (FIG. 1d). OE19 and CAL-27 cells treated with free MMAE resulted in cells accumulating in G₂/M, the most radiosensitive phase of the cell cycle (FIG. 1e, Supplementary FIG. 4). In OE19 cells, T-MMAE blocked cells in G₂/M in contrast to C-MMAE or antibodies alone which did not influence the cell cycle profile. In CAL-27 cells, C-MMAE resulted in G₂/M arrest. Interestingly, our Cy5-labelled C-MMAE allowed discrimination of EGFR cell surface receptor availability that directly correlated with the potency of C-MMAE to free MMAE in EGFR expressing cell lines (Supplementary FIG. 5).

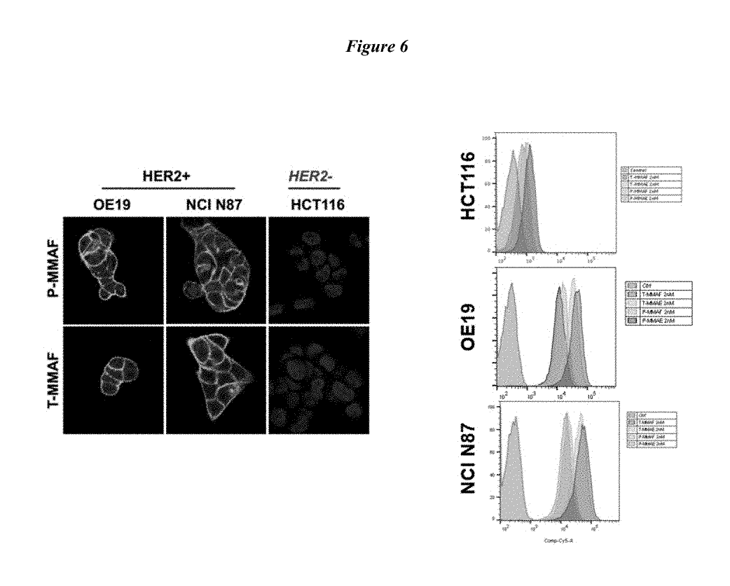

Antibody conjugation restricts MMAE radiosensitization. Key advantages driving the clinical development of MMAE are its potency and its ability to be tumour targeted through antibody conjugation^{27,28}. In cell lines we tested, MMAE was more potent compared with standard cytotoxic chemotherapies that are used concurrently with radiotherapy in patients, that is, cisplatin and paditaxel (FIG. 2a)³³. Importantly, C-MMAE or T-MMAE restricted MMAE cytotoxicity to EGFR or HER2 expressing cells, respectively, and were much more potent than antibodies alone (FIG. 2b). Since concurrent chemo-radiotherapy is standard of care in HNC, NSCLC and esophageal cancers, we tested whether ADC restricted MMAE radiosensitization in cell lines of these histologies¹⁻³. Following IR, cells die predominantly as a result of mitotic catastrophe that can be measured by donogenic survival¹¹. CAL-27 cells were treated overnight with MMAE, cetuximab or C-MMAE and then irradiated. The surviving fraction following 2 Gy decreased in cells treated with free MMAE or C-MMAE compared with vehicle or cetuximab-treated cells (FIG. 2c). At these lower drug concentrations, cetuximab had no radiosensitizing effect. Mechanistically, IR induces cell death by causing DNA double strand breaks, which can be measured by neutral comet assay¹¹. Irradiation of vehicle-treated CAL-27 cells resulted in a 3.6-fold increase in comet tail length that was significantly increased by 0.5 nM C-MMAE but not by 5 nM cetuximab (FIG. 2d). Importantly, C-MMAE restricted MMAE radiosensitization. In low EGFR binding LN229 cells (FIG. 2e, Supplementary FIG. 6), irradiation resulted in a 2.5-fold increase in comet tail length that was not appreciably increased by 20 nM cetuximab or C-MMAE (FIG. 2d). Interestingly in irradiated CALU3 cells, 2 nM of free MMAE, C-MMAE or T-MMAE all resulted in increased comet tail length to a similar degree when compared with vehicle or 20 nM antibody-treated cells (FIG. 2f).

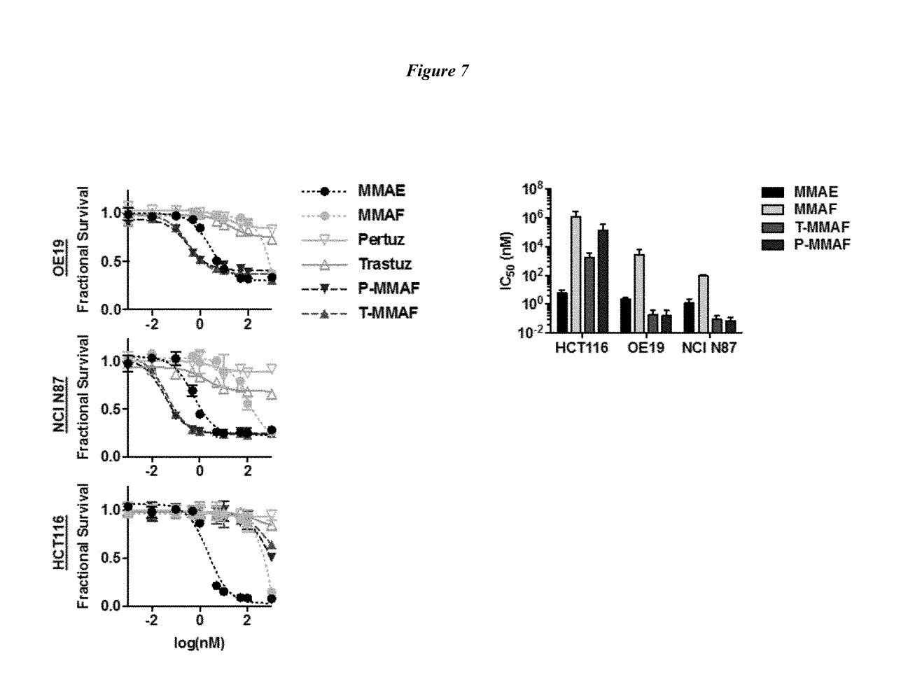

This result is concordant with cytotoxicity data, which demonstrated C-MMAE and T-MMAE were equally cytotoxic in CALU3 cells (Supplementary FIG. 7). In irradiated HER2 OE19 cells, 2 nM free MMAE or T-MMAE further increased comet tail length compared vehicle, 20 nM antibody or 2 nM C-MMAE treated cells (FIG. 2f).

Maytansinoids Radiosensitize and can be HER2 Targeted.

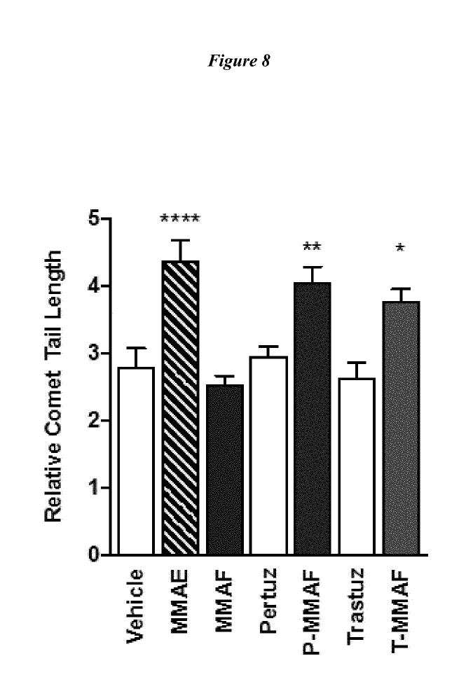

Similar to the auristatins (for example, MMAE), maytansinoids (for example, mertansine) are another potent class of anti-tubulins²⁹. Importantly, mertansine forms the warhead of the trastuzumab ADC T-DM1, and is attached to lysine residues of trastuzumab through a thiol ether linker (Supplementary FIG. 1b). T-DM1 (Kadcyla) has shown efficacy in HER2 expressing metastatic breast cancer patients^{29,30}. Given our findings that T-MMAE selectively radiosensitized HER2 expressing cancer cells, we decided to evaluate the more immediate clinical potential of our strategy by testing if T-DM1 radiosensitized tumours in a HER2+selective manner. First, we tested if the maytansinoid warhead of T-DM1 was a bona fide radiosensitizer. HCT116 or CAL-27 cells were treated overnight with mertansine and then irradiated with 2 Gy. In both cell lines, the surviving fraction following 2 Gy decreased further in cells treated with free mertansine compared with vehicle (FIG. 3a). Irrespective of HER2 expression, the IC₅₀ of mertansine was fairly consistent across cell lines 10 nM (FIG. 3b). In contrast, T-DM1 potency directly correlated with HER2 expression. In HER2 cell lines (OE19 and NCI N87 (gastric cancer)) the IC₅₀ of T-DM1 was <1 nM but was >100 nM in HER2− cells(HCT116 and CAL-27). From a translation point of view, T-DM1 was a more potent cytotoxic drug in HER2+OE19 cells compared with clinically used radiosensitizers that are either cytotoxic (paditaxel and cisplatin) or targeted to HER2 (trastuzumab and lapatinib) or EGFR (erlotinib) (FIG. 3c). However in HER2− CAL-27 cells, paclitaxel was more potent than T-DM1 (Supplementary FIG. 8). Functionally, mertansine increased the accumulation of both OE19 and HCT116 cells in the radiosensitive G₂/M phase of the cell cycle (FIG. 3d). While T-DM1 also blocked OE19 cells in G₂/M, the cell cycle profile of HCT16 cells treated with T-DM1 resembled vehide-treated cells. Next, we tested if T-DM1 would restrict mertansine mediated radiosensitization in a HER2-dependent manner. Unconjugated mertansine (20 nM) radiosensitized by increasing IR induced DNA double-strand breaks in both HER2+ and HER2− cell lines (FIG. 3e). Importantly, 20 nM T-DM1 resulted in restricted radiosensitization of HER2+cells. As with T-DM1's increased potency and ability to arrest cells in G₂/M confined to HER2+cells, trastuzumab conjugation reversed mertansine's ability to increase IR induced DNA double-strand breaks, making it appear inert to irradiated non-HER2 expressing cells. At these dose levels, trastuzumab did not result in any significant radiosensitization. Moreover, doses as low as 2 nM T-DM1 radiosensitized OE19 cells (HER2+) as they accumulated in the G₂/M phase of the cell cycle (FIG. 3d,f).

ADC More Effectively Radiosensitize Tumours than Free Drugs.

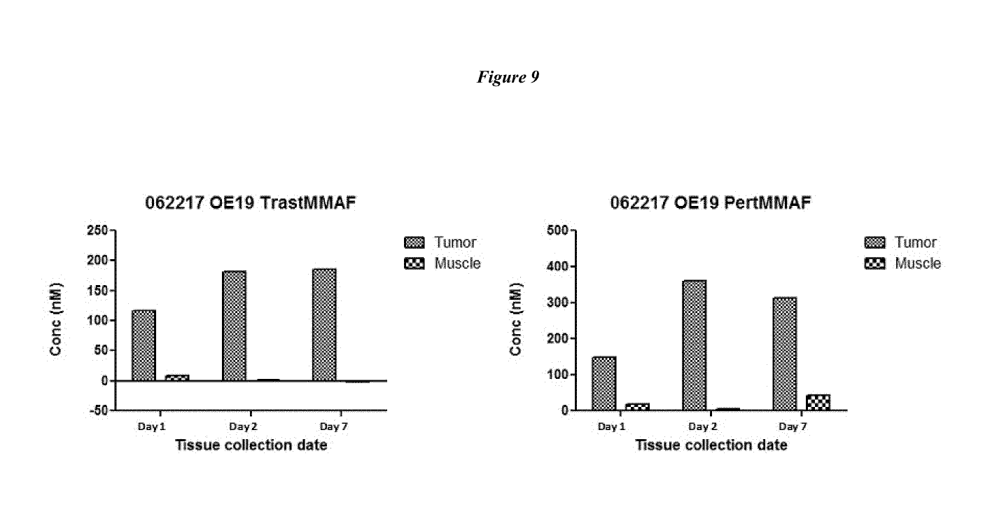

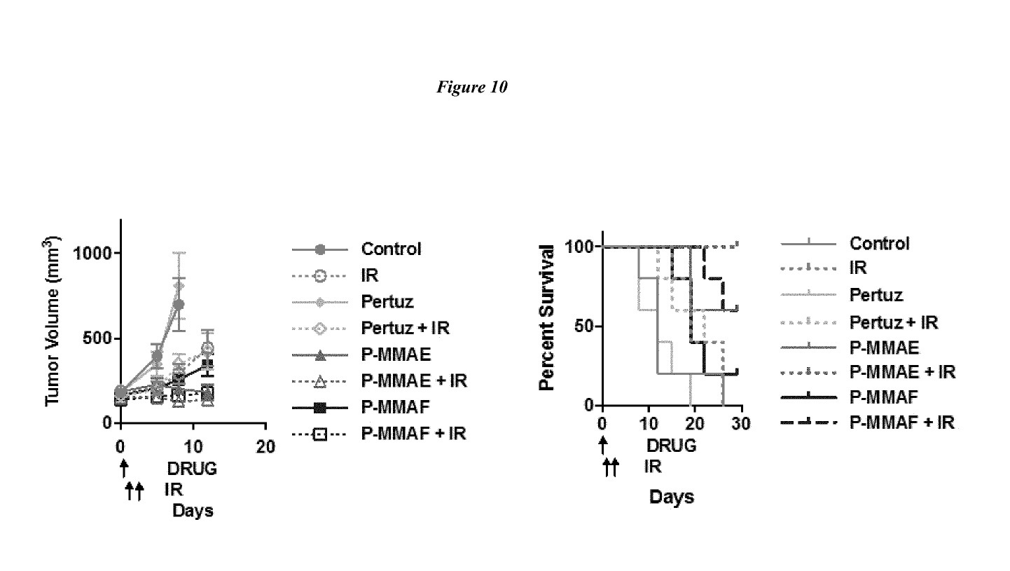

We then tested the efficacy of combining ErbB targeted delivery of anti-tubulins with IR in tumour xenografts. We first focused on our MMAE synthesized cetuximab and trastuzumab ADC since Cy5 labelling allows for in vivo ADC visualization, tracking and serves as a surrogate for tumour drug delivery following intravenous injection in mice. Mice bearing OE19 tumours were intravenously injected with Cy5-labelled C-MMAE or T-MMAE (FIG. 4a). In these HER2 tumours, T-MMAE demonstrated greater Cy5 signal accumulation within tumours compared with C-MMAE. MMAE target ‘hit’ was validated by staining tumours for the G₂/M marker, pS10 of Histone H3. T-MMAE resulted in increased pS10 of Histone H3 in tumours while cetuximab conjugation blocked MMAE anti-tubulin activity. Sectioned tumour xenografts demonstrated T-MMAE Cy5 fluorescence localized to areas of tumour cells and not stroma, an important pre-requisite for radiosensitizers to improve the therapeutic ratio. T-MMAE pharmacokinetics, measured by loss of Cy5 fluorescence in the blood was comparable to that of similar Cy5-labelled antibody alone (FIG. 4b)³⁶. Conversely in EGFR expressing tumours HNC (CAL-27, SCC-61, SCC-35 and SQ-9G), NSCLC (A549) and colorectal (HCT-116), tumour xenografts showed Cy5 signal accumulation for up to 72 h after C-MMAE injection while tumours from low EGFR LN-229 did not (FIG. 4c, Supplementary FIG. 9). Irradiation of tumour xenografts did not appreciably influence C-MMAE accumulation in tumours. EGFR+tumour xenografts from mice treated with C-MMAE showed increased accumulation in G₂/M by pS10 Histone H3 staining compared with cetuximab, verifying delivery of active MMAE to tumours (FIG. 4d). Importantly, tumour MMAE concentrations from CAL-27 xenografts injected with C-MMAE or T-MMAE confirmed restriction of released drug to EGFR+cells by cetuximab conjugation as opposed to trastuzumab conjugation (FIG. 4e). Next, we assessed the efficacy of anti-ErbB antibody MMAE conjugates in combination with IR on tumour regression. Mice with CAL-27 tumours were treated with vehide, free MMAE, cetuximab or C-MMAE on day 0 (FIG. 40. Cetuximab and C-MMAE were injected at a dose of 0.5 nmol˜3.6 and ˜4 mg kg⁻¹, respectively). Since an average of four molecules of MMAE were conjugated to each antibody molecule, a dose of 2 nmol of free MMAE was injected to maintain MMAE dose equivalence to C-MMAE. Because C-MMAE persisted in murine xenografts for up to 72 h (FIG. 4c), 3 Gy was given on days 1 and 2. In non-irradiated tumours, both free MMAE and cetuximab alone delayed tumour growth, which was further slowed by C-MMAE. Importantly, delivering IR with C-MMAE significantly increased tumour growth delay, P<0.0001 compared with all other experimental groups by day 35 post initiation of treatment (Supplementary Table 2). To specifically ascertain the advantage of using ADC in combination with IR, we tested C-MMAE compared with co-administered free MMAE and cetuximab (FIG. 4g). Interestingly in irradiated groups, mice receiving C-MMAE had significantly smaller tumours than those receiving co-administered cetuximab and free MMAE, P<0.05 by day 28 (Supplementary Table 3). In both experiments, mice tolerated therapies well as measured by weekly weights (Supplementary FIG. 10). These results reveal the advantages of using antibody conjugated MMAE as a radiosensitizer as opposed to the combination of antibodies with cytotoxic drugs on tumour control and is line with recent negative results from phase III clinical trials^{37,38}.

T-DM1 and RI Prolong HER2+but not HER2− Tumour Control.

Given these findings with C-MMAE and T-MMAE in EGFR and HER2+tumour xenografts, we next evaluated T-DM1 since it is already clinically approved and its safety established in women with metastatic breast cancer′. First, we compared the potency of auristatins and maytansinoids conjugated to trastuzumab. In HER2+OE19 and CALU3 cells, T-MMAE and T-DM1 had similar potency (FIG. 5a, Supplementary FIG. 11). In OE19 HER2+tumour xenografts, a single dose of 1 nmol of either T-MMAE or T-DM1 ˜7.7 and ˜7.4 mg kg⁻¹, respectively) were equally efficacious in slowing tumour growth compared with control or trastuzumab by day 14 (FIG. 5b, Supplementary Table 4). While T-DM1 increased apoptosis, it also increased the number of cells in radiosensitive G₂/M phase of the cell cycle, as measured by pS10 Histone H3 (FIG. 5c). Given the equivalence of T-MMAE and T-DM1 in cell culture and tumour xenografts, we then focused on optimizing dosing of T-DM1 and IR to determine if combining the two treatments would produce long term tumour control specifically in HER2+tumour xenografts. We dose reduced T-DM1 to 0.25 nmol (−1.9 mg kg⁻¹) delivered once (day 0). On the basis of our above results with our Cy5-labelled ADC, ADC localized within tumours for up to 72 h. Therefore, we delivered 2.5 Gy of IR on days 1, 2 and 3 after T-DM1 injection. In both OE19 and NCI N87 HER2+tumour xenografts, this regimen resulted in significantly prolonged tumour regression (FIG. 5d), increased. By day 22 in OE19 and day 32 in NCI N87 tumour xenografts, doubling time (Table 1, Supplementary Table 5), T-DM1 combined with IR was superior to all other treatment while mice maintained their weight (Supplementary FIG. 10). regimens (Supplementary Tables 6 and 7). In stark contrast, HER2− HCT116 tumour xenografts showed no benefit of adding T-DM1 to IR (FIG. 5d, Table 1, Supplementary Table 8). Of potential clinical value, long term tumour control was observed when T-DM1 was combined with IR in HER2+ tumours (FIG. 5e, Supplementary Table 9).

### Discussion

Unresectable, locally advanced cancers continue to pose a therapeutic challenge¹⁻³. The most effective chemoradiotherapy strategies to date integrate non-targeted cytotoxic chemotherapies^{3-9,39,40}. However, such therapeutic intensification increases normal tissue toxicities often precluding further radiotherapy or chemotherapy dose escalation¹⁰. To mitigate treatment related side effects and allow for more potent radiosensitizing chemotherapies, tumour targeted radio-sensitization approaches are required. While targeted drug therapies have advanced for certain cancer patients with specific mutations, outside of cetuximab none have demonstrated unequivocal clinical utility with radiotherapy when compared directly with non-targeted cytotoxic chemotherapies^{12,13,41,42}. Frustratingly, adding cetuximab to cytotoxic chemo-radiotherapy in NSCLC or HNC patients failed to improve outcomes^{37,38}.

TABLE 1
T-DM1 combined with IR increases HER2+ tumor control.

| | OE19 | NCI N87 | HCT116 |
|---|---|---|---|
| Control | 7 (5-14) | 9 (7-16) | 3 (2-5) |
| Trastuz | 6 (4-8) | 14 (10-21) | 4 (2-8) |
| T-DM1 | 11 (8-17) | 23 (21-25) | 3 (2-5) |
| IR | 9 (7-11) | 14 (11-20) | 6 (4-9) |
| Trastuz + IR | 14 (10-24) | 19 (16-22) | 7 (5-12) |
| T-DM1 + IR | 66 (33+) | 113 (82-179) | 7 (5-12) |

Tumour doubling time in days (mean, 95% CI) of mice in FIG. 4d bearing OE19 or NCI N87 (HER2+) or HCT116 (HER2−) tumour xenografts.

While inhibiting ErbB signalling is an appealing approach to radiosensitize tumours, attempted blockage of receptor tyrosine kinase signalling results in activation of bypass pathways, that is the ‘whack a mole’ problem^{21,24,25}. To overcome this, we propose an alternative ErbB mediated radiosensitization paradigm based on ErbB directed ADC that is more potent and potentially superior to signal inhibition. We initially evaluated the auristatin class (MMAE) of anti-tubulins as ADC-based radiosensitizers for three reasons. First, it is the ‘warhead’ of brentuximab vedotin, a clinically used ADC for CD3O lymphomas that is attached through a cathepsin B sensitive and self-immolative linker. Moreover, this allowed for direct measurement of tumour drug delivery (FIG. 4)³⁵. Second, we have previously demonstrated that in addition to its cytotoxic activity, MMAE is a potent radiosensitizer³³. Finally, Cy5 labelling during ADC synthesis allowed for non-invasive imaging of ADC localization and served as a marker for antibody targeting which was validated by tumour drug delivery and activity of blocking cells in G₂/M. Our synthesized ADC showed tumour accumulation of T-MMAE or C-MMAE in a receptor-restricted manner which resulted in tumour selective MMAE radiosensitization. Given our findings with C-MMAE and T-MMAE, we extended our studies to the clinically approved ADC, T-DM1. As with auristatins, we found that the maytansinoid class of anti-tubulins also were potent radiosensitizers as either free drug or as the trastuzumab ADC, T-DM1 (FIGS. 3 and 5).

Delivering ADC with IR has several advantages that can result in improved patient outcomes: (1) Combinatorial therapy attacks cancer cells by multiple mechanisms decreasing the risk of tumour resistance emerging. (2) Instead of higher individual doses of a single agent to achieve effective tumour kill, combinatorial therapy allows for dose reduction of each individual modality thereby decreasing the toxicities inherently associated with each therapy. (3) concurrent delivery of full dose chemotherapy and radiotherapy allow for attacking not only known local disease but also potential micrometastases. (4) Ideally, chemotherapies used in conjunction with radiotherapy will be highly potent alone, synergize with IR, and be spatially targeted to tumours and not normal tissue. (5) The precise timing with which IR can be delivered is valuable for defining the temporal window(s) when radiosensitization should be maximal. Auristatin and maytansinoid based ADC fulfill these criteria. Currently, there are only two indications for which ADC has shown clinical value, CD30 lymphomas and HER2 metastatic breast cancer^{30,31}. Our studies and in particular those involving T-DM1, suggest integrating ADC in the curative setting for locally advanced solid tumours. HER2 overexpression occurs in a proportion of lung, esophageal, gastric and bladder cancers, which are treated with concurrent chemo-radiotherapy^{23,43,44}. On the basis of our findings, T-DM1 provides potent and tumour selective radiosensitization that warrants speedy clinical evaluation and may help expedite the evolution of tumour radiosensitization from decades old non-targeted cytotoxins to a biomarker-driven tumour-targeted paradigm.

### Methods

Cells and Reagents.

All cell lines used in these studies are summarized in Supplementary Table 1. Human HNC (CAL-27, SCC-25), NSCLC (A549, CALU3), colorectal (HCT-116), gastric (NCI N87), glioma (LN229) and breast (BT474) cancer cell lines were obtained from American Type Culture Collection. Human esophageal cancer line OE19 was obtained from Sigma-Aldrich. Human HNC SCC-35, SCC61 and SQ-9G was kindly provided from Ralph Weichselbaum, University of Chicago. CAL-27, A549 and HCT116, cells were cultured in DMEM supplemented with 10% FBS. SCC-61 was cultured in DMEM/F12 supplemented with 20% FBS and 400 ng nil −1 hydrocortisone. OE19 and NCI N87 cells were cultured in RPMI supplemented with 10% FBS. CALU3 cells were cultured in MEM supplemented with 10% FBS. On receipt, each cell line was expanded, cryopreserved as low passage stocks and routinely tested for mycoplasma. Cisplatin (Enzo Biosciences), paclitaxel (Sigma), MMAE (Concortis) and mertansine (Abcam) were reconstituted in DMSO. Clinical-grade erlotinib, lapatinib, cetuximab, trastuzumab, ado-trastuzumab emtansine (T-DM1) were obtained from UCSD Moores Cancer Center pharmacy.

Synthesis of MMAE and Cy5 Anti-ErbB Antibodies Conjugates.

A solution (1 ml, 2 mg ml⁻¹) of cetuximab (Erbitux, ImClone) or trastuzumab (Herceptin, Roche) was treated with sodium bicine buffer (100 μl, 1 M pH 8.3) and sodium diethylenetriaminepentaacetic acid (10 100 μl, 100 mM pH 7). Following reduction with four equivalents of tris(carboxyethyl)phosphine (TCEP) at 37° C. for 2 h, the solution was added to four equivalents of maleimidocaproyl-valine-citrulline-PABA-MMAE (MC-VC-MMAE)33. After 30 min at room temperature, Cy5-maleimide (2 equivalents) was added and after a further 30 min, gel-filtered (Sephadex G25, 0.6 g) eluting with PBS. Following centrifugal concentration (Centricon 30 kDa MWCO) to 500 μl, the concentrations of antibody and Cy5 were determined by absorbance using extinction coefficients of 210,000 M⁻1 cm⁻1 (cetuximab) or 225,000 M⁻¹ cm⁻¹ (trastuzumab) at 280 nm and 12,500 M⁻¹ cm⁻¹ and 250,000 M⁻¹ cm⁻¹ at 280 and 650 nm, respectively, for Cy5. Hydrophobic interaction chromatography (HIC) of a reaction sample after labelling with MMAE revealed nine peaks corresponding to antibody modified with 0-8 MMAE derivatives if up to four disulfides are reduced by TCEP per antibody³⁴ (Supplementary FIG. 2a,c). Subsequent Cy5-maleimide labelling gave 650 nm absorbance to each peak apart from that labelled with eight MMAE as no cysteines are available for further conjugation (Supplementary FIG. 2b,d). Drug loading was measured by denaturing reverse-phase HPLC of the reaction mix before addition of Cy5 maleimide, following reduction of any remaining intersubunit disulfides with 50/TIM DTT for 30 min (ref. 45). Peaks corresponding to light or heavy chains^{46,42} with 0-3 MMAE were identified by electro-spray mass spectroscopy (Supplementary FIG. 2e-h) and peak areas at 280 nm were integrated and weighted to calculate the drug loading²⁶. Modified light chain (L1) and unmodified H chain (HO) were not resolved for trastuzumab so MMAE loading is an underestimate. No free MC-VC-MMAE was detected by HPLC following gel filtration. This conjugation chemistry that we utilized has subsequently been shown to undergo a slow retro-Michael reaction resulting in loss of the MMAE-linker from the antibody in the circulation and potential off-target toxicity. Modifications have been devised to decrease premature release of the warhead but have yet to be clinically approved, so we retained the established linker′.

Cy5 Fluorescence Imaging.

Cells were exposed to Cy5-labelled cetuximab-MMAE or trastuzumab-MMAE for 30 min in media with 1% serum. Cells were then washed with PBS and incubated in media with 10% serum. At indicated times, cells were fixed in 4% paraformaldehyde and then stained with DAPI. Cells were imaged using a Nikon MR confocal microscope.

ADC Cell Binding.

Cells were collected and resuspended in cold PBS with 5% BSA. Cy5-labelled cetuximab-MMAE or trastuzumab-MMAE was added to the cells at indicated concentrations for 15 min on ice. Cells were washed, resuspended in PBS with 5% BSA and 0.5 ml propidium iodide, and analysed by flow cytometry.

Cell Cycle.

Cells were treated with MMAE, mertansine, cetuximab or cetuximab-MMAE, trastuzumab, trastuzumab-MMAE or T-DM1 for 24 h and then fixed in methanol. Cells were treated with RNAse, stained with propidium iodide (PI) and analysed by FACS using FloJo software.

Alamar Blue Assay.

Cells were plated in 96-well plates and exposed to a range of concentrations of MMAE, mertansine, cisplatin or paclitaxel, cetuximab, cetuximab-MMAE, trastuzumab-MAME or T-DM1 for 72 h. Alamar Blue (resazurin) was added to the cells and allowed to incubate for 2-4 h at 37° C. Plates were analysed using a plate reader with fluorescence measured at 560 nm.

Clonogenic Assay.

Cells were treated with MMAE, mertansine, cetuximab or cetuximab-MMAE for 24 h and then irradiated with 2 Gy. Following IR, cells were counted, re-plated at varying cell numbers in drug-free media. 10-14 days after initial seeding formed colonies were methanol fixed, stained with crystal violet and counted. Surviving fraction at 2 Gy (SF2) was calculated as the fraction of cells surviving 2 Gy compared with non-irradiated cells.

Neutral Comet Assay.

Cells were treated with indicated times doses of MMAE, mertansine, cetuximab, cetuximab-MMAE, trastuzumab, trastuzumab-MMAE or T-DM1 overnight, and then irradiated with 6 Gy. Cells were collected 15 min post IR, suspended in agarose gel and lysed per assay directions (Trevigen). Samples underwent electrophoresis under neutral conditions and were then stained with Sybr Green. Comet tails were counted in multiple fields (>60 cells per sample) and analysed using CometScore (TriTek Corp). Comet tail length was normalized to vehicle-treated, non-irradiated cells.

Immunoblotting.

Cells were collected and lysed in RIPA buffer (20 niM Tris pH 8, 150 mM NaCl, 5 niM EDTA, 1% Triton X-100) with protease and phosphatase inhibitors (Complete Protease Inhibitor Cocktail and Phos-Stop, Roche). Lysate protein was quantitated by BCA technique (Pierce). Twenty micrograms of lysate underwent electrophoresis using 4-12% Bis-Tris gels (Life Technologies), transferred to PVDF membranes (iBlot) and incubated with indicated primary antibodies HER2, GAPDH (Cell Signaling Technology, catalogue numbers 2242 and 2118) and EGFR (Millapore, catalogue number 06-847) at dilutions of 1:1,000, 1:3,000 and 1:3,000, respectively. Blots were developed by ECL (Pierce). Uncropped blots are shown in relevant Supplementary Figures.

Immunohistochemistry.

Tissue sections were cut from blocks of formalin-fixed paraffin embedded xenografts. Four micron thick tissue sections were stained with antibodies to phosphohistone H3 (pH3, Abcam, catalogue number ab32107) and cleaved caspase-3 (C1C3, Cell Signaling Technology, catalogue number 9661) and used at dilutions of 1:300 and 1:900, respectively. Slides were stained on a Ventana Discovery Ultra (Ventana Medical Systems, Tucson, Ariz., USA). Antigen retrieval was performed using CC1 for 24-40 min at 95° C. The primary antibodies were incubated on the sections for 1 h at 37° C. Primary antibodies were visualized used DAB as a chromagen using the UltraMap system (Ventana Medical systems) followed by hematoxylin as a counterstain. Slides were rinsed, dehydrated through alcohol and xylene and coverslipped.

Whole Slide Scanning and Immunostaining Quantitation.

Immununostained slides were scanned using an Axio Scan.Z1 (Zeiss; Oberkochen, Germany). Axio Scan uses the software Zen2 (Zeiss) for automatic thresholding and tissue detection. The entire tissue section encompassing the xenograft was scanned at 40×(0.95 numerical aperture) using the default stitching parameters to combine the individual tiles into a single image. Whole-slide images were imported into Definiens Software for quantitative analysis (Definiens; Munich, Germany). Using Definiens Tissue, tumour regions of interest (ROI) were chosen per slide based on the pH3 immunostain. The software uses the color contrast of the DAB and hematoxylin counterstain to determine the basic ROI. Training was done to exclude non-tumour tissue including necrotic regions and mouse stroma. The ROI defined for the pH3 stain was also used to analyse the adjacent section stained for C1C3. For analysis of pH3 (a nuclear stain), the total number of nuclei and the total number of nuclei showing DAB staining (pH3 positive cells) were recorded within each ROI. The percent of positive nuclei was then calculated for each sample. For analysis of C1C3 (a cytoplasmic stain), mean DAB chromagen intensity was measured and calculated within the ROI for each sample.

In Vivo Tumour Xenograft Optical Imaging.

All animal work was done in compliance with the University of California San Diego Institutional Animal Use and Care Committee. Six-to-eight-week-old female athymic nu/nu mice purchased from the University of California San Diego Animal Care Program breeding colony were injected subcutaneously into the bilateral upper thighs with 5×106 CAL-27, SQ9-G, SCC-35, SCC-61, A549, HCT-116, OE19 or LN229 tumour cells in a 1:1 Matrigel (BD) and PBS solution. After tumours grew to >100 mm3 they were injected with 0.5 nmol of Cy-labelled cetuximab-MMAE, trastuzumab-MMAE (.'4 and 3.9 mg kg⁻¹, respectively) as described above. For imaging, mice were anaesthetized (1:1 mixture of 100 mg ml—1 of ketamine and 5 mg ml—1 of midazolam). Animals were imaged using a Maestro Small Animal Imager (CRI) with excitation filter of 620/22 and 645 nm long-pass emission filter with dichroic filter tuned to 670 nm. Imaging was done both with skin on and after skin removal to decrease autofluorescence and scattering. For blood clearance studies, athymic nu/nu mice were anaesthetized with isoflurane and dosed with 5 nmol of the antibody conjugates (.'36.4 trastuzumab and 38.6 mg kg⁻¹ T-MMAE). At various time points after injection, the tail was pricked and a small volume (5-10 μl) of blood was collected in a heparinized hematocrit tube. Fluorescent images were taken using the Maestro, with the filters mentioned above, and the integrated fluorescent intensity was measured using Image J.

In Vivo Tumour Xenograft Experiments.

CAL-27, OE19, NCI N87 and HCT116 tumour xenografts were established and tumour growth was measured with digital calipers. Tumour volume was measured blindly to treatment group and calculated using the formula as 1/2×length×width. Mice were randomized into groups once the average tumour volume reached >100=113. Mice were assigned to indicated groups in ‘Results’. MMAE, cetuximab, trastuzumab, C-MMAE, T-MMAE or T-DM1 was intravenously injected in 50 μl. For irradiated mice, the tumour-bearing hindlimbs were focally irradiated while the remainder of the mouse was shielded from IR with custom designed lead blocking >95% of the dose as verified by dosimeters placed on the mouse. Free MMAE was injected on an equimolar basis to C-MMAE in final volume of 3% DMSO. Drug doses and IR fractionation are as indicated in ‘Results’. To prevent unnecessary morbidity, mice were killed if tumour length exceeded 15 mm per protocol Nonlinear regression least squares fit was used to calculate tumour volume doubling times. Tumour doubling times were extrapolated for T-DM1+IR groups where tumour doubling was been achieved. To minimize the number of mice used, tumours were grown in the bilateral flanks. For CAL-27 tumour xenografts in experiment FIG. 4f, the number of mice and tumours per group were: control (5 mice, 10 tumours), IR (5 mice, 10 tumours), MMAE (4 mice, 8 tumours) MMAE+IR (5 mice, 10 tumours), cetuximab (5 mice, 10 tumours), cetuximab+IR (5 mice, 10 tumours), C-MMAE (4 mice, 8 tumours), C-MMAE+IR (5 mice, 10 tumours). For CAL-27 tumour xenografts in experiment FIG. 4g, the number of mice and tumours per group were: control (4 mice, 8 tumours), IR (5 mice, 10 tumours), cetuximab+MMAE (5 mice, 10 tumours) cetuximab+MMAE+IR (5 mice, 10 tumours), C-MMAE (5 mice, 10 tumours), C-MMAE+IR (5 mice, 10 tumours). For OE19 tumour xenografts in experiment FIG. 5b, the number of mice and tumours per group were: control (5 mice, 10 tumours), trastuzumab (5 mice, 10 tumours) T-MMAE (5 mice, 10 tumours), T-DM1 (5 mice, 10 tumours). For OE19 tumour xenografts in experiment FIG. 5d,e and Table 1, the number of mice and tumours per group were: control (5 mice, 10 tumours), IR (5 mice, 10 tumours), trastuzumab (5 mice, 10 tumours), trastuzumab+IR (5 mice, 10 tumours), T-DM1 (5 mice, 10 tumours) and T-DM1+IR (5 mice, 10 tumours). For NCI N87 tumour xenografts in experiment FIG. 5d,e and Table 1, the number of mice and tumours per group were: control (2 mice, 4 tumours), IR (3 mice, 6 tumours), trastuzumab (3 mice, 6 tumours), trastuzumab+IR (4 mice, 8 tumours), T-DM1 3 mice, 6 tumours) and T-DM1+IR (4 mice, 8 tumours). For HCT116 tumour xenografts in experiment FIG. 5d,e and Table 1, the number of mice and tumours per group were: control (3 mice, 6 tumours), IR (4 mice, 8 tumours), trastuzumab (3 mice, 6 tumours), trastuzumab+IR (3 mice, 6 tumours), T-DM1 (3 mice, 6 tumours) and T-DM1+IR (4 mice, 8 tumours).

Tumour Xenograft Drug Measurement.

Tumours were excised, weighed and homogenized in 10 volumes of PBS with a point sonicator (Fisher Scientific) using an amplitude range of 5-15% for a maximum of 20 s while on ice. The homogenates were centrifuged (14 g, 10 min), then the supernatants were collected and diluted two-fold by addition of 2% acetic acid in acetonitrile, then centrifuged again (14 g, 10 min). MMAE concentration was determined by LC-MS/MS with Luna-2 C18 column and Agilent Trap XCT mass spectrometer and extracted fragment ion currents at 686.4 and 506.4 were integrated and combined to improve sensitivity.

Statistical Analysis.

Unpaired two-sided t-tests were performed for IC₅₀ and radiosensitization experiments in cell culture. In tumour regression studies, two-way ANOVA analysis was performed with Tukey's multiple comparison group. Survival curves were analysed log-rank. Tumour doubling volume times were analysed by determining 95% confidence intervals. All statistical analyses were performed using Prism software (GraphPad). Statistical analysis for all tumour xenograft data are presented in Supplementary Tables 2-9.

### REFERENCES

1. Marur, S. & Forastiere, A. A. Challenges of integrating chemotherapy and targeted therapy with radiation in locally advanced head and neck squamous cell cancer. Curr. Opin. Oncol. 222, 206-211 (2010).
2. Laine, A. M., Westover, K D. & Choy, H. Radiation therapy as a backbone of treatment of locally advanced non-small cell lung cancer. Semin. Oncol. 41, 57-68 (2014).
3. Cohen, D. J. & Leichman, L. Controversies in the treatment of local and locally advanced gastric and esophageal cancers. J. Clin. Oncol. 33, 1754-1759 (2015).
4. Herskovic, A. et al. Combined chemotherapy and radiotherapy compared with radiotherapy alone in patients with cancer of the esophagus. N. Engl. J. Med. 326, 1593-1598 (1992).
5. Epidermoid anal cancer: results from the UKCCCR randomised trial of radiotherapy alone versus radiotherapy, 5-fluorouracil, and mitomycin. UKCCCR Anal Cancer Trial Working Party. UK Co-ordinating Committee on Cancer Research. Lancet 348, 1049-1054 (1996).
6. Al-Sarraf, M. et al. Chemoradiotherapy versus radiotherapy in patients with advanced nasopharyngeal cancer: phase III randomized Intergroup study 0099. J. Clin. Oncol. 16, 1310-1317 (1998).
7. Morris, M. et al. Pelvic radiation with concurrent chemotherapy compared with pelvic and para-aortic radiation for high-risk cervical cancer. N. Engl. I Med. 340, 1137-1143 (1999).
8. Furuse, K et al. Phase III study of concurrent versus sequential thoracic radiotherapy in combination with mitomycin, vindesine, and cisplatin in unresectable stage III non-small-cell lung cancer. J. Clin. Oncol. 17, 2692-2699 (1999).
9. Macdonald, J. S. et al. Chemoradiotherapy after surgery compared with surgery alone for adenocarcinoma of the stomach or gastroesophageal junction. N. Engl. J. Med. 345, 725-730 (2001).
10. Moding, E. J., Kastan, M. B. & Kirsch, D. G. Strategies for optimizing the response of cancer and normal tissues to radiation. Nat. Rev. Drug Discov. 12, 526-542 (2013).
11. Kesari, S. et al. DNA damage response and repair: insights into strategies for radiation sensitization of gliomas. Future Oncol. 7, 1335-1346 (2011).
12. Liauw, S. L., Connell, P. P. & Weichselbaum, R. R. New paradigms and future challenges in radiation oncology: an update of biological targets and technology. Sci. Transl. Med. 5, 173sr2 (2013).
13. Morris, Z. S. & Harari, P. M. Interaction of radiation therapy with molecular targeted agents. J. Clin. Oncol. 32, 2886-2893 (2014).
14. Dutta, P. R. & Many, A. Cellular responses to EGFR inhibitors and their relevance to cancer therapy. Cancer Lett. 254, 165-177 (2007).
15. Bonner, J. A. et al. Radiotherapy plus cetuximab for locoregionally advanced head and neck cancer: 5-year survival data from a phase 3 randomised trial, and relation between cetuximab-induced rash and survival. Lancet Oncol. 11, 21-28 (2010).
16. Selzer, E. & Kornek, G. Targeted drugs in combination with radiotherapy for the treatment of solid tumors: current state and future developments. Expert Rev. Clin. Pharmacol. 6, 663-676 (2013).
17. Le, Q. T. & Raben, D. Integrating biologically targeted therapy in head and neck squamous cell carcinomas. Semin. Radiat. Oncol. 19, 1953-1962 (2009).
18. No, M., Choi, E. J. & Kim, I. A. Targeting HER2 signaling pathway for radiosensitization: alternative strategy for therapeutic resistance. Cancer Biol. Ther. 8, 2351-2361 (2009).
19. Thou, H. et al. Effects of the EGFR/HER2 kinase inhibitor GW572016 on EGFR- and HER2-overexpressing breast cancer cell line proliferation, radiosensitization, and resistance. Int. J. Radiat. Oncol. Biol. Phys. 58, 344-352 (2004).
20. Liang, K et al. Sensitization of breast cancer cells to radiation by trastuzumab. Mol. Cancer Ther. 2, 1113-1120 (2003).
21. Wheeler, D. L., Dunn, E. F. & Harari, P. M. Understanding resistance to EGFR inhibitors-impact on future treatment strategies. Nat. Rev. Clin. Oncol. 7, 493-507 (2010).
22. Brand, T. M. et al. AXL mediates resistance to cetuximab therapy. Cancer Res. 74, 5152-5164 (2014).
23. Saki, M., Toulany, M. & Rodemann, H. P. Acquired resistance to cetuximab is associated with the overexpression of Ras family members and the loss of radio-sensitization in head and neck cancer cells. Radiother. Oncol. 108, 473-478 (2013).
24. Lavaud, P. & Andre, F. Strategies to overcome trastuzumab resistance in HER2-overexpressing breast cancers: focus on new data from clinical trials. BMC Med. 12, 132 (2014).
25. Creedon, H. et al. Exploring mechanisms of acquired resistance to HER2 (human epidermal growth factor receptor 2)-targeted therapies in breast cancer. Biochem. Soc. Trans. 42, 822-830 (2014).
26. Doronina, S. 0. et al. Development of potent monoclonal antibody auristatin conjugates for cancer therapy. Nat. Biotechnol. 21, 778-784 (2003).
27. MuBard, A. Maturing antibody-drug conjugate pipeline hits 30. Nat. Rev. Drug. Discov. 12, 329-332 (2013).
28. Sievers, E. L & Senter, P. D. Antibody-drug conjugates in cancer therapy. Annu. Rev. Med. 64, 15-29 (2013).
29. Girish, S. et al. Clinical pharmacology of trastuzumab emtansine(T-DM1): an antibody-drug conjugate in development for the treatment of HER2-positive cancer. Cancer Chemother. Pharmacol. 69, 1229-1240 (2012).
30. Verna, S. et al. Trastuzumab emtansine for HER2-positive advanced breast cancer. N. Engl. J. Med. 367, 1783-1791 (2013).
31. Chen, X., Soma, L A. & Fromm, J. R. Targeted therapy for Hodgkin lymphoma and systemic anaplastic large cell lymphoma-. focus on brentuximab vedotin. Onco. Targets Ther. 7, 45-56 (2013).
32. Okeley, N. M., Alley, S. C. & Senter, P. D. Advancing antibody drug conjugation: from the laboratory to a clinically approved anticancer drug. Hematol. Oncol. Clin. North Am. 28, 13-25 (2014).
33. Buckel, L et al. Tumor radiosensitization by monomethyl auristatin E: mechanism of action and targeted delivery. Cancer Res. 75, 1376-1387 (2015).
34. Hamblen, K. J. et al. Effects of drug loading on the antitumor activity of a monoclonal antibody drug conjugate. Clin. Cancer Res. 10, 7063-7070 (2004).
35. Lyon, R. P., Meyer, D. L., Setter, J. R. & Senter, P. D. Chapter six-conjugation of anticancer drugs through endogenous monoclonal antibody cysteine residues. Methods Enzymol. 502, 123-138 (2012).
36. Sanderson, R. J. et al. In vivo drug-linker stability of an anti-CD30 dipeptide-linked auristatin immunoconjugate. Clin Cancer Res. 11, 843-852 (2005).
37. Ang, K. K. et al. Randomized phase III trial of concurrent accelerated radiation plus cisplatin with or without cetuximab for stage III to IV head and neck carcinoma: RTOG 0522. J. Clin. Oncol. 20, 2940-2950 (2014).
38. Bradley, J. D. et al. Standard-dose versus high-dose conformal radiotherapy with concurrent and consolidation carboplatin plus paclitaxel with or without cetuximab for patients with stage IIIA or IIIB non-small-cell lung cancer (RTOG 0617): a randomised, two-by-two factorial phase 3 study. Lancet Oncol. 16, 187-199 (2015).
39. Ley, J. et al. Cisplatin versus cetuximab given concurrently with definitive radiation therapy for locally advanced head and neck squamous cell carcinoma. Oncology 85, 290-296 (2013).
40. Koutcher, L et al. Concurrent cisplatin and radiation versus cetuximab and radiation for locally advanced head-and-neck cancer. Int. J. Radiat. Oncol. Phys. 81, 915-922 (2011).
41. Lin, S. H. et al. Opportunities and challenges in the era of molecularly targeted agents and radiation therapy. J. Natl. Cancer Inst. 105, 686-693 (2013).
42. Liu, F. F. et al. Lessons learned from radiation oncology trials. Clin. Cancer Res. 19, 6089-6100 (2013).
43. Swanton, C., Futreal, A. & Eisen, T. HER2-targeted therapies in non-small cell lung cancer. Clin. Cancer Res. 12, 4377s-4383s (2006).
44. Okines, A., Cunningham, D. & Chau, I. Targeting the human EGFR family iesophagogastric cancer. Nat. Rev. Clin. Oncol. 8, 492-503 (2011).
45. Sun, M. M. C. et al. Reduction—alkylation strategies for the modification of specific monoclonal antibody disulfides. Bioconjug. Chem. 165, 1282-1290 (2005).
46. Ayoub, D. et al. Correct primary structure assessment and extensive glyco-profiling of cetuximab by a combination of intact, middle-up, middle-down and bottom-up ESI and MALDI mass spectrometry techniques. mAbs 5, 699-710 (2013).
47. Damen, C. W. N. et al. Electrospray ionization quadrupole ion-mobility time-of-flight mass spectrometry as a tool to distinguish the lot-to-lot heterogeneity in N-glycosylation profile of the therapeutic monoclonal antibody trastuzumab. J. Am. Soc. Mass Spectrom. 20, 2021-2033 (2009).
48. Lyon, R. P. et al. Self-hydrolyzing maleimides improve the stability and pharmacological properties of antibody-drug conjugates. Nat. Biotechnol. 32, 1059-1062 (2014).

The examples set forth above are provided to give those of ordinary skill in the art a complete disclosure and description of how to make and use the embodiments of the compositions, systems and methods of the disclosure, and are not intended to limit the scope of what the inventors regard as their disclosure. Modifications of the above-described modes for carrying out the disclosure that are obvious to persons of skill in the art are intended to be within the scope of the following claims. All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

1. A method for inducing radiosensitization comprising administering to a subject in need thereof a molecule comprising the formula:
A-T
wherein the molecule comprises:
an antibody A;
a radiosensitizing agent T, wherein said radiosensitizing agent is not an auristatin, including MMAE,
wherein said subject is radiosensitized.

2. The method of claim 1, wherein T is a maytansinoid.

3. The method of claim 1, wherein T is mertansine.

4. The method of claim 1, wherein said antibody is an anti-ErbB antibody.

5. The method of claim 4, wherein said antibody is selected from the group consisting of cetuximab, trastuzumab, and pertruzumab.

6. The method of claim 1, wherein said molecule comprises the formula:
A-Y-T
wherein the molecule comprises:
a cleavable linker Y.

7. The method of claim 6, wherein Y is selected from the group consisting of Val-Cit-(p-amido)benzyloxycarbonyl and maleimidocaproyl-valine-citrulline-paminobenzyloxycarbonyl (MC-VC-PABC).

8. The method of claim 1, wherein the molecule is administered prior to administration of a radiation therapy.

9. The method of claim 1, wherein the molecule is administered concurrently with administration of a radiation therapy.

10. The method of claim 6, wherein the cleavable linker is cleaved in vivo in the presence of a tumor in the subject.

11. A composition for inducing radiosensitization in a subject, wherein said composition comprises a molecule comprising the formula:
A-T
wherein the molecule comprises:
an antibody A;
a radiosensitizing agent T, wherein said radiosensitizing agent is not an auristatin, including MMAE.

12. The composition of claim 11, wherein T is a maytansinoid.

13. The composition of claim 11, wherein T is mertansine.

14. The composition of claim 11, where said antibody is an anti-ErbB antibody.

15. The composition of claim 14, wherein said antibody is selected from the group consisting of cetuximab, trastuzumab, and pertuzumab.

16. The composition of claim 11, wherein said molecule comprises the formula:
A-Y-T
wherein the molecule comprises:
a cleavable linker Y.

17. The composition of claim 16, wherein Y is selected from the group consisting of Val-Cit-(p-amido)benzyloxycarbonyl and maleimidocaproyl-valine-citrulline-paminobenzyloxycarbonyl (MC-VC-PABC).

18. The composition of claim 11, wherein said composition comprises a pharmaceutically acceptable excipient.

19. The composition of claim 11, wherein the molecule is administered prior to or concurrently with administration of a radiation therapy.

20. (canceled)

21. The composition of claim 16, wherein the cleavable linker is cleaved in vivo in the presence of a tumor in the subject.
